(19) United States
(12) Patent Application Publication
(10) Pub. No.: US 2013/0071357 A1
(43) Pub. Date: Mar. 21, 2013

(54) IDENTIFICATION, PROLIFERATION IN SITU, HARVESTING, SEPARATION, AND TRANSPLANTATION OF ADULT-DERIVED REGENERATIVE PLURIPOTENT TRANSITIONAL BLASTOMERE-LIKE STEM CELLS AND METHODS OF TREATMENT THEREOF

(71) Applicant: Henry E. Young, Macon, GA (US)

(72) Inventor: Henry E. Young, Macon, GA (US)

(73) Assignee: HEYGEN, LLC, Henderson, NV (US)

(21) Appl. No.: 13/365,880

(22) Filed: Feb. 3, 2012

Related U.S. Application Data
(63) Continuation-in-part of application No. 13/363,370, filed on Jan. 31, 2012, now abandoned.
(60) Provisional application No. 61/437,705, filed on Jan. 31, 2011.

(51) Int. Cl. C12N 5/078; A61K 35/14

(52) U.S. Cl. CPC C12N 5/0634; A61K 35/14
USPC 424/93.7; 435/366; 424/602

(57) ABSTRACT

Non-embryonic transitional blastomere-like stem cells are disclosed. Most preferably, such cells are obtained from the blood after induction by a plant-based compound to proliferate and reverse diapadese into the vasculature or from various tissues of postnatal mammals or humans (using tissue biopsied from the mammal or human), are in the range of 3-5 microns, have a normal karyotype, and do not spontaneously differentiate in situ (in vivo) or in serum-free medium without differentiation inhibitors. These non-embryonic transitional blastomere-like stem cells typically express CD66e, CEA-CAM-1, CD10, SSEA (SSEA-1, SSEA-3, and SSEA-4), telomerase, Sonic hedgehog, but do not typically express Nanog, Nanos, BCl-2 or CXCR-4. Such transitional blastomere-like pluripotent stem cells can be differentiated into epiblast-like stem cells, ectodermal, mesodermal, and endodermal tissues, but NOT placental tissues or germ cells. Moreover, when implanted into a mammal or human, such cells will not be teratogenic.

This application is a continuation-in-part of U.S. patent application Ser. No. 13/363,370 entitled “ADULT-DERIVED REGENERATIVE PLURIPOTENT TRANSITIONAL BLASTOMERE-LIKE STEM CELLS AND METHODS FOR IDENTIFICATION, PROLIFERATION, HARVESTING, SEPARATION, AND TRANSPLANTATION THEREOF,” filed 31 Jan. 2012. U.S. patent application Ser. No. 13/363,370 claims the benefit of and priority to U.S. Prov. Pat. App. Ser. No. 61/437,705, filed Jan. 31, 2011. Each of the above listed applications are incorporated herein by referent in their entireties.

### FIELD OF THE INVENTION

The field of the invention is stems cells and reagents for same, and especially as they relate to adult-derived transitional blastomere-like stem cells and regenerative pluripotent stem cells.

### BACKGROUND OF THE INVENTION

### Stem Cells

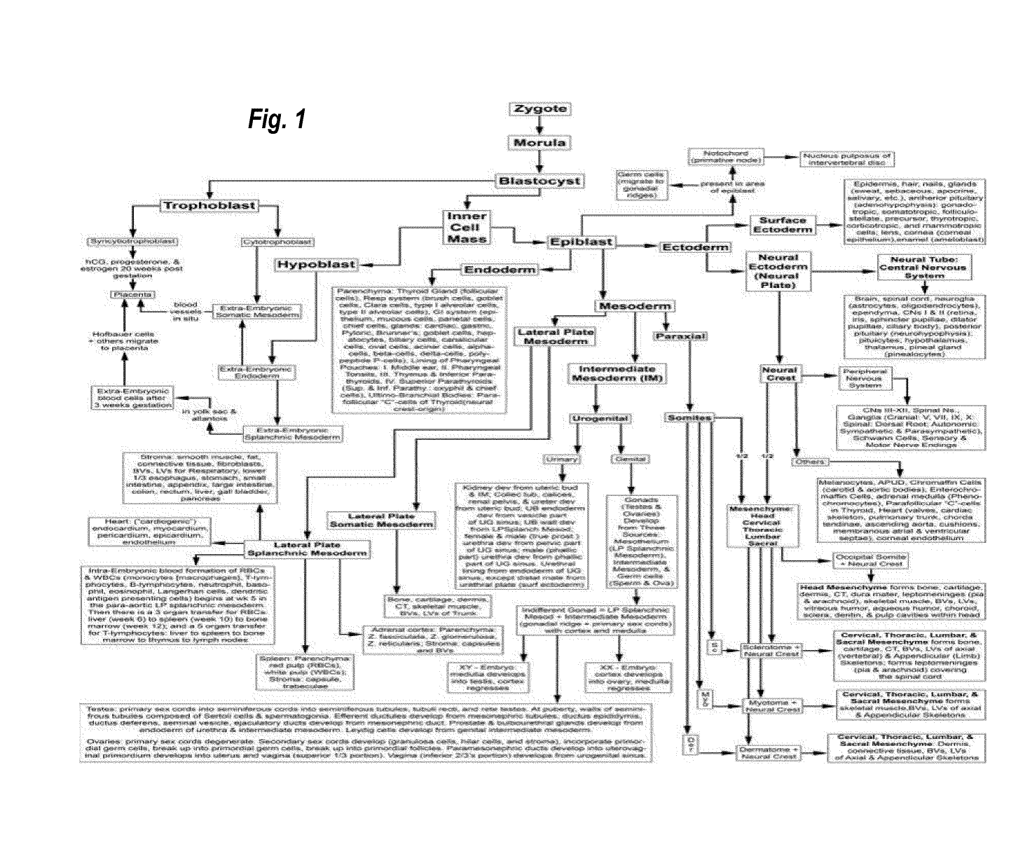

It is currently thought that mammalian cells progress from embryonic cell stages to fully developed cells through a sequence of totipotent blastomeric cells that transition into pluripotent epiblastic cells, which transition into germ layer lineage cells, which transition into multipotent progenitor cells that transition into tripotent, then bipotent, then unipotent progenitor cells and finally develop into the differentiated parenchymal and stromal cell types (FIG. 1).

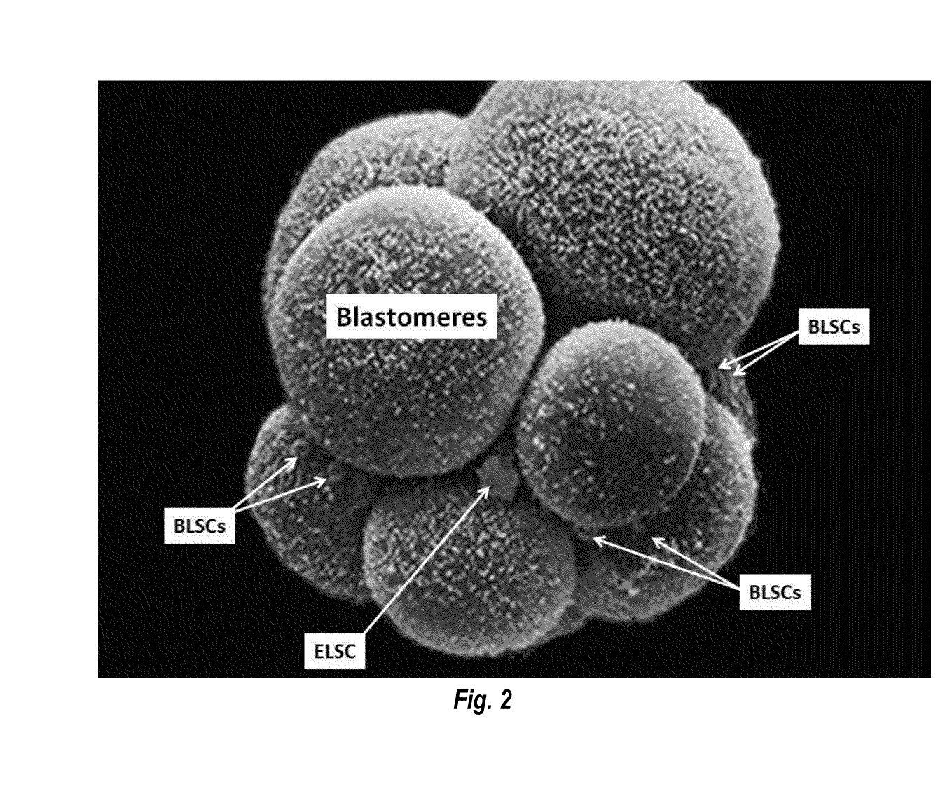

Remarkably, while the vast majority of developing cells transition through that sequence of developmental and differentiation events, a few cells become reserve precursor cells (FIG. 2) that provide for continual maintenance and repair of the organism. Known reserve precursor cells located within the postnatal individual include totipotent blastomeric-like stem cells, pluripotent epiblast-like stem cells, ectodermal germ layer lineage stem cells, endodermal germ layer lineage stem cells, the mesodermal germ layer lineage stem cells, other more differentiated germ layer lineage stem cells, and various tissue-committed and cellular-committed progenitor cells [Young et al., 2001, 2004a; Young and Black, 2005a]. In recent years, particular interest has focused on early-stage primitive stem cells, especially embryonic stem cells (ESCs) and induced pluripotent stem cells (iPSCs), because of their plasticity to form multiple cell types and their apparent longevity in culture. [Thompson et al., 1998; Yamanaka. 2009].

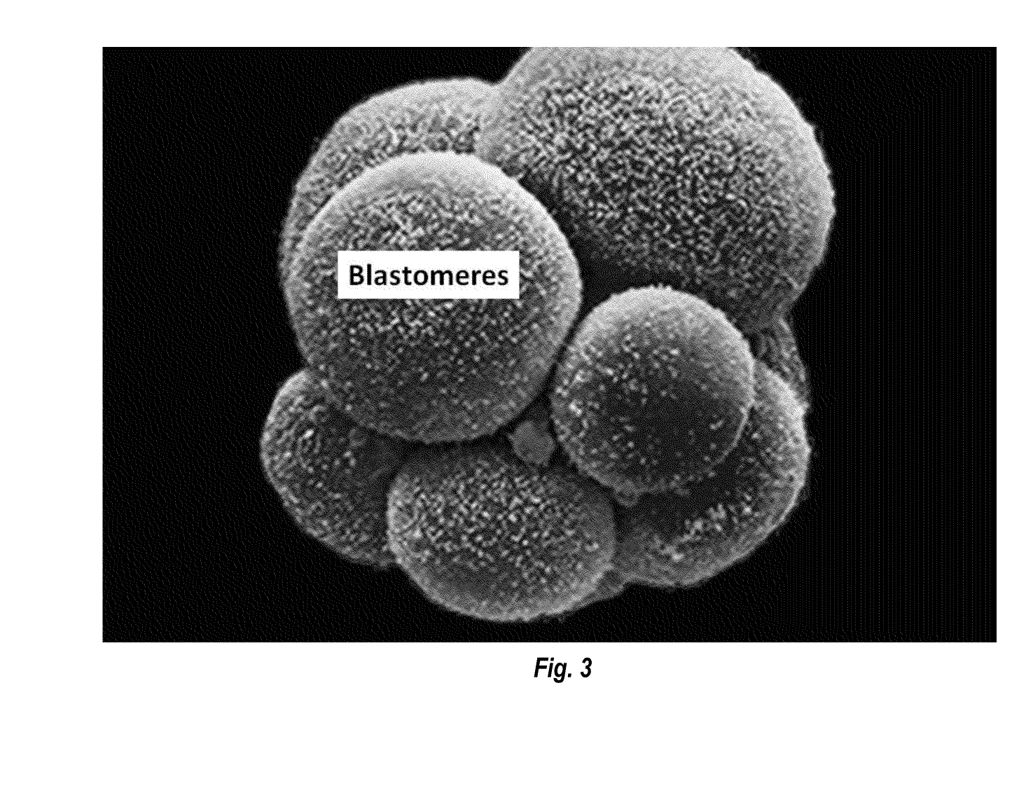

Embryonic stem cells (ESC) are relatively large uncommitted cells isolated from embryonic tissues. For example, ESCs have been isolated as disrupted blastomeres from the morula (FIG. 3), blastocyst, inner cell mass, and from cells of the gonadal ridges of mouse, rabbit, rat, pig, sheep, primate, and human embryos [Evans and Kauffman, 1981; Iannaccone et al., 1994; Graves and Moreadith, 1993; Martin, 1981; Notarianni et al., 1991; Thomson, et al., 1995; Thomson, et al., 1998; Shamblott, et al., 1998]. When injected into embryos, ESCs can give rise to all somatic lineages as well as functional gametes (i.e., sperm). ESCs typically spontaneously differentiate in serum-free defined medium in the absence of agents that inhibit differentiation (e.g., leukemia inhibitory factor). Further known embryonic stem cell preparations from embryoid tissue, post-morula tissue, blastocyst stage and pre-blastocyst stage were described in U.S. Pat. App. No. 2003/0175955, EP 1 176 189, WO 1997/020035, and WO 1995/016770, respectively. However, such cell preparations are isolated from an embryo, which is ethically controversial. Totipotent bovine embryonic stem cells have been reported in U.S. Pat. No. 6,107,543, and ungulate germ-line forming stem cells (possibly not totipotent) have been described in U.S. Pat. No. 6,703,209.

In still further known methods, pluripotent stem cells have been isolated from non-embryonic sources, including from umbilical cord matrix as described in U.S. Pat. App. No. 2003/0161818 and postnatal gonadal tissue as taught in WO 2002/031123. However, while such cells do not require destruction of an embryo and are therefore potentially of interest for human stem cells. However, the cells would be allogeneic in a stem cell transplant and the isolated stem cells have not been demonstrated to be totipotent.

Upon differentiation in vitro all or almost all of these cells express a wide variety of cell types, including gametes, as well as cells derived from the ectodermal, mesoderm, and endodermal germ layer lineages. Unfortunately, when currently known uncommitted embryonic stem cells are implanted into animals, they typically spontaneously differentiate in situ, forming teratomas. These tumors contain various types of cells and tissue derived from all three primary germ layer lineages [Thomson et al., 1988]. Therefore, while embryonic stem cells appear to have therapeutic potential in transplantation therapies their tendency to differentiate spontaneously in an uncontrolled manner places limitations on their usefulness.

Induced pluripotent stem (iPS) cells are generated from adult differentiated somatic cells by the insertion of embryonic genes [Yamanaka. 2009; Jalving and Shepers, 2009; Yamanaka and K. Takahashi, 2012]. The iPS cells were created to bypass the ethical and moral issues of dealing with embryonic and/or fetal tissues, since they were initially produced from adult differentiated somatic cells. Induced pluripotent stem cells express embryonic characteristics. They have capabilities for differentiation similar to embryonic stem cells, and are readily available for study. With respect to regenerative medicine, iPS cells are similar to embryonic stem cells. iPS cells spontaneously differentiate in culture in the absence of differentiation inhibitors. And like embryonic stem cells [Yamanaka. 2009], iPS cells form teratomas when placed in vivo in a naive undifferentiated state [Yamanaka and K. Takahashi, 2012]. Unfortunately, the production of teratomas reduces the utility of iPS cells as a source of stem cells for medical therapies. To avoid teratoma formation, iPS and embryonic stem cells must both be induced outside the body, i.e., ex vivo, to differentiate into the desired cell type before transplantation. Once induced, these cells lose their innate plasticity upon differentiation. This is the central dilemma for the use of embryonic stem cells or iPS cells in regenerative medicine. If one avoids differentiation ex vivo, teratomas result. If one differentiates these cells to avoid teratoma formation, then the resulting cells lose plasticity and gain a limited life-span.

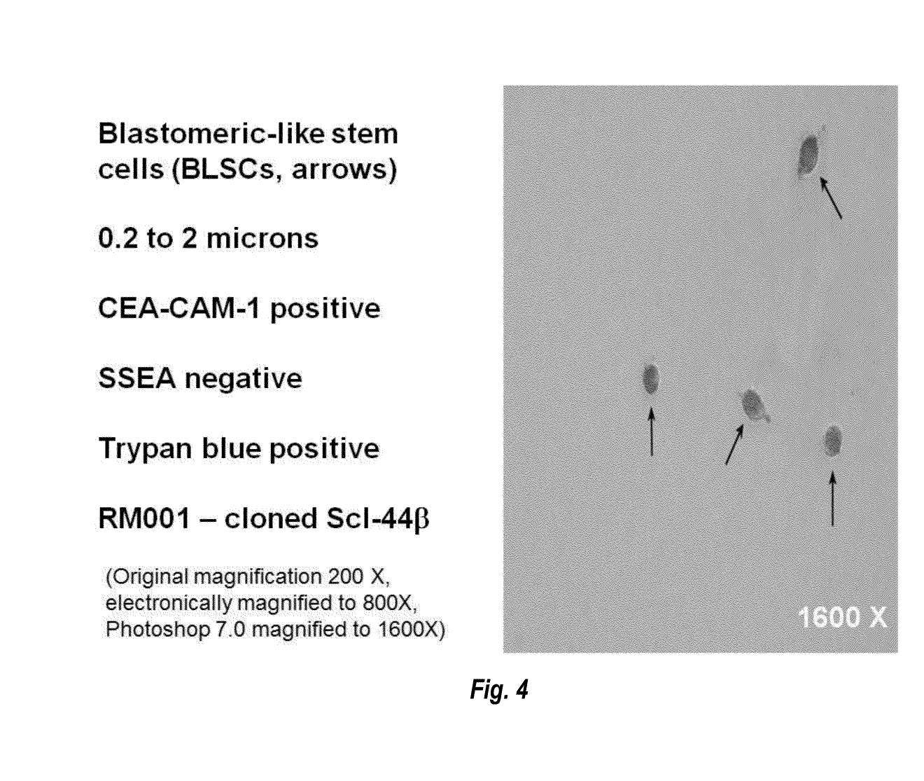

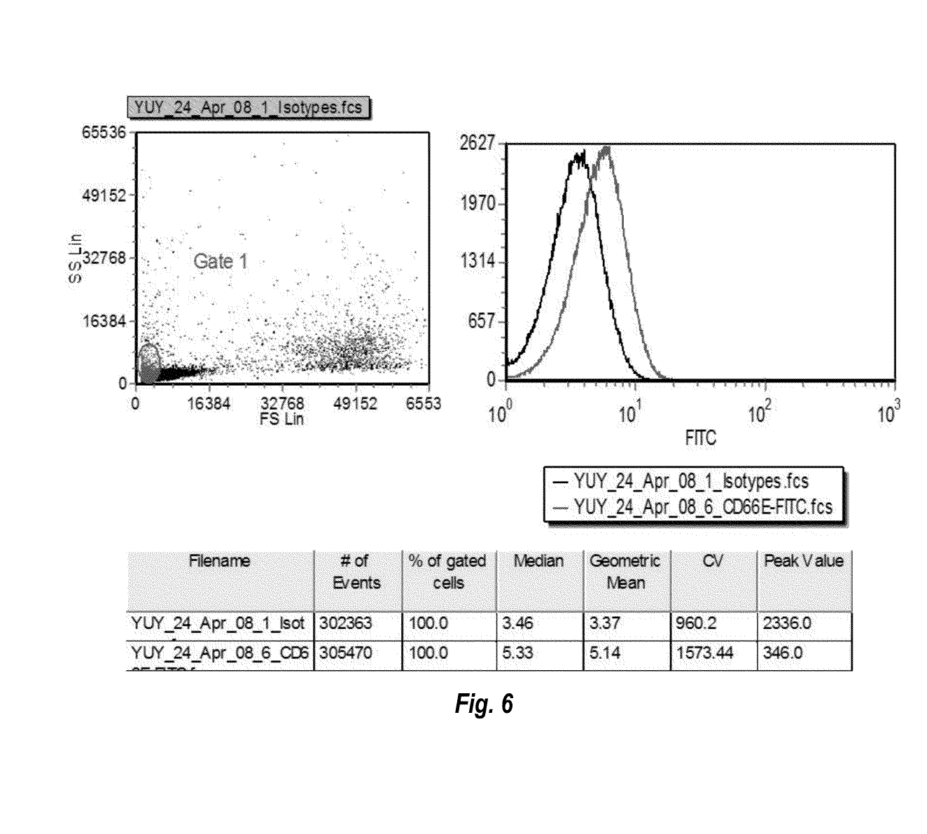

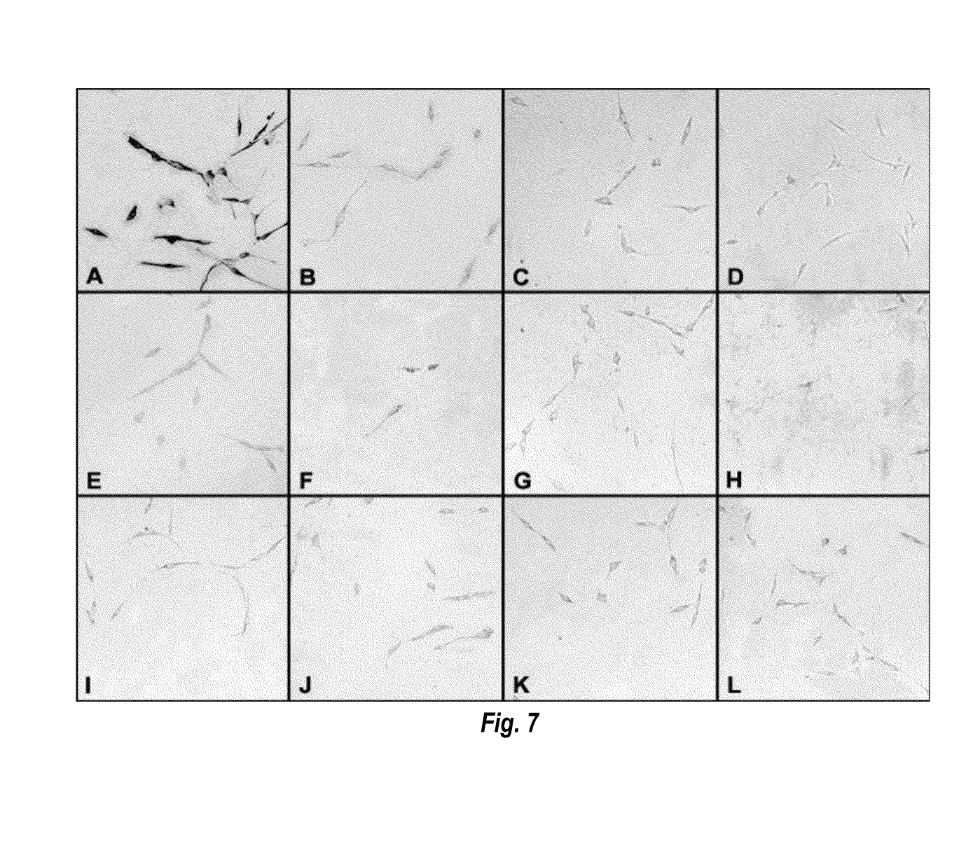

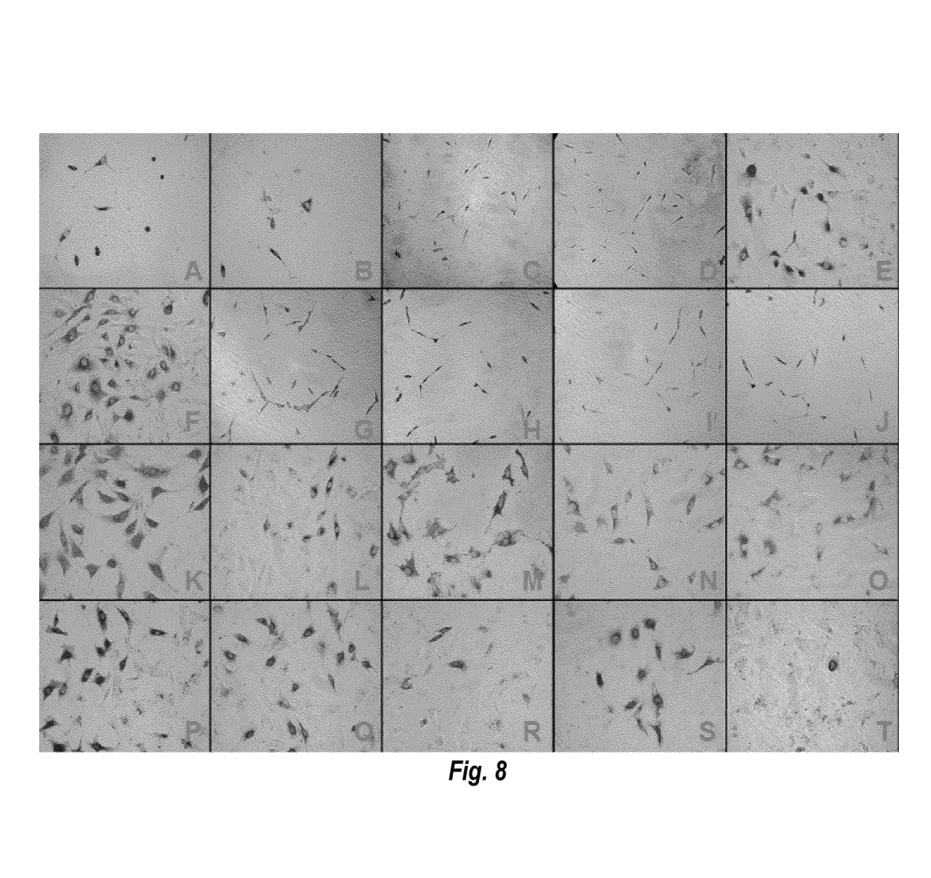

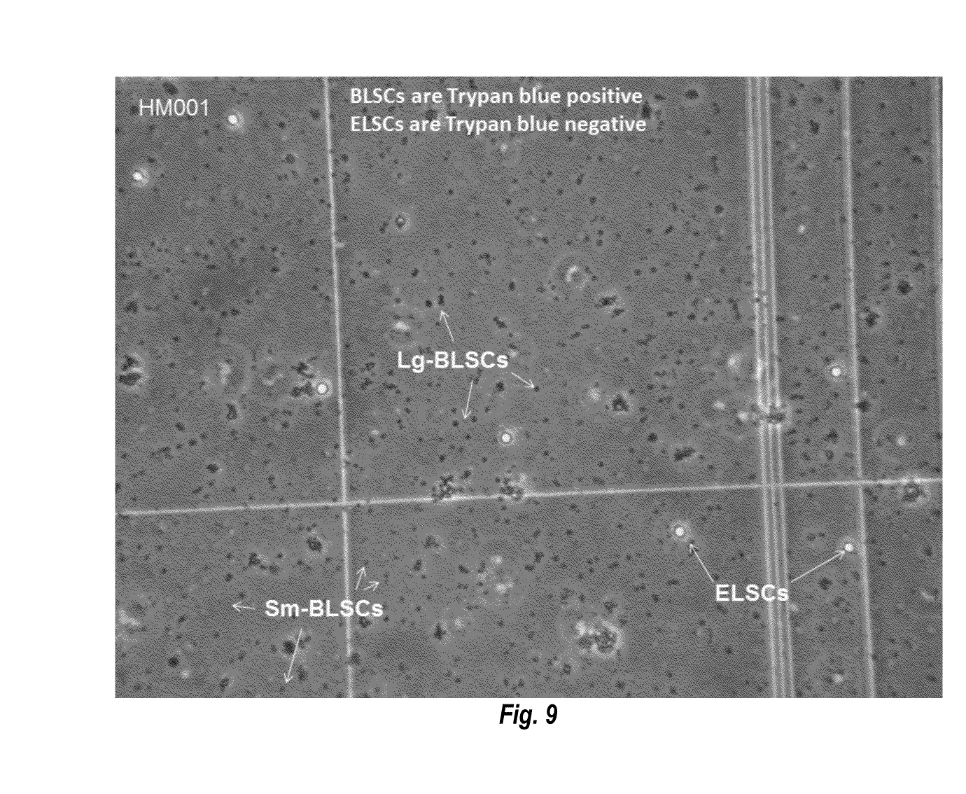

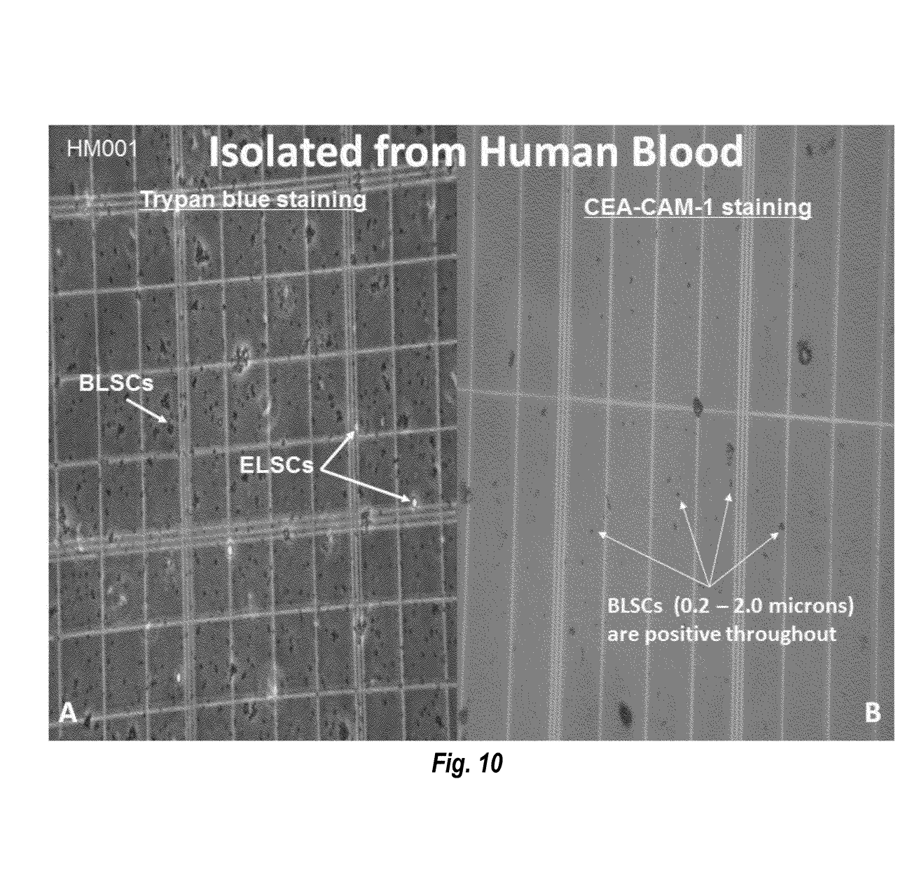

Adult blastomeric-like stem cells can be isolated from autologous or allogeneic individuals. They range in size from 2.0 microns to less than 0.2 microns in size (FIGS. 4,5), as determined by flow cytometry of living cells (FIG. 6). They have the potential to form all the somatic cells of the body (FIG. 7,8) [Young and Black, 2005a] including the germ cell, spermatogonia (FIG. 8; Table 1) [Young and Black, 2005a]. Because of their ability to form germ cells, adult blastomeric-like stem cells can be considered totipotent, and are thus referred to as adult totipotent blastomeric-like stem cells. These cells are Trypan blue positive (FIG. 9, Table 1). We attribute the positive Trypan blue staining to the lack of cell pumps to pump out the dye from these small cells that have minimal cytoplasm for containing cellular cytoplasmic machinery. Adult totipotent blastomere-like stem cells have a unique pattern of cell surface markers (Table 1) and express carcinoembryonic antigen-cell adhesion molecule-1 (CEA-CAM-1), carcinoembryonic antigen (CEA), (human) carcinoembryonic antigen (HCEA) and CD66e (human carcinoembryonic antigen) on its cell surface (FIG. 10) [Young et al., 2004a,b].

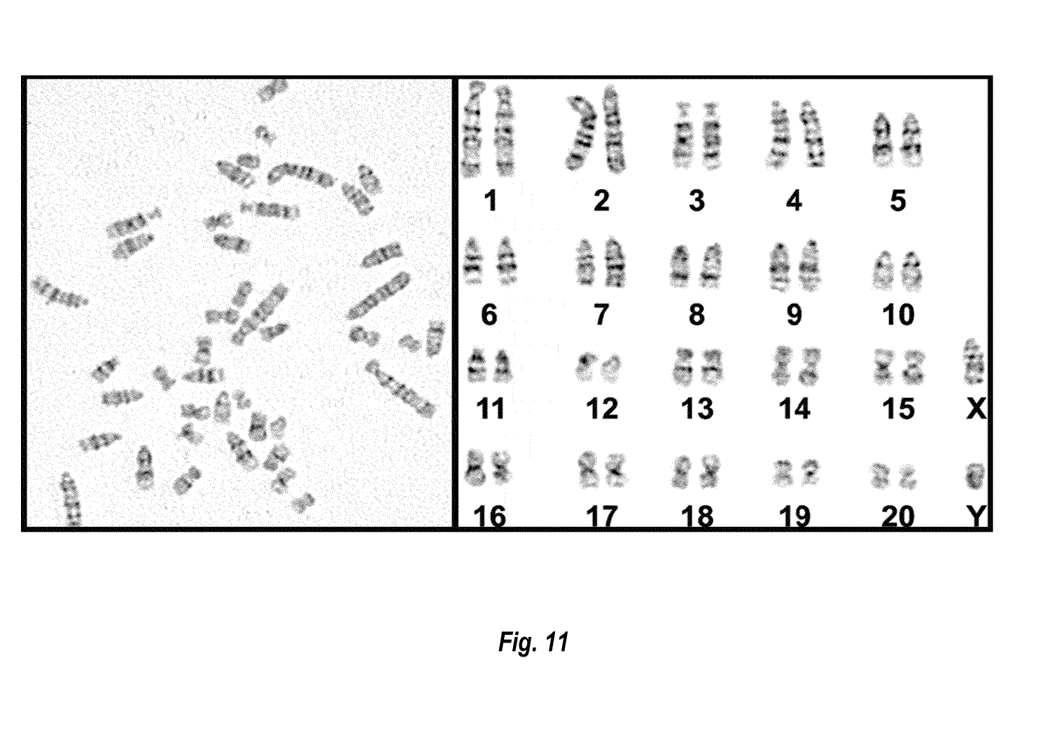

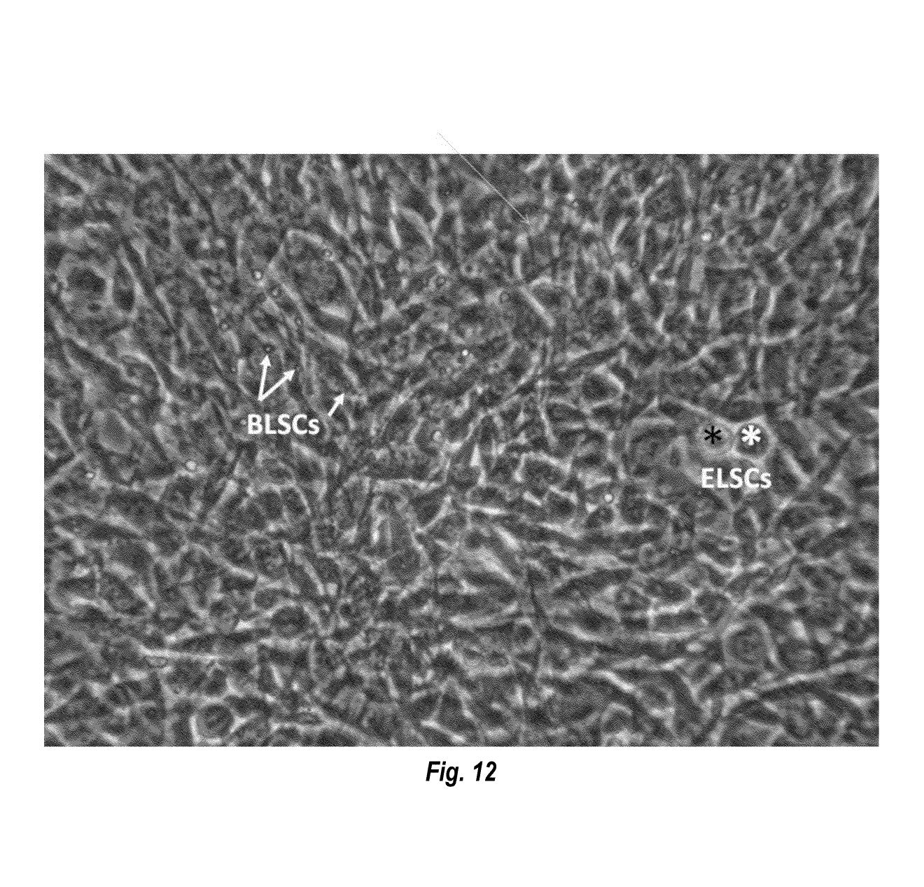

Adult blastomeric-like stem cells display a normal karyotype (FIG. 11). As long as adult totipotent blastomeric-like stem cells maintain their undifferentiated state, they have demonstrated extended capabilities for self-renewal far exceeding the preprogrammed replicative clock of progenitor cells and differentiative cells of rodent and human origin (Table 1) [Hayflick, Moorehead. 1961; Rohme 1981; Young and Black, 2005a]. Adult totipotent blastomeric-like stem cells do not exhibit contact inhibition at confluence, but continue to proliferate to form multiple adherent confluent layers of cells in culture (i.e., in vitro) (FIG. 12; Table 1) [Young and Black, 2005a]. The smaller-sized adult totipotent blastomeric-like stem cells, i.e., from 0.2 microns to less than 1 micron in size can also be grown in suspension cultures in vitro (Table 1). Adult totipotent blastomeric-like stem cells are responsive to any lineage-induction agent across all three primary germ layer lineages. It responds to brain-derived neurotrophic factor by forming cells belonging to the ectodermal lineage (FIG. 8). It responds to muscle morphogenetic protein and bone morphogenetic protein-2 by forming cells (myotubes and bone, respectively) belonging to the mesodermal lineage (FIG. 8). And it responds to hepatocyte growth factor by forming cells belonging to the endodermal lineage (FIG. 8; Table 1) [Young and Black, 2005a]. As long as the adult totipotent blastomeric-like stem cell remains lineage-uncommitted, it is unresponsive to progression agents like insulin, IGF-I or IGF-II that accelerate the time frame of expression for tissue specific phenotypic differentiation markers (FIG. 7; Table 1) [Young and Black, 2005a].

Adult totipotent blastomeric-like stem cells remain quiescent in vitro in a serum-free environment lacking proliferation agents, lineage-induction agents, progression agents, and/or inhibitory factors, such as recombinant human leukemia inhibitory factor (LIF), recombinant murine leukemia inhibitory factor (ESGRO), or recombinant human anti-differentiation factor (ADF) (similar to FIG. 7B-7L; Table 1) [Young et al., 2004a]. Cells with characteristics similar to adult totipotent blastomeric-like stem cells have been isolated from periosteum, perichondrium, nerve sheaths, adipose tissue, ligament, tendon, blood vessels, bone marrow, blood, and the connective tissue niches associated with trachea, lungs, esophagus, stomach, liver, intestines, spleen, brain, pancreas, kidney, urinary bladder, meninges, testis, tongue, thyroid, dermis, and skeletal muscle [Young, 2004; Young et al., 2004a,b; Young and Black, 2005a; Moore K L, Dalley A F, Ague A M. 2010; 90-92,100-103, 113].

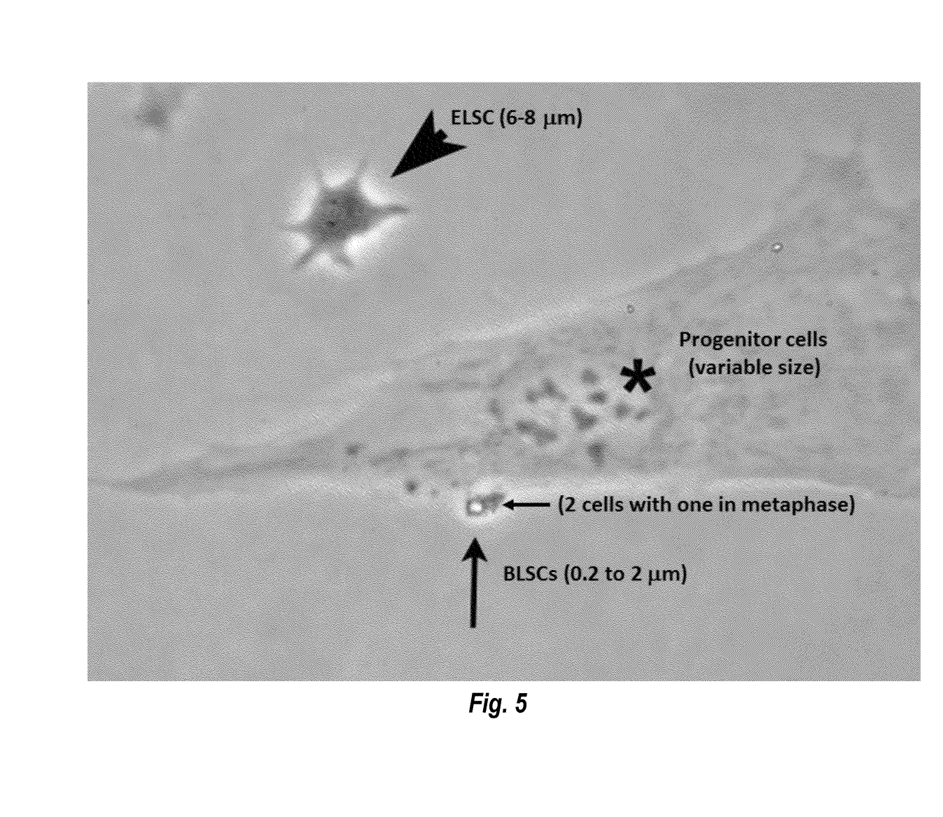

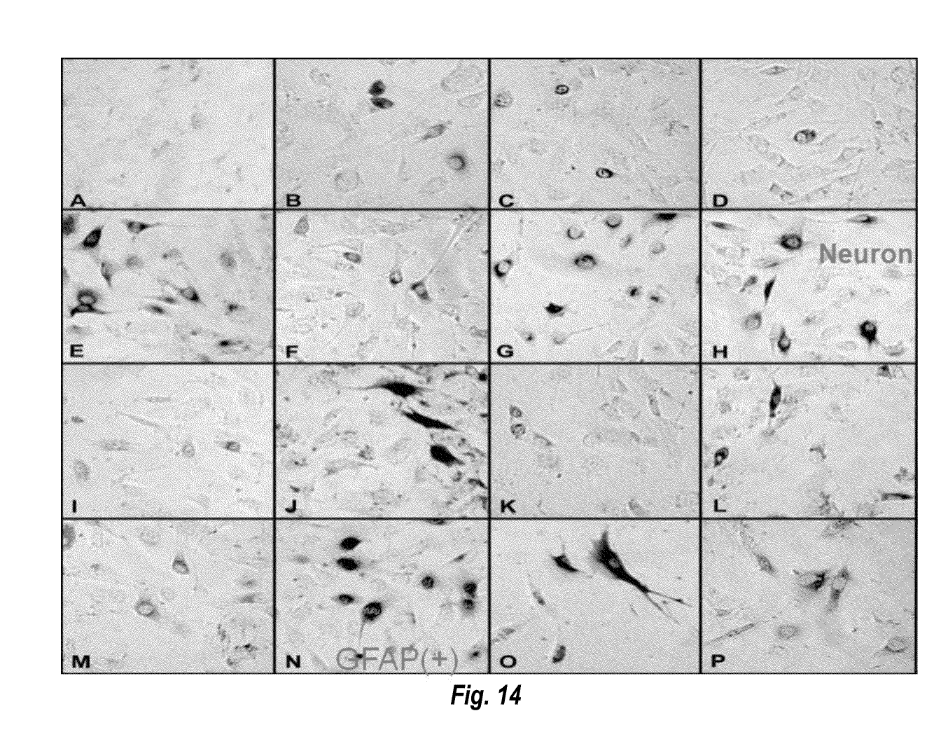

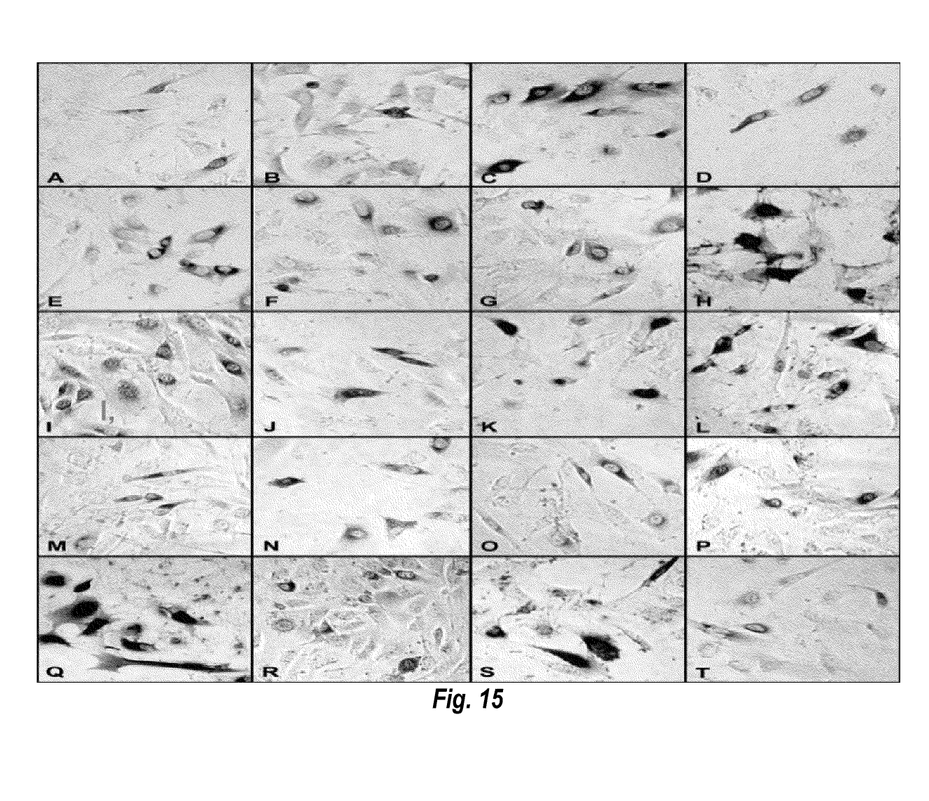

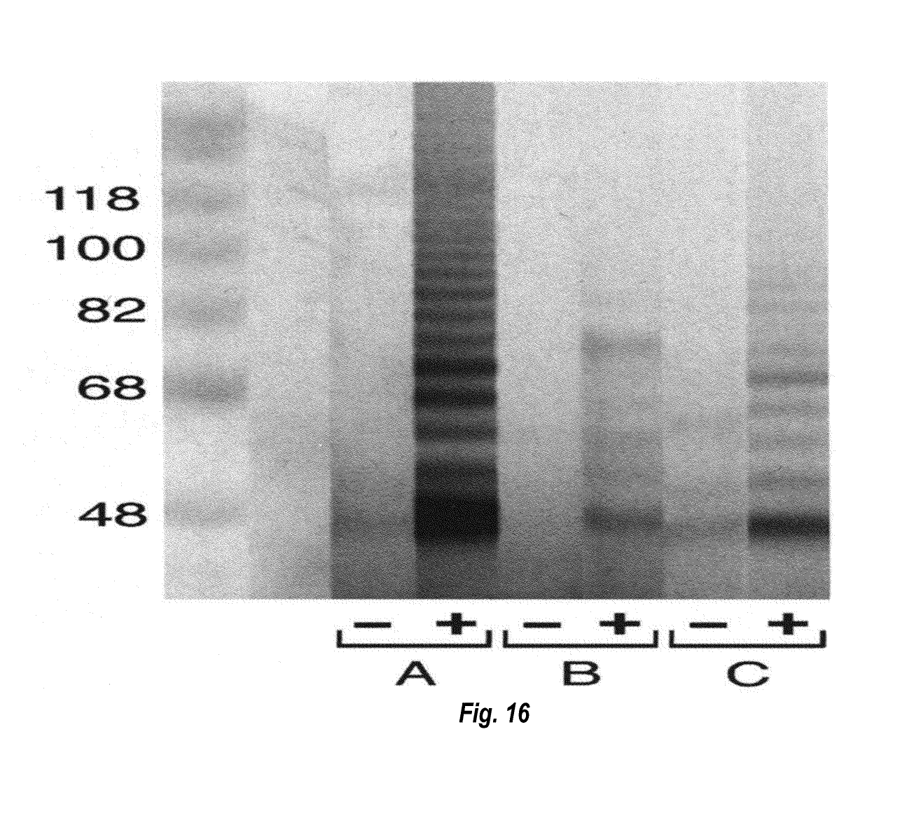

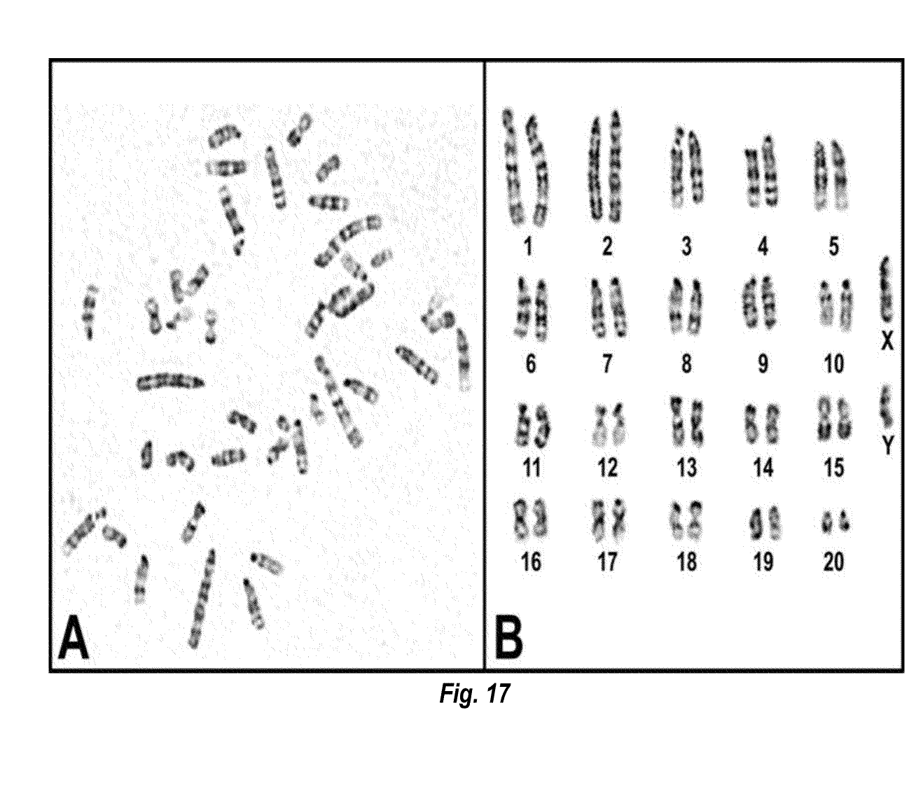

Adult pluripotent epiblast-like stem cells (ELSCs) are 6-8 microns in size as assessed by flow cytometry of living cells (Table 1, FIGS. 5,13) [Young et al., 1999]. ELSCs have the potential to form all the downstream somatic cells of the body (FIGS. 14,15) [Young et al., 2004a,b]. ELSCs will not form totipotent blastomeric-like stem cells, gametes (ovum and sperm) or their precursor cells (oogonia or spermatogonia) [Young and Black, 2005a]. Adult pluripotent epiblast-like stem cells are telomerase positive (FIG. 16, lane C+) [Young et al., 2004a,b] and have demonstrated extended capabilities for self-renewal far exceeding the preprogrammed replicative clock of progenitor cells and differentiative cells of rodent and human origin (Table 1) [Hayflick and Moorehead 1961; Rohme 1981; Young and Black, 2005]. Adult pluripotent epiblast-like stem cells have a unique pattern of cell surface markers (Table 1) [Young et al., 1999] and stain for stage-specific embryonic antigen (SSEA) (FIG. 8). This cell does not stain with Trypan blue and appears to glow white under phase contrast microscopy (FIGS. 9,10). This cell displays a normal karyotype (FIG. 17) [Henson et al., 2005]. Adult pluripotent epiblast-like stem cells require a growth substratum, but do not exhibit contact inhibition at confluence, rather continuing to proliferate to form multiple confluent layers of cells in vitro (FIG. 12; Table 1) [Moore and Dalley, 2010; Young, 2004; Young et al., 2004a;b; Young and Black, 2005a,b;]. Adult pluripotent epiblast-like stem cells are responsive to any lineage-induction agent across all three primary germ layer lineages (FIGS. 14,15; Table 1) [Young et al., 2004a,b; Young and Black, 2005]. Adult pluripotent epiblast-like stem cells respond to brain-derived neurotrophic factor by forming cells belonging to the ectodermal lineage (FIG. 14) [Young, 2004; Young and Black, 2005a,b, Young et al., 1998, 2004a,b, 2005a,b]. Adult pluripotent epiblast-like stem cells respond to muscle morphogenetic protein and bone morphogenetic protein-2 by forming cells (myoblasts and osteocytes respectively) belonging to the mesodermal lineage (FIG. 15) [Young, 2004; Young and Black, 2005a,b, Young et al., 1998, 2004a,b, 2005a,b]. And they respond to hepatocyte growth factor by forming cells belonging to the endodermal lineage (FIG. 15) [Young, 2004; Young and Black, 2005a,b, Young et al., 1998, 2004a,b, 2005a,b]. As long as the adult pluripotent epiblast-like stem cell remains lineage-uncommitted, it is unresponsive to progression agents like insulin, IGF-I or IGF-II that accelerate the time frame of expression of tissue specific phenotypic differentiation markers (Table 1). Adult pluripotent epiblast-like stem cells remain quiescent in a serum-free environment lacking proliferation agents, lineage-induction agents, progression agents, and/or inhibitory factors, such as recombinant human leukemia inhibitory factor (LIF), recombinant murine leukemia inhibitory factor (ESGRO), or recombinant human anti-differentiation factor (ADF) (Table 1) [Young, 2004; Young and Black, 2005a,b, Young et al., 1998, 2004a,b, 2005a,b]. Cells with characteristics similar to adult pluripotent epiblast-like stem cells have been isolated from periosteum, perichondrium, nerve sheaths, adipose tissue, ligament, tendon, blood vessels, bone marrow, blood, and the connective tissue niches associated with trachea, lungs, esophagus, stomach, liver, intestines, spleen, brain, pancreas, kidney, urinary bladder, meninges, testis, tongue, thyroid, dermis, and skeletal muscle [Young et al., 1995, 1998, 1999, 2004a,b; Young and Black, 2005a; Moore and Dalley, 2010; Young, 2004; Henson et al., 2005; Vourc'h et al., 2004, 2005; Romero-Ramos et al., 2002; Seruya et al., 2004].

### Stem Cell Location

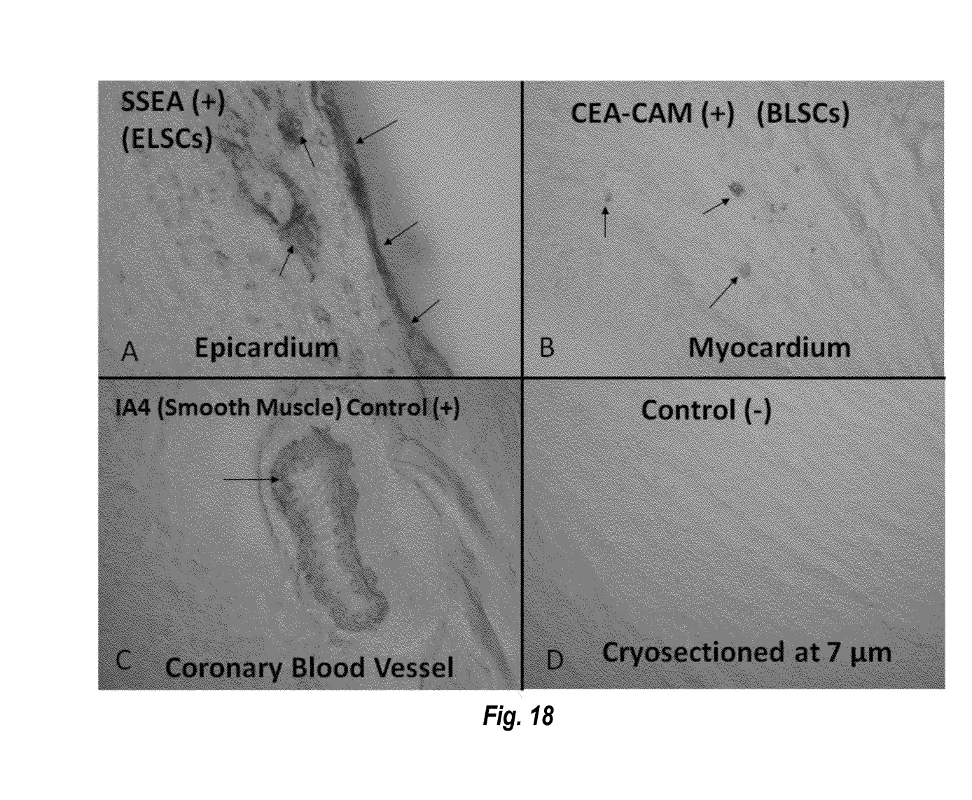

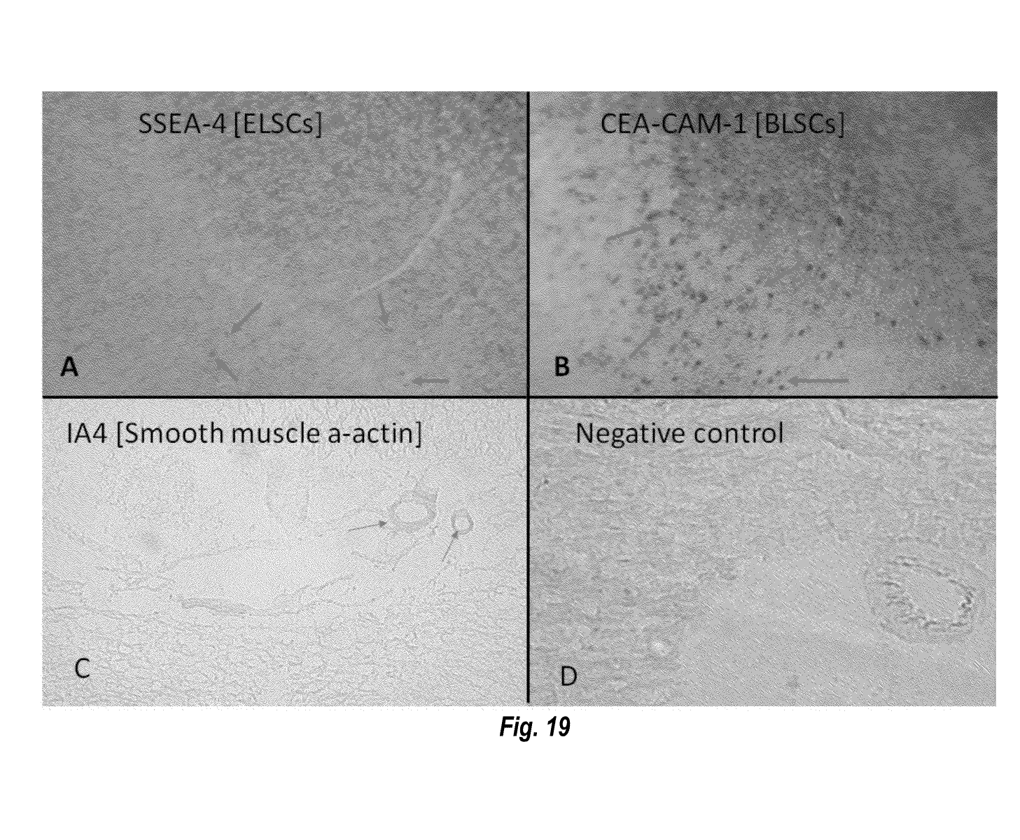

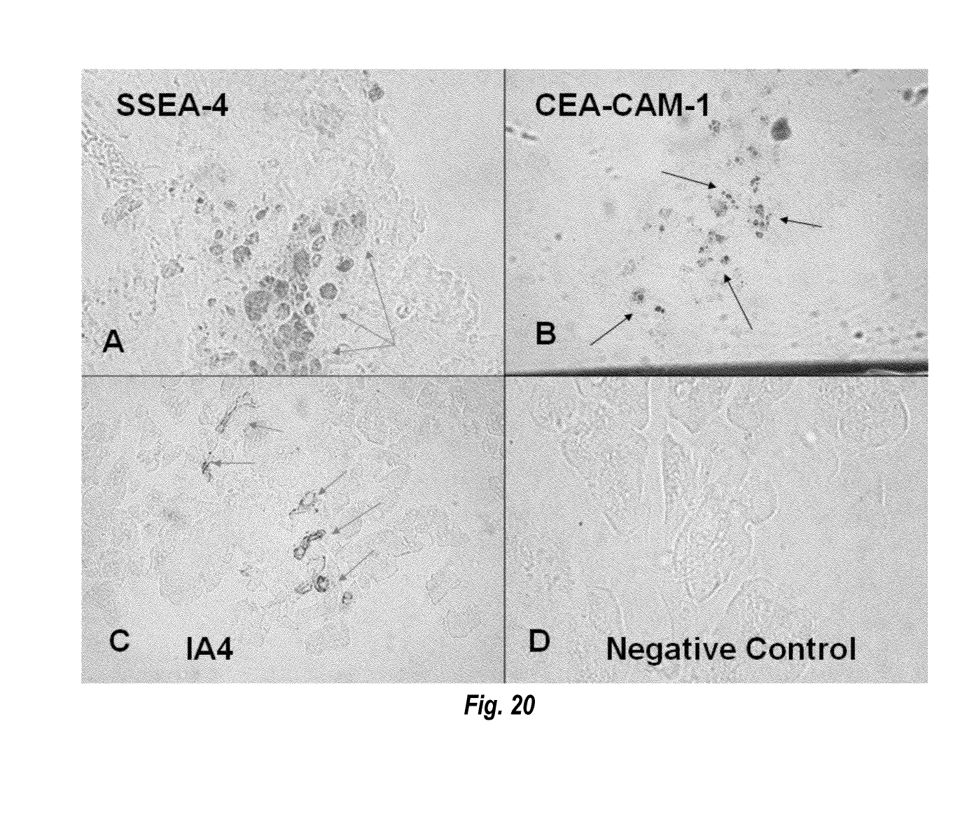

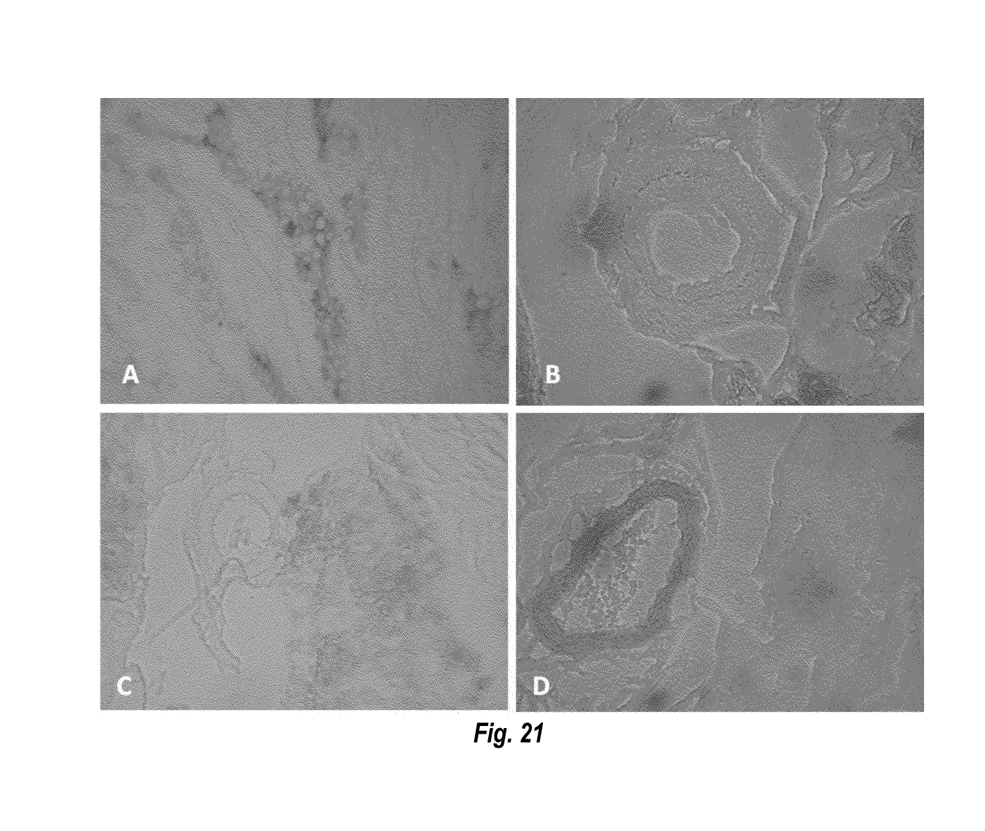

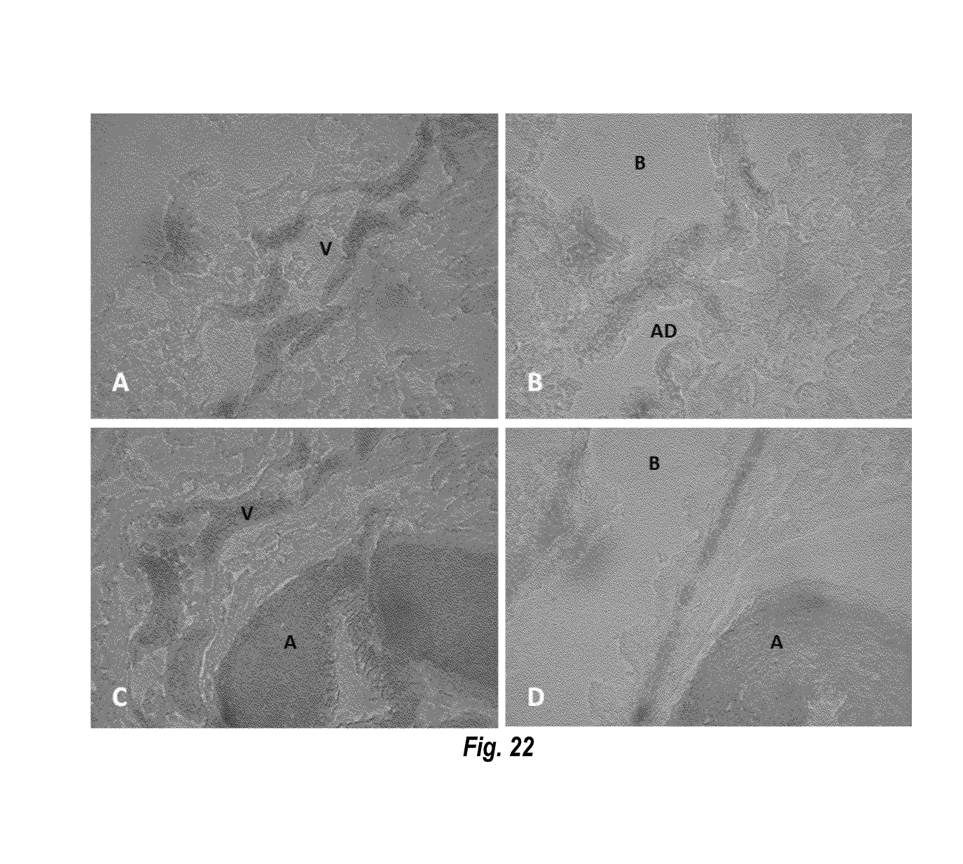

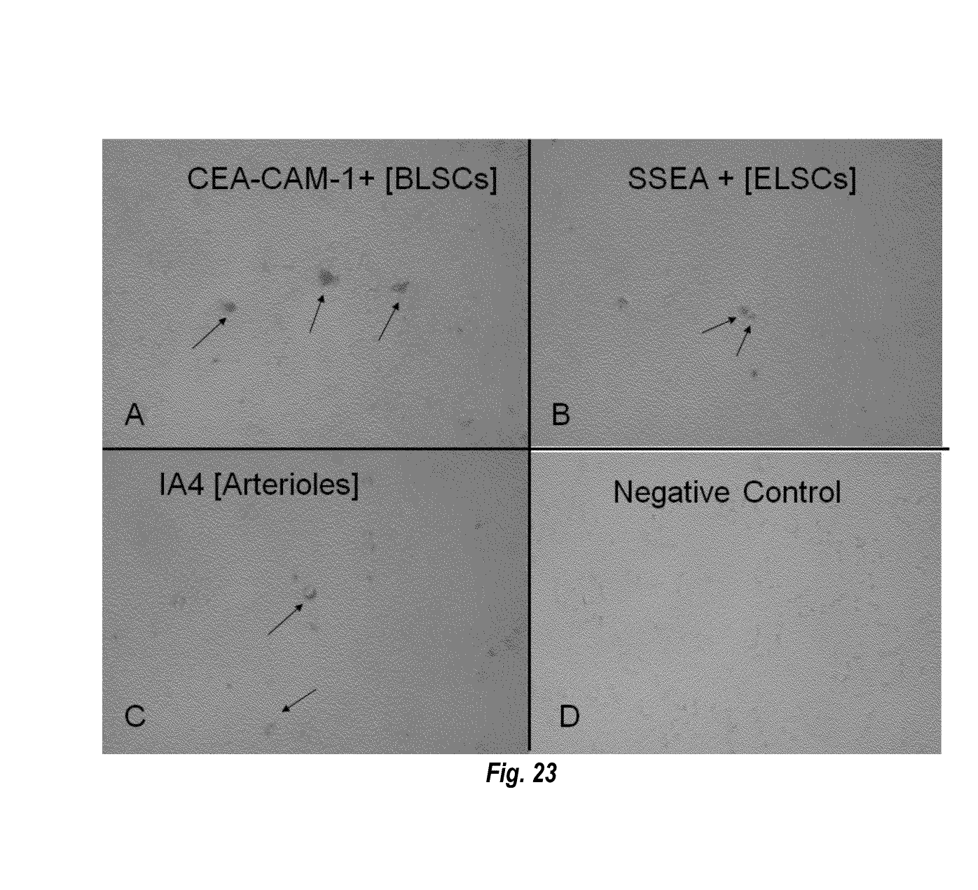

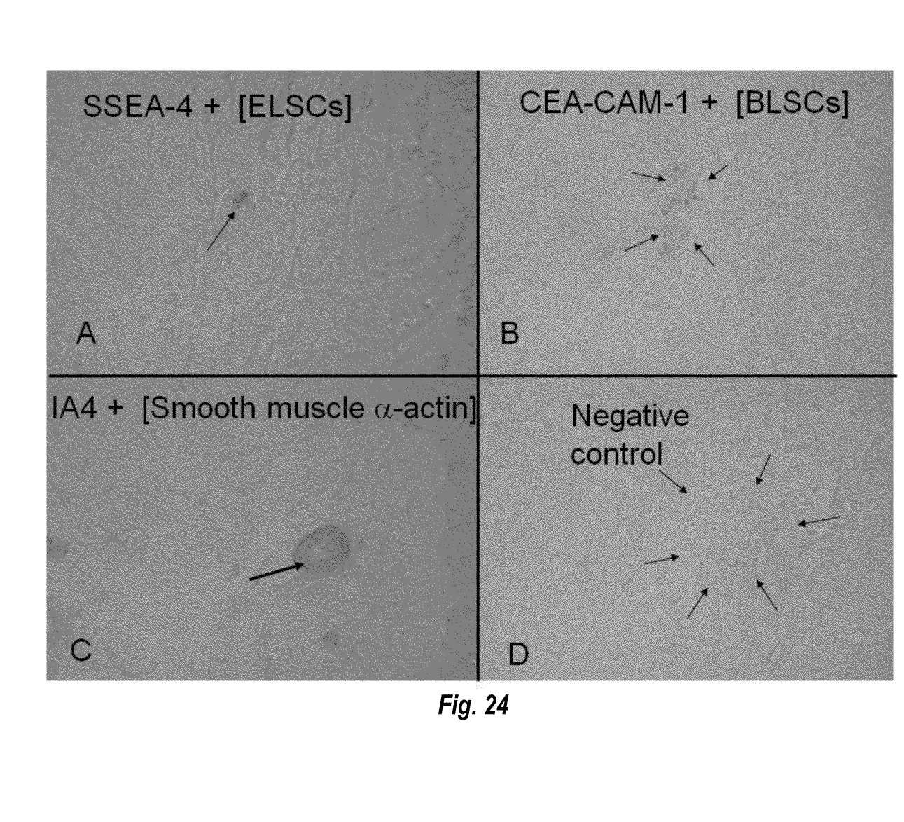

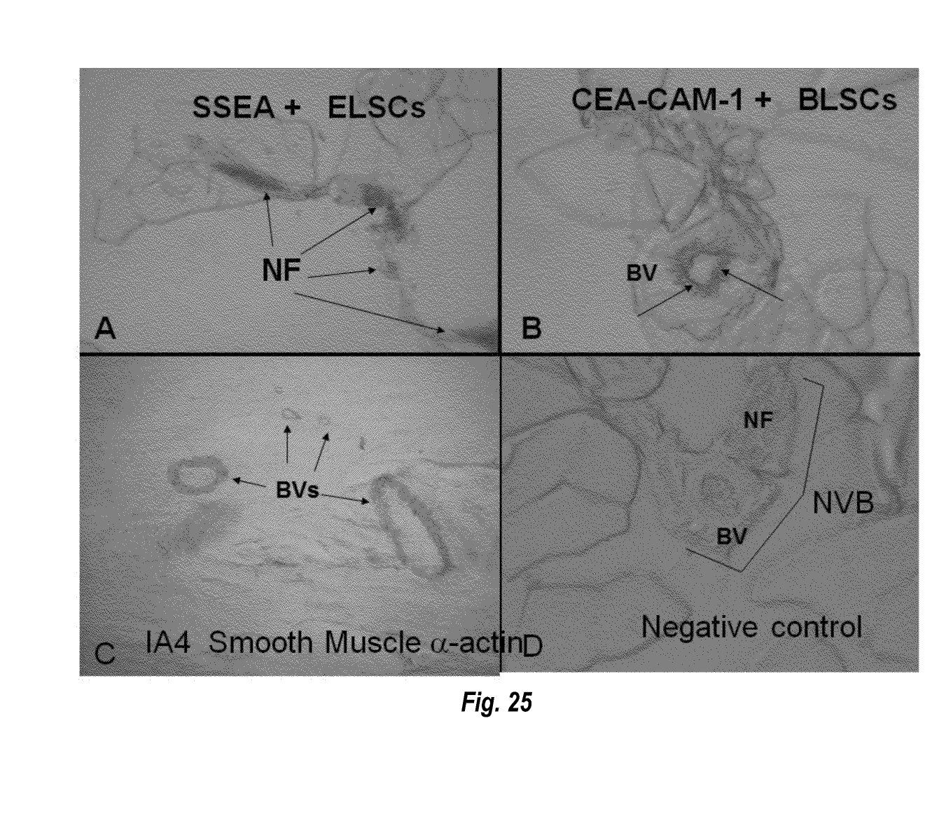

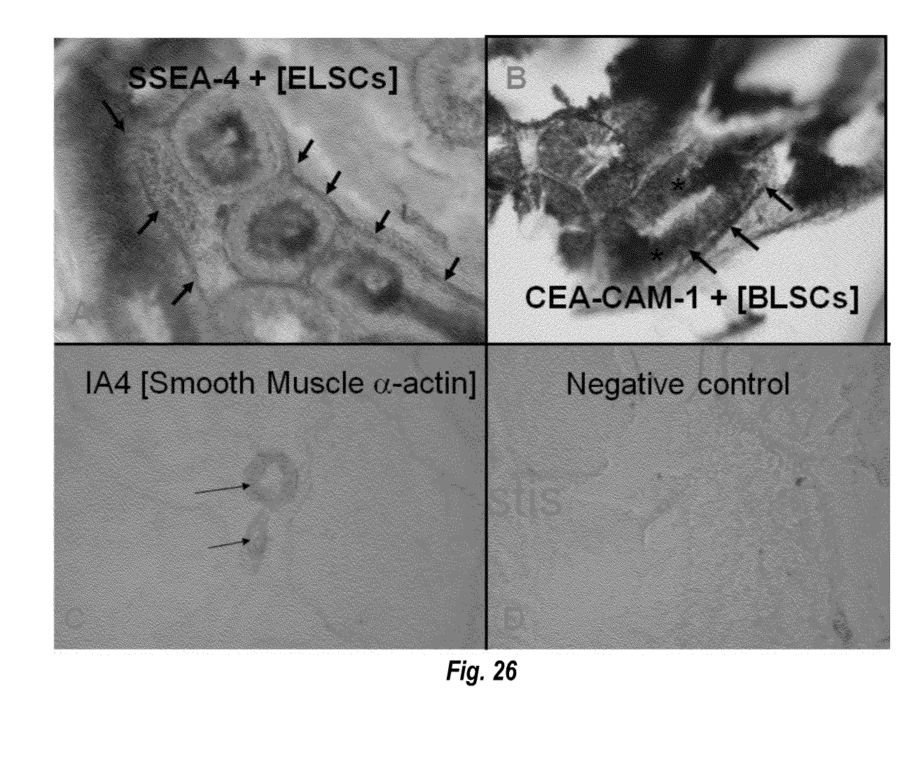

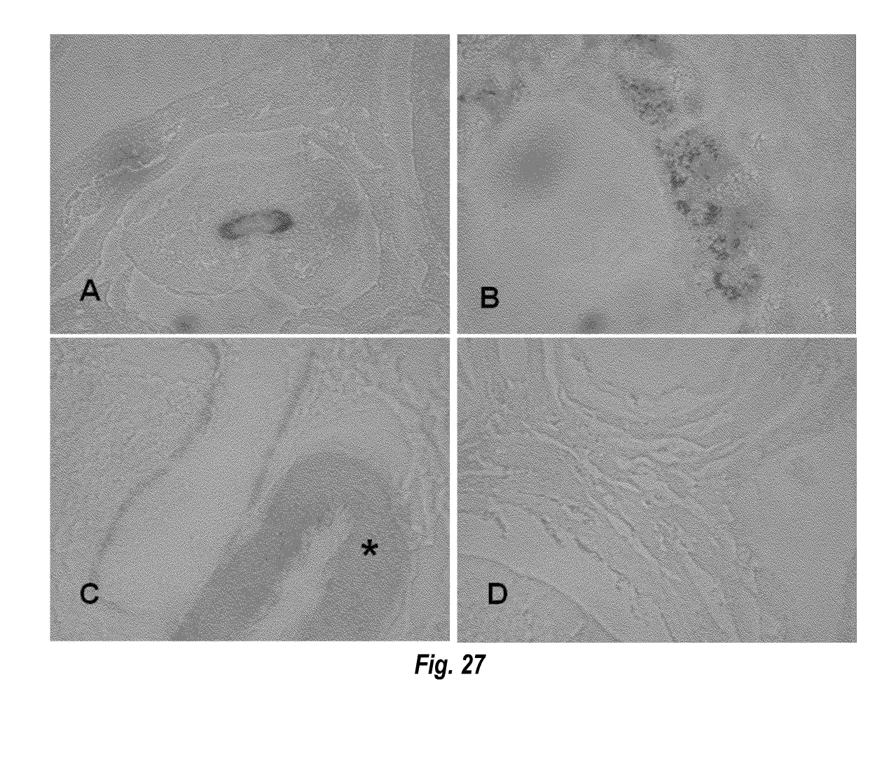

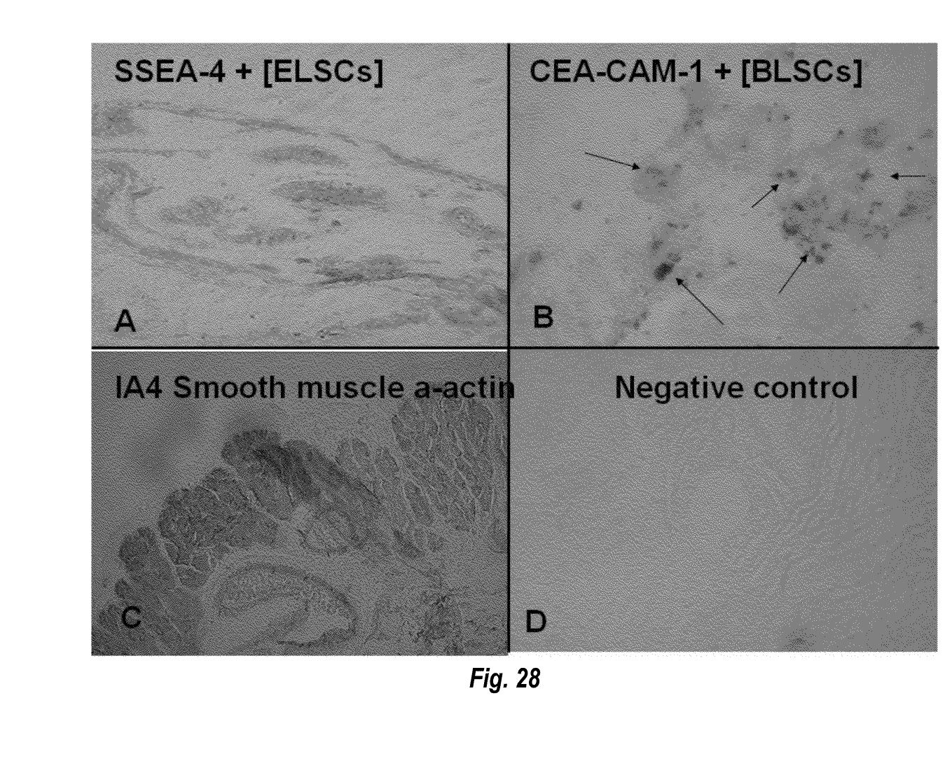

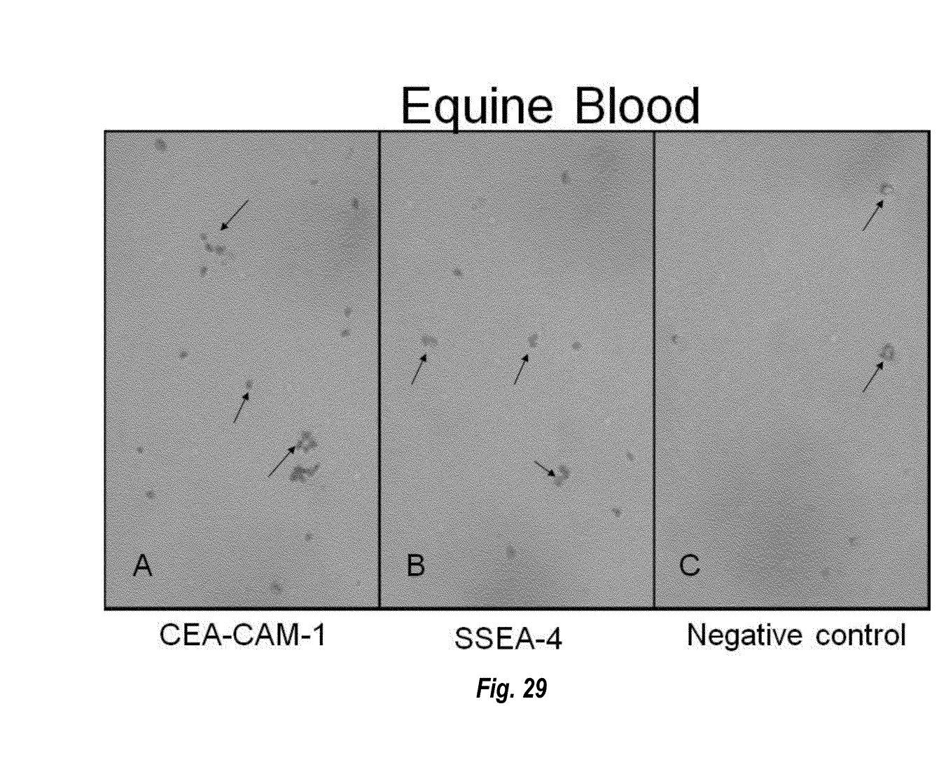

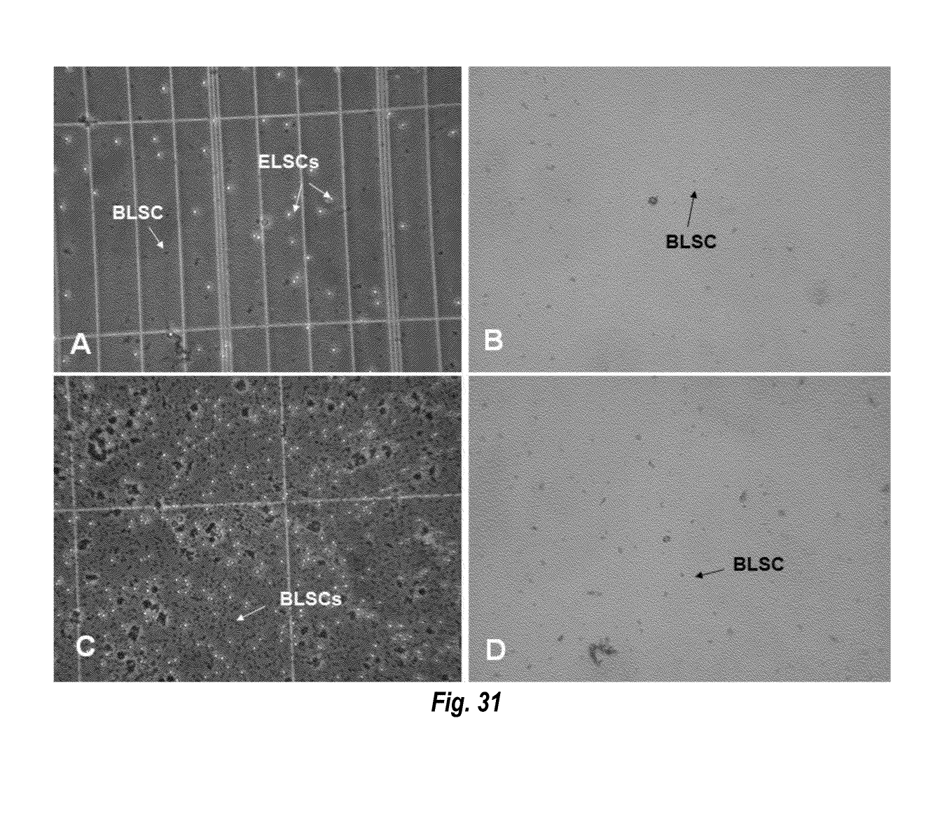

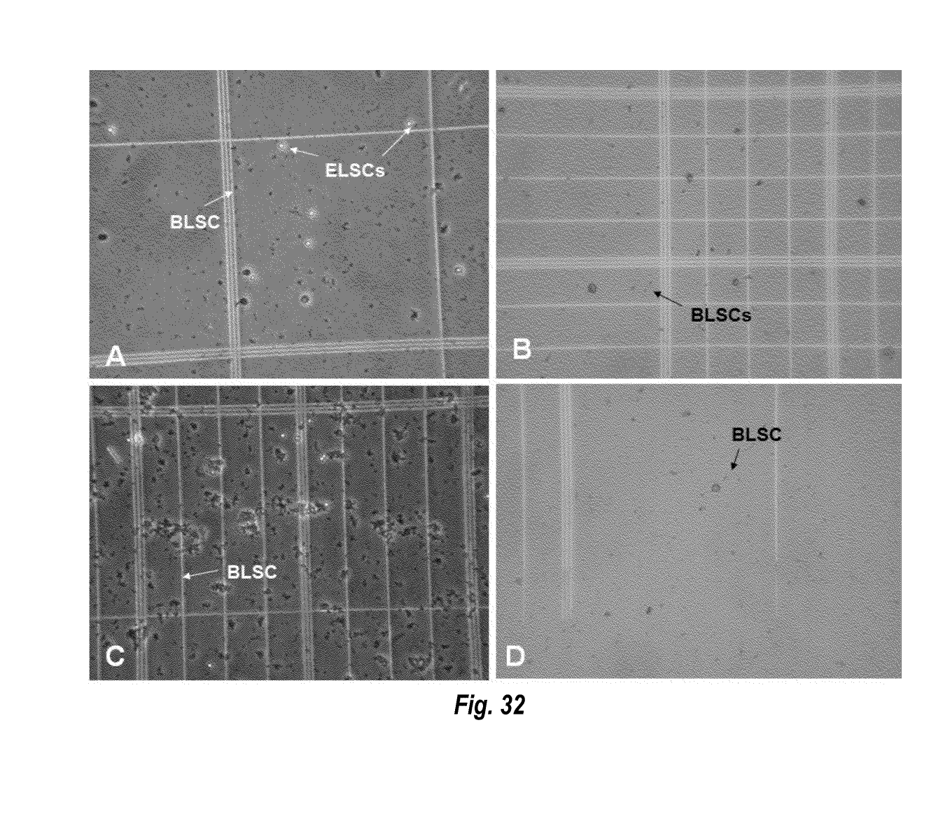

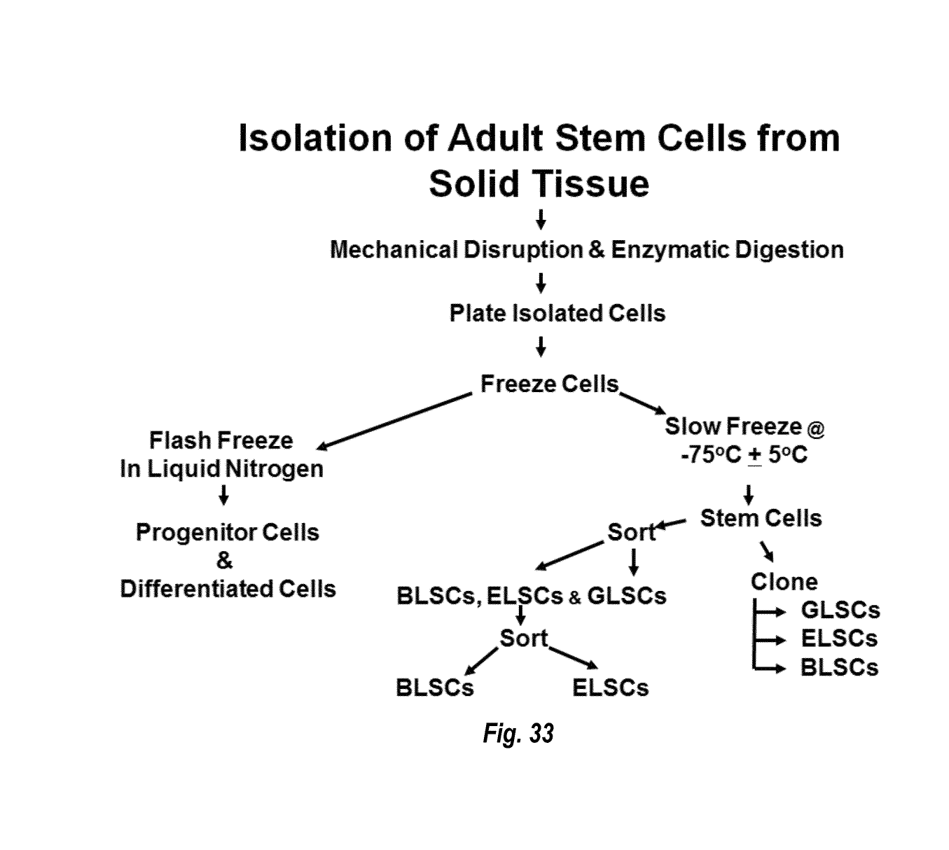

Studies have located the adult regenerative stem cells in the blood and in the supportive connective tissues of most organs. For example, both SSEA positive cells (ELSCs) and CEA-CAM-1 positive cells (BLSCs) were found in adult rat (RM1021) heart (FIG. 18), RM1021 cerebral hemisphere of the brain (FIG. 19), adult porcine pancreas (FIG. 20), dermis and hypodermis of the skin (FIG. 21), lung (FIG. 22), bone marrow (FIG. 23), porcine kidney (FIG. 24), skeletal muscle (FIG. 25), testis (FIG. 26), ovary (FIG. 27), and fallopian tube (FIG. 28)). We also found the adult regenerative pluripotent stem cells in blood: equine (FIG. 29), feline and canine (Slide 30), ovine (sheep) and caprine (goat) (FIG. 31), and bovine and equine blood (FIG. 32). Thus adult regenerative pluripotent stem cells were shown to be present in rat, pig, mouse, cat, dog, sheep, goat, cow, and horse. This was in conjunction with adult rat tissues and some human tissues examined by enzymatic digestion with collagenase and dispase, followed by cryopreservation, flash thawing frozen cells, sorting and/or cloning techniques (FIG. 33). The tissues examined were skeletal muscle, dermis of the skin, granulation tissue, periosteum, adipose tissue, perichondrium, nerve sheaths, ligament, tendon, elastic blood vessels (e.g., ascending aorta, arch of the aorta, brachiocephalic arterial trunk, common carotid arteries, subclavian arteries) blood vessels, muscular blood vessels (e.g., axillary artery, brachial artery, radial artery, ulnar artery, internal thoracic artery, superior epigastric artery, musculophrenic artery, pudendal artery, anterior tibial artery, posterior tibial artery), bone marrow, blood, trachea, lungs, esophagus, stomach, liver, intestines, spleen, brain, pancreas, kidney, urinary bladder, meninges, testis, tongue, and thyroid. The predominant species examined were rat and then human, although the stem cells were present in other species as well, i.e., cat, dog, sheep, goat, pig, cow, horse and human [Young et al., 2004a,b; Young and Black, 2005a,b; unpublished data].

### Stem Cell Propagation

Propagation of human and mammalian stem cells for transplantation therapies typically occurs ex vivo. The growth medium for most stem cells grown in culture is routinely supplemented with animal and/or human serum to optimize and enhance cell viability (Young et al., 1992, 1993, 1998b, 2001b; Young, 1999b; Young and Black, 2012; Pate et al, 1993; Rogers et al., 1995; Lucas et al., 1995, Warejcka et al., 1996; Dixson et al., 1996; Centeno et al., 2009). The constituents of serum include water, amino acids, glucose, albumins, immunoglobulins, and one or more bioactive agents. Potential bioactive agents present in serum include agents that induce proliferation, agents that accelerate phenotypic expression, agents that induce differentiation, agents that inhibit proliferation, agents that inhibit phenotypic expression, and agents that inhibit differentiation (Young et al., 1998, 2004b). Unfortunately, the identity(ies), concentration(s), and potential combinations of specific bioactive agents contained in different lots of serum is/are unknown (Young et al., 1998, 2004b). One or more of these unknown agents in serum have shown a negative impact on the isolation, cultivation, cryopreservation, and purification of lineage-uncommitted blastomere-like stem cells. Similarly, where feeder layers for stem cells were employed, contamination of stem cell cultures with feeder layer specific components, and especially viruses frequently occurs (Young et al., 2004b).

Alternatively, serum-free media are known for general cell culture, and selected pluripotent stem cells have been propagated in such medium containing a plurality of growth factors as described in US20050164380, US20030073234, U.S. Pat. No. 6,617,159, U.S. Pat. No. 6,117,675, or EP1298202.

### Stem Cell Isolation

Isolation of various types of progenitor cells, mesenchymal stem cells, epiblast-like stem cells, or blastomeric-like stem cells usually occurs by centrifugation, enzymatic digestion, cryopreservation, cell sorting, and/or cloning (FIGS. 33-40). [Young et al., 1991, 1992a, 1993, 1995, 2001a,b, 2004b, 2005; Vourc'h et al., 2004, 2005; Seruya et al., 2004; Henson et al., 2005]. If enzymatic procedures are utilized to remove extracellular matrix materials surrounding the stem cells then the stem cells need to be extensively washed to rid the stem cells of attached enzymes that may impede future developmental events. Even with extensive washing procedures post isolation, from either in vitro cultures of animal or human tissues, a large majority of the procedures utilizing enzymatic removal of extraneous material leave behind unwanted “baggage” on the surface of the stem cells.

### Stem Cell Transplantation

Multiple techniques have been used to transplant stem cells into animal models for tissue repair. Methods for cell suspensions include the use of small diameter hollow bore instruments (needles, cannulae) for intravenous infusion, direct needle injection, stereotactic injection, intrathecal injection, etc. and spraying directly on the tissue surface. Alternative methods have used placement within a bio-protective material, bio-protective/bio-degradable material or other such material that protects the cells from the immune system of the host while allowing it to function in its normal capacity [Young et al., 1989a, 1990c; Shoptaw et al., 1991; Bowerman et al., 1991].

Thus, while numerous compositions and methods for stem cells are known in the art, all or almost all of them suffer from one or more disadvantages. Therefore, there is still a need for improved stem cells, compositions, and reagents for their production, maintenance, and differentiation, and especially for ease of use for transplantation therapies.

Anyone skilled in the art will understand that this technology has the potential to treat ALL somatic body tissues involved in congenital malformations, trauma, disorders, and diseases of any of the systems in humans and other mammals. Those conditions include neurological, pulmonary, gastrointestinal, urinary, orthopedic, autoimmune, third degree burns, and any other anomaly dealing with cells within the body, EXCEPT the germ cells (sperm and ova) in the reproductive organs. Various objects, features, aspects and advantages of the present invention will become more apparent from the following detailed description of preferred embodiments of the invention.

### Experiments

The following descriptions and protocols are provided to give exemplary guidance to a person to make and use various aspects of the inventive subject matter presented herein. However, it should be appreciated that numerous modifications can be made without departing from the spirit of the present disclosure. Further contemplations, considerations, and experimental details are provided in WO 01/21767, U.S. Pat. App. Nos. 2003/0161817, and 2004/0033214, all of which are incorporated by reference herein.

### Solutions, Media, and Supplies

Bleach Solution: 0.5% Sodium hypochlorite (undiluted Clorox).

Disinfectant: The disinfectant of choice is Amphyl solution: 0.5% (v/v) in deionized water. In a 20 L carboy add 100 ml of Amphyl and then add 20 L of deionized water. However, 70% ethanol or other disinfectants not harmful to the cells may be utilized.

70% (v/v) Ethanol: Dilute 95% ethanol to 70% (v/v) with double deionized water. In a 500 ml glass media bottle, mix 368.4 ml of 95% ethanol with 131.6 ml of double deionized water. Store solution at ambient temperature.

70% (v/v) Isopropyl alcohol. Purchased from local Wal-Mart. Use at designated concentration of 70%.

Sterile 5M sodium hydroxide: Weigh out 20 g of sodium hydroxide granules (catalog #S318, Fisher Scientific, Pittsburgh, Pa.) and add to a glass media bottle. Very slowly add 100 ml of double deionized water to the sodium hydroxide granules. Once the sodium hydroxide is dissolved, filter sterilizes the solution through a 0.1 μm bottle top vacuum filter. Store solution at ambient temperature.

Sterile 5M hydrochloric acid: Measure 58.3 ml of double deionized distilled water and place in a 100-ml glass media bottle. Measure 41.7 ml of 12 M HCl (catalog #5619-02, VWR, JT5619-2, Bristol, Conn.) and very slowly add to water. Place cap on bottle and swirl gently to mix contents. Filter sterilizes the solution through a 0.1 μm bottle top vacuum filter. Store contents at ambient temperature.

0.4% Trypan Blue solution: Weigh out 0.2 g of Trypan blue (catalog #11618, Eastman Kodak Company, Rochester, N.Y.) and place in a sterile 100 ml glass media bottle. Under sterile conditions using a 25 ml pipette, add 50 ml of sterile Rinse buffer (catalog #MBC-ASB-REB-200-A001, Moraga Biotechnology Corp., Los Angeles, Calif.) containing 1% (or 1 ml of the 100×) antibiotic-antimycotic solution (catalog #15240-104, GIBCO), at pH 7.4. Swirl bottle gently to dissolve the Trypan blue powder. Filter sterilize the Trypan blue solution through a 0.2 μm bottle-top vacuum filter. Store this solution at ambient temperature.

Sterile Rinse Buffer with Ca⁺²/Mg⁺², pH 7.4: Under sterile conditions, take a fresh 500 ml bottle of sterile Rinse Buffer with Ca⁺²/Mg⁺² (catalog #MBC-ASB-REB-200-A001, Moraga Biotechnology Corp., Los Angeles, Calif.), discard 5 ml to bleach, and then add 5 ml of the 100× antibiotic-antimycotic solution (catalog #15240-104, GIBCO), for a final concentration of 1×. Invert the bottle a few times to mix the solution, and bring the pH to 7.4 using sterile 5M sodium hydroxide. Store solution at 4° C.

Sterile Release Buffer without Ca⁺²/Mg⁺², pH 7.4: Under sterile conditions, take a fresh 500 ml bottle of sterile Release Buffer without Ca⁺²/Mg⁺² (catalog #MBC-ASB-REB-200-A002, Moraga Biotechnology Corp.), discard 5 ml to bleach, and then add 5 ml of the 100× antibiotic-antimycotic solution (catalog #15240-104, GIBCO), for a final concentration of 1×. Invert the bottle a few times to mix the solution, and bring the pH to 7.4 using sterile 5M sodium hydroxide. Store solution at 4° C.

Sterile SFD-BLSC Rinse Buffer, Ca⁺²/Mg⁺², pH 7.4: Under sterile conditions, take a fresh 500 ml bottle of sterile serum-free-defined (SFD)-BLSC Rinse Buffer with Ca⁺²/Mg⁺² (catalog #MBC-ASB-REB-100-A001, Moraga Biotechnology Corp.), discard 5 ml to bleach, and then add 5 ml of the 100× antibiotic-antimycotic solution (catalog #15240-104, GIBCO), for a final concentration of lx. Invert the bottle a few times to mix the solution, and bring the pH to 7.4 using sterile 5M sodium hydroxide. Store solution at 4° C.

Sterile SFD-BLSC Release Buffer without Ca⁺²/Mg⁺², pH 7.4: Under sterile conditions, take a fresh 500 ml bottle of sterile serum-free defined (SFD) BLSC Release Buffer without Ca⁺²/Mg⁺² (catalog #MBC-ASB-REB-100-A002, Moraga Biotechnology Corp.) and discard 5.0 ml to bleach. Add 5 ml of the 100× antibiotic-antimycotic solution (catalog #15240-104, GIBCO) to the glass bottle (final concentration of 1×). Swirl to mix contents. pH to 7.4 with 5 M sodium hydroxide and/or 5 M hydrochloric acid. Store this solution at 4° C.

Dexamethasone solution, pH 7.4: This is typically made up in absolute ethanol (EtOH) because it is not soluble in water or media. Weigh out 0.039 gm of Dexamethasone (Dex, catalog #D-1756, Sigma) and add to 10 ml of absolute EtOH. This will make a 1×10⁻² M stock solution. Store this solution at −20° C. This is the most concentrated solution of Dex that can be made with complete solubility. Add 1 ml of the stock Dex solution made above to 9-ml Opti-MEM I medium with Glutamax. Aliquot 9 ml of this solution as 500 μl quantities in 2 ml cryovials and store at −20° C. Label these tubes as 1×10⁻⁶M Dex. Take the remaining 1 ml of 10⁻⁶M Dex and add to 9 ml of Opti-MEM I medium with Glutamax. Aliquot 9 mls and reserve 1 ml as before. Label these tubes as 1×10⁻⁷M Dex. Take the remaining 1 ml of 10⁻⁷M Dex and add to 9 ml of Opti-MEM I medium with Glutamax. Aliquot 9 mls and reserve 1 ml as before. Label these tubes as 1×10⁻⁸M Dex. Take the remaining 1 ml of 10⁻⁸M Dex and add to 9 ml of Opti-MEM I medium with Glutamax. Aliquot 9 mls and reserve 1 ml as before. Label these tubes as 1×10⁻⁹M Dex. Take the remaining 1 ml of 10⁻⁹M Dex and add to 9 ml of Opti-MEM I medium with Glutamax. Aliquot all 10 mls. Label these tubes as 1×10⁻¹⁰M Dex. These aliquots will bring 500 mls of media to the concentration of Dex labeled on the tube. Store the cryovials at −20° C.

Insulin solution, pH 7.4: Weight out 100 mg of Insulin (catalog #1-5500, Sigma) and add to a 15 ml conical. Under sterile conditions, add 5.0 ml of Opti-MEM I media with Glutamax to the conical. Invert the conical to dissolve the insulin. Filter sterilize twice using a 0.2 μm syringe filter, into a 15 ml conical first and then a 50 ml conical the second time. Measure volume present using a 5 ml pipette. Add enough Opti-MEM I media with Glutamax to bring the volume up to 15 mls. The final concentration will be approximately 1 mg/500 μl. Aliquot this solution into 1-ml cryovials, 500 μl each. Store the cryovials at −20° C. One cryovial will bring 500 mls of media up to the final concentration of 2 micrograms/ml.

Sterile Serum-Free Defined BLSC Media Supplements, pH 7.4: Under sterile conditions remove 7.975 ml from 500-ml bottle of sterile tissue culture medium of choice (e.g., EMEM, RPMI, Opti-MEM, or etc.) and discard to bleach. Add 7.975-ml aliquot of SFD-BLSC Media Supplements (catalog #MBC-ASB-MED-100-A001, Moraga Biotechnology Corp.) and swirl the bottle gently to mix contents. Remove 5.0 ml of solution and discard to bleach. Add 5 ml Antibiotic-Antimycotic solution. Swirl the bottle gently to mix contents and pH to 7.4. Store at 4° C.

Serum-Free Defined BLSC Basal Medium, pH 7.4: Under sterile conditions remove 5.0 mls from 500 ml bottle of Serum-Free Defined BLSC Basal Medium (catalog #MBC-ASB-MED-100-A002, Moraga Biotechnology Corp.) and discard to bleach. Add 5 ml Antibiotic-Antimycotic solution. Swirl the bottle gently to mix contents and pH to 7.4. Store at 4° C.

Propagation Supplement, pH 7.4: Under sterile conditions remove 6.0 mls from 500 ml bottle of medium supplemented with Serum-Free Defined BLSC Media Supplements (catalog #MBC-ASB-MED-100-A001, Moraga Biotechnology Corp.) and discard to bleach. Add 1.0 ml of Propagation Supplement (catalog #MBC-ASB-MED-100-A003, Moraga Biotechnology Corp.) and 5 ml of Antibiotic-Antimycotic solution. Swirl the bottle gently to mix contents and pH to 7.4. Store at 4° C.

Serum-Free Defined BLSC Propagation medium, pH 7.4: Under sterile conditions remove 5.0 mls from 500 ml bottle of Serum-Free Defined BLSC Propagation medium (catalog #MBC-ASB-MED-100-A006, Moraga Biotechnology Corp.) and discard to bleach. Add 5 ml Antibiotic-Antimycotic solution. Swirl the bottle gently to mix contents and pH to 7.4. Store at 4° C.

Serum-Free Defined BLSC Transport medium, pH 7.4: Under sterile conditions remove 15.0 mls from 500 ml bottle of Serum-Free Defined BLSC Transport medium (catalog #MBC-ASB-MED-100-A004, Moraga Biotechnology Corp.) and discard to bleach. Add 15 ml Antibiotic-Antimycotic solution. Swirl the bottle gently to mix contents and pH to 7.4. Store at 4° C.

Serum-Free Defined BLSC Cryopreservation medium, pH 7.4: Under sterile conditions, take a fresh 100 ml bottle of Serum-Free Defined BLSC Cryopreservation Medium, pH 7.4 (catalog #MBC-ASB-MED-100-A005, Moraga Biotechnology Corp.). Remove 1.0 ml of medium and discard to bleach. Add 1 ml Antibiotic-Antimycotic solution. Swirl the bottle gently to mix contents and pH to 7.4. Store at 4° C.

General Induction medium, pH 7.4: Serum-Free Defined BLSC Propagation Medium, pH 7.4, containing 10⁻⁸ M dexamethasone, 2 μg/ml insulin, 5% SS9, and 10% SS12. Under sterile conditions, take a fresh 500 ml bottle of SFD-BLSC Propagation medium (catalog #MBC-ASB-MED-100-A006, Moraga Biotechnology Corp.) remove 83 ml of medium and place into a sterile 100-ml bottle. Add 500 μl aliquot of insulin, 500 μl aliquot of dexamethasone, 5-ml of SS9 (catalog #H7889, Sigma), and 10-ml of SS12 (catalog #FB-01, Omega Scientific, Tarzana, Calif.). Swirl the bottle gently to mix solutions, pH to 7.4 and store at 4° C.

Ectodermal Induction medium, pH 7.4: Serum-Free Defined BLSC Propagation medium, containing 10⁻⁸ M dexamethasone, 2 μg/ml insulin, and 15% SS12, pH 7.4. Under sterile conditions, take a fresh 500 ml bottle of Serum-Free Defined BLSC Propagation medium, pH 7.4, and remove 83 ml of medium and place into a sterile 100-ml bottle. Add 500 μl aliquot of insulin, 500 μl aliquot of dexamethasone, 15-ml of SS12 (catalog #FB-01, Omega Scientific, Tarzana, Calif.). Swirl the bottle gently to mix solutions and store at 4° C.

Mesodermal Induction medium, pH 7.4: Serum-Free Defined BLSC Propagation medium, containing 10⁻⁸ M dexamethasone, 2 μg/ml insulin, and 10% SS9, pH 7.4. Under sterile conditions, take a fresh 500 ml bottle of Serum-Free Defined BLSC Propagation medium, pH 7.4, and remove 83 ml of medium and place into a sterile 100-ml bottle. Add 500 μl aliquot of insulin, 500 μl aliquot of dexamethasone, 10-ml of SS9 (catalog #H7889, Sigma). Swirl the bottle gently to mix solutions and store at 4° C.

Endodermal Induction medium, pH 7.2: Serum-Free Defined BLSC Propagation medium, containing 10⁻⁸ M dexamethasone, 2 μg/ml insulin, and 15% SS12, pH 7.4. Under sterile conditions, take a fresh 500 ml bottle of Serum-Free Defined BLSC Propagation medium, pH 7.4, and remove 83 ml of medium and place into a sterile 100-ml bottle. Add 500 μl aliquot of insulin, 500 μl aliquot of dexamethasone, 10-ml of SS12 (catalog #FB-01, Omega Scientific, Tarzana, Calif.). pH to 7.2 with 6 M HC1. Swirl the bottle gently to mix solutions and store at 4° C.

SFD-Tissue Release Solution, pH 7.4: SFD-Tissue Release Solution (catalog #MBC-ASB-RED-100-A003, Moraga Biotechnology Corp.), store the tubes at −20° C. until needed. Just before use, thaw, remove 1% solution and discard to bleach. Add 1% antibiotic-antimycotic solution and pH to 7.4.

SFD-Cell Release/Activation solution, pH 7.4: Under sterile conditions, take a fresh 500 ml bottle of SFD-Cell Release/Activation Solution (catalog #MBC-ASB-RED-100-A004, Moraga Biotechnology Corp.), remove 5.0 ml of solution and discard to bleach. Add 5 ml Antibiotic-Antimycotic solution. Swirl the bottle gently to mix contents and pH to 7.4. Store at 4° C.

SFD-Cell Release/Activation Inhibitor Solution, pH 7.4: Under sterile conditions, take a fresh 500 ml bottle of SFD-Cell Release/Activation Solution Inhibitor (catalog #MBC-ASB-RED-100-A005, Moraga Biotechnology Corp.), remove 5.0 ml of solution and discard to bleach. Add 5 ml Antibiotic-Antimycotic solution. Swirl the bottle gently to mix contents and pH to 7.4. Store at 4° C.

SFD-BLSC-MACS buffer, pH 7.2: Under sterile conditions, take a fresh 500 ml bottle of SFD-BLSC-MACS buffer (catalog #MBC-ASB-RED-100-A006, Moraga Biotechnology Corp.), remove 5.0 ml of solution and discard to bleach. Add 5 ml Antibiotic-Antimycotic solution. Swirl the bottle gently to mix contents and pH to 7.2. Store at 4° C.

Adult Stem Cell Coated culture vessels: 75 cm² flasks (catalog #MBC-ASB-MSD-900-A006, Moraga Biotechnology Corp.), 25 cm² flasks (catalog #MBC-ASB-MSD-900-A007, Moraga Biotechnology Corp.), 6-well plates (catalog #MBC-ASB-MSD-900-A008, Moraga Biotechnology Corp.), 24-well plates (catalog #MBC-ASB-MSD-900-A009, Moraga Biotechnology Corp.), 48-well plates (catalog #MBC-ASB-MSD-900-A010, Moraga Biotechnology Corp.), and 96-well plates (catalog #MBC-ASB-MSD-900-A011, Moraga Biotechnology Corp.).

### Animal Models of Tissue Replacement and Repair

### Ectodermal Lineage Repair: Parkinson's Disease

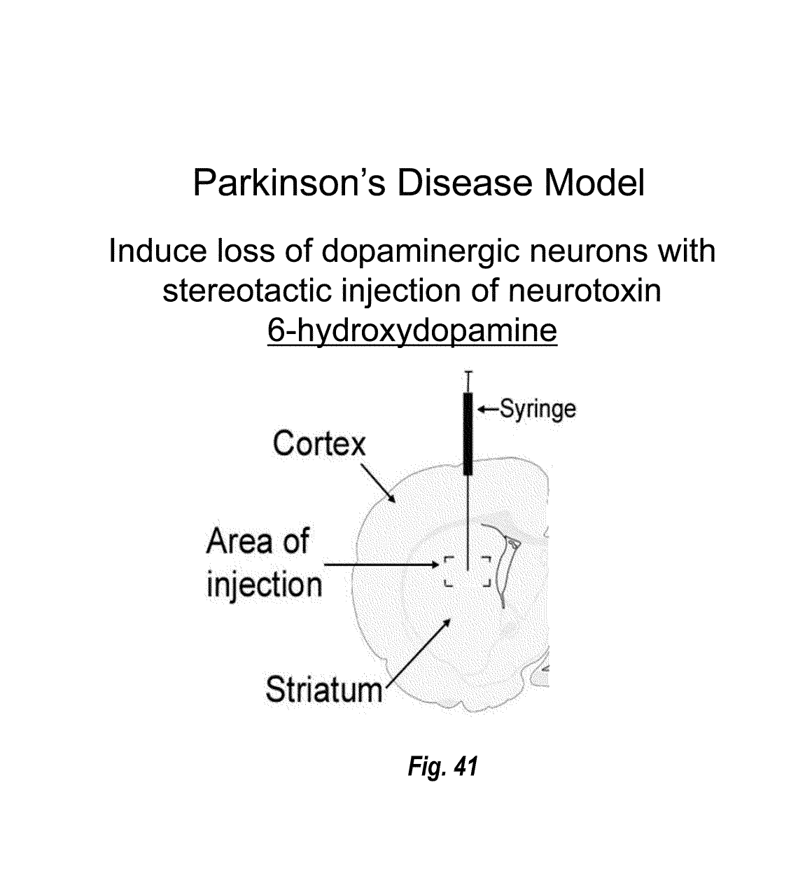

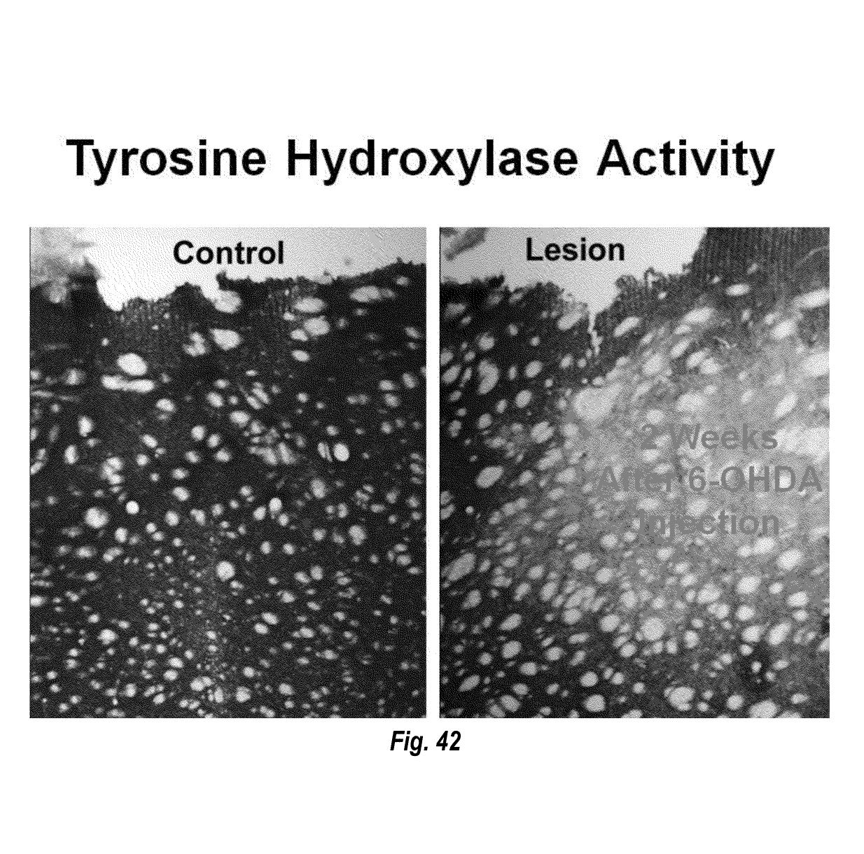

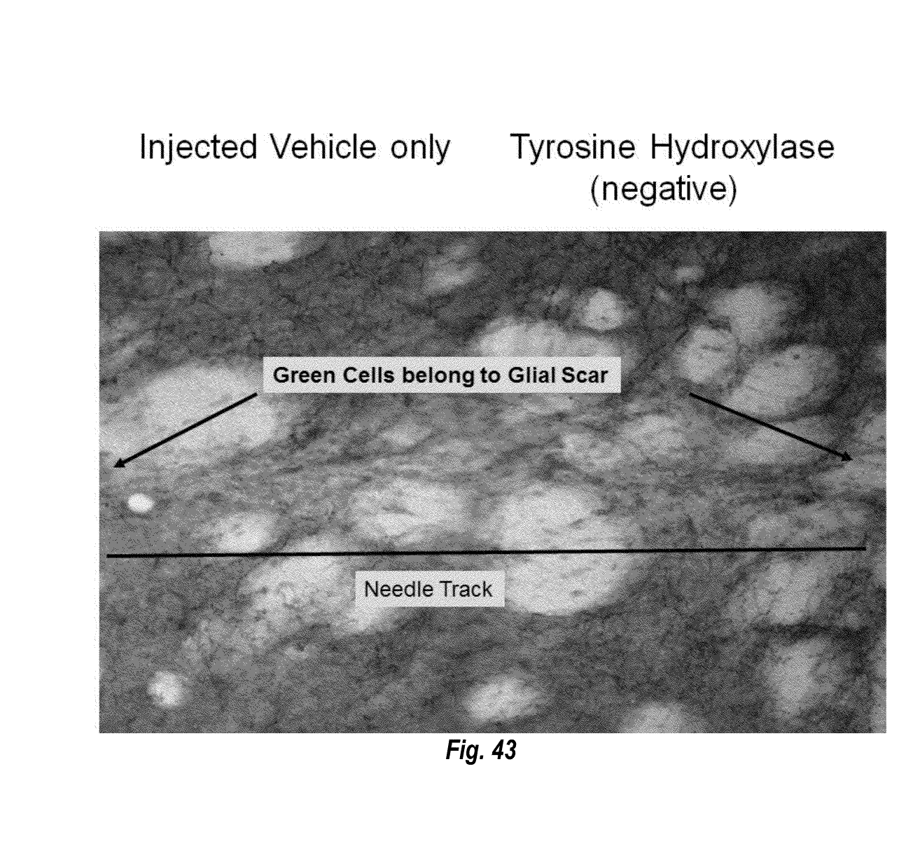

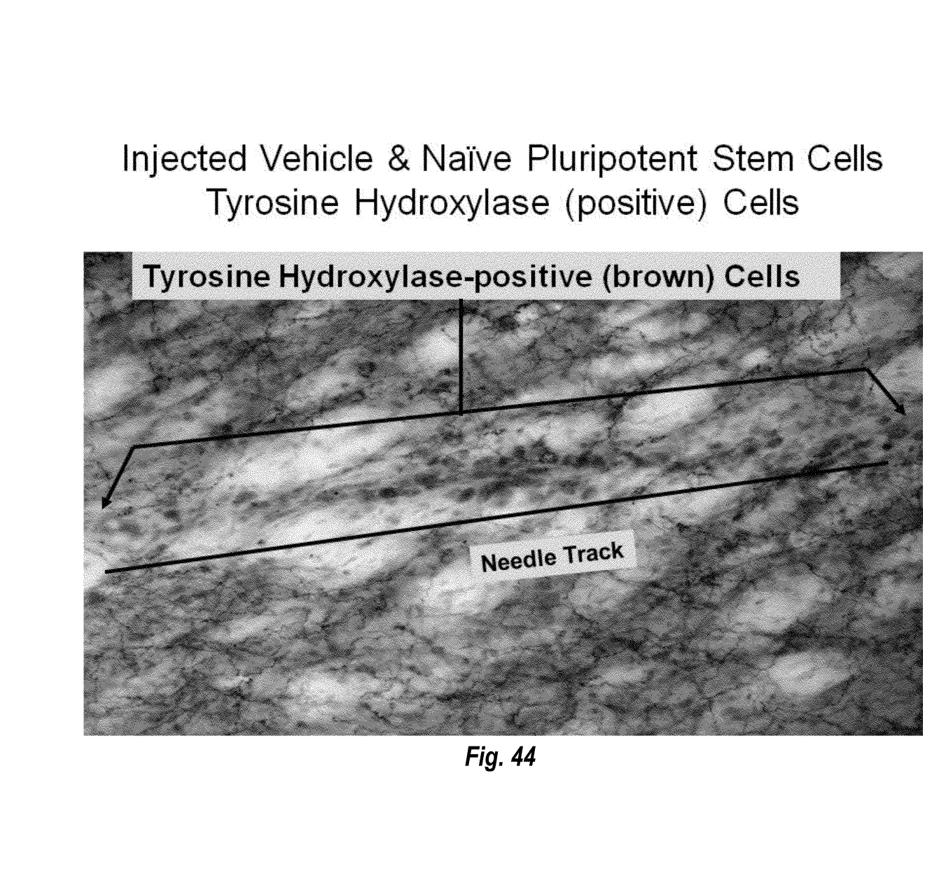

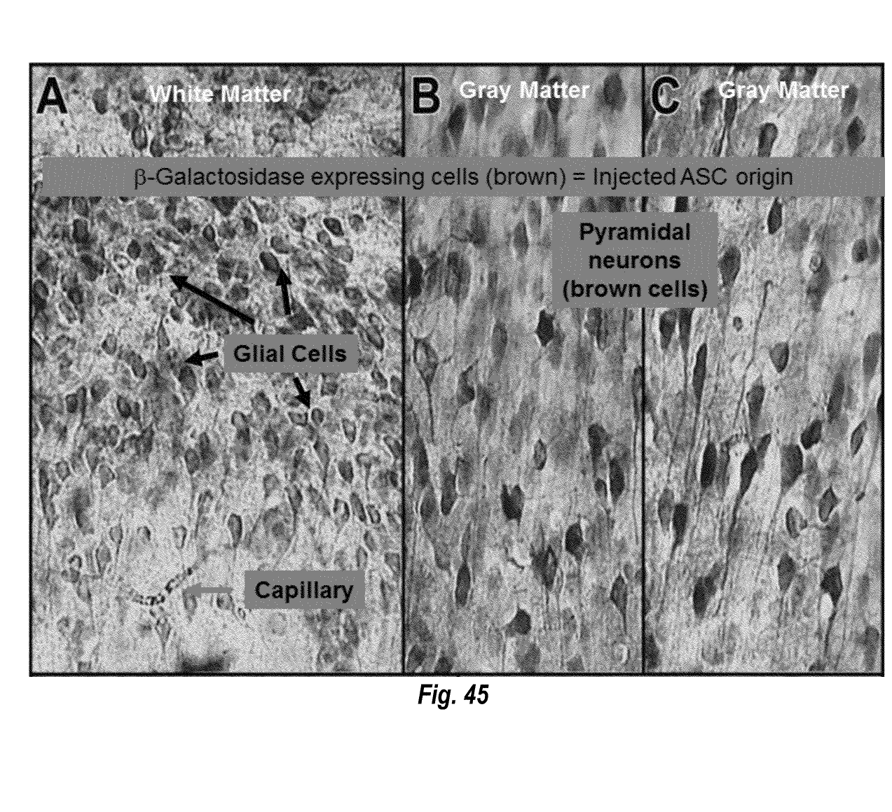

The Parkinson model examined was a 6-hydroxydopamine-induced substantia niagral-lesioned in the midbrain (FIG. 41). In this instance, genomically-labeled pluripotent stem cells were used. Two weeks after creating the lesion by stereotactic injection of the neurotoxin (FIG. 42), the vehicle buffer control or the vehicle plus the genomically-labeled pluripotent stem cells were stereotactically placed in the lesioned niagral area of the midbrain. Vehicle only (FIG. 43) demonstrated formation of glial scar tissue. Vehicle plus stem cells (FIG. 44) regenerated dopaminergic neurons at the lesion site. However, the genomically-labeled regenerative pluripotent stem cells also migrated away from the site of injection and repaired all the cells and tissues damaged during the repeated stereotactic injections (FIG. 45). These cells and tissues included pyramidal neurons, glial cells, and patent capillaries in the cerebral cortex (Young et al., 2005).

### Mesodermal Lineage Repair: Myocardial Infarction

We examined two myocardial infarction models in adult male rats. One myocardial infarction model was created by freezing the tip of the left ventricle with liquid nitrogen. The other myocardial infarction model was created by a transient ligation of the left anterior descending coronary artery (LAD).

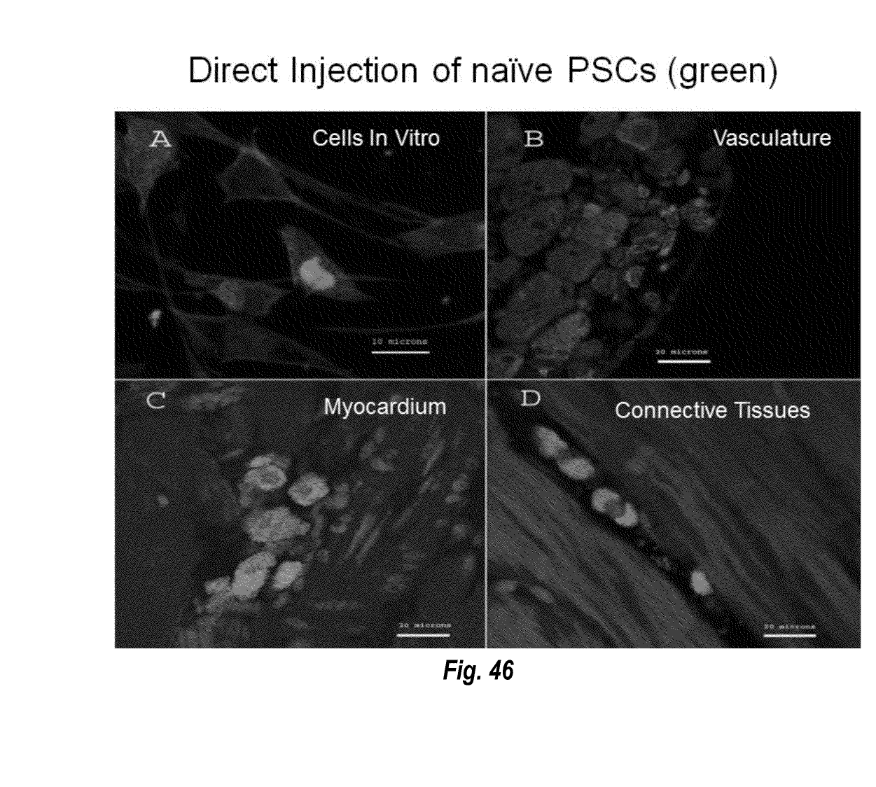

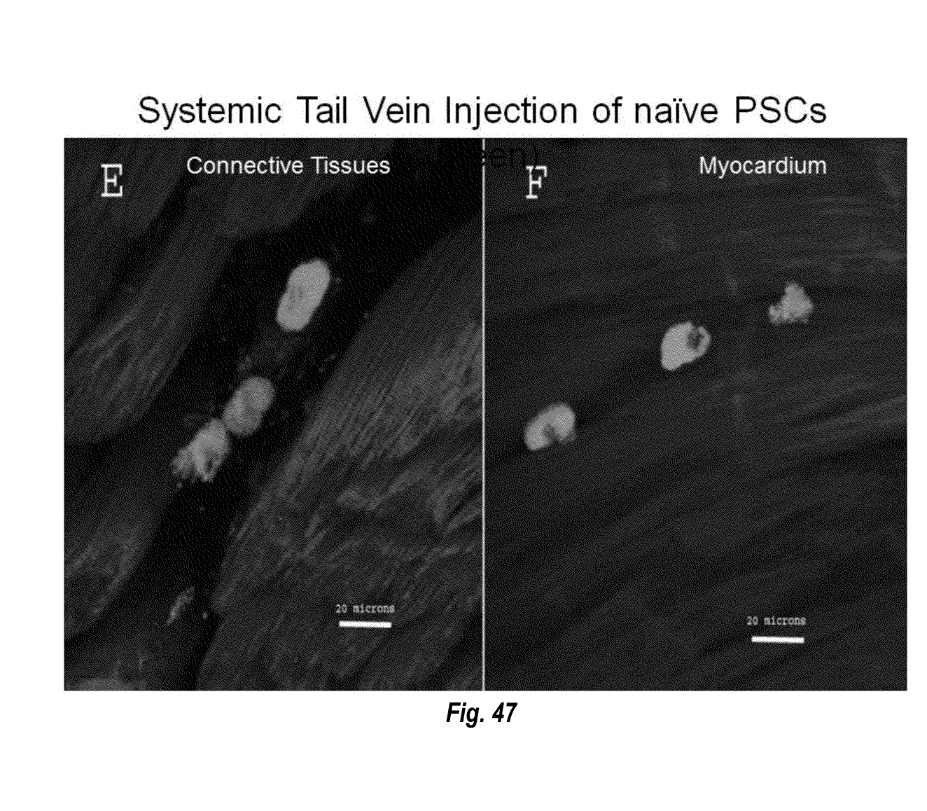

In the first model system, regenerative pluripotent stem cells (Scl-40β) were injected into the apex of the left ventricle, the animals allowed to recover from one to six weeks and then euthanized, the tissue removed, cryosectioned at 50 microns, stained with Rhodamine, and viewed with a con-focal microscope. After 4 weeks, β-galactosidase containing cells were located in the vasculature, in the healing myocardium, and in the surrounding perimysial connective tissues (FIG. 46). Next, the genomically-labeled stem cells were injected into the tail vein of rats, where their left ventricle apexes had been previously frozen. This variant of the model was used to examine whether the stem cells would migrate to damaged tissues. As shown, genomically labeled cells were detected in the connective tissue, the myocardium (FIG. 47) and the vasculature (data not shown). Other random tissues were selected for analysis and no genomically labeled cells could be found in those tissues (data not shown).

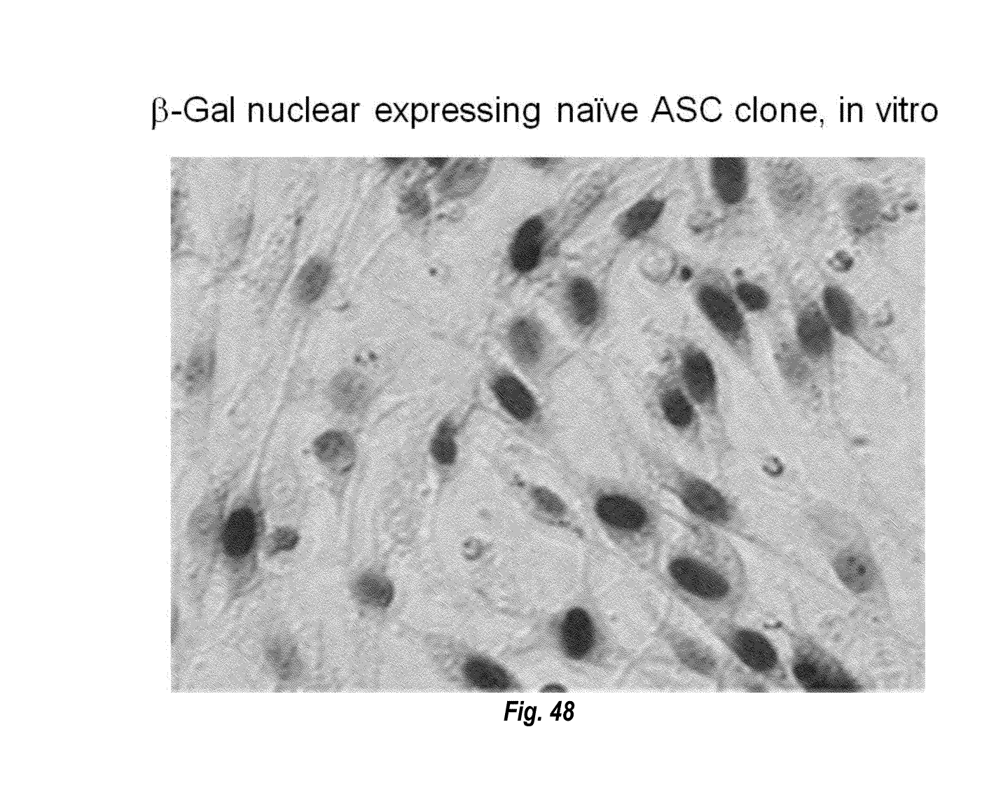

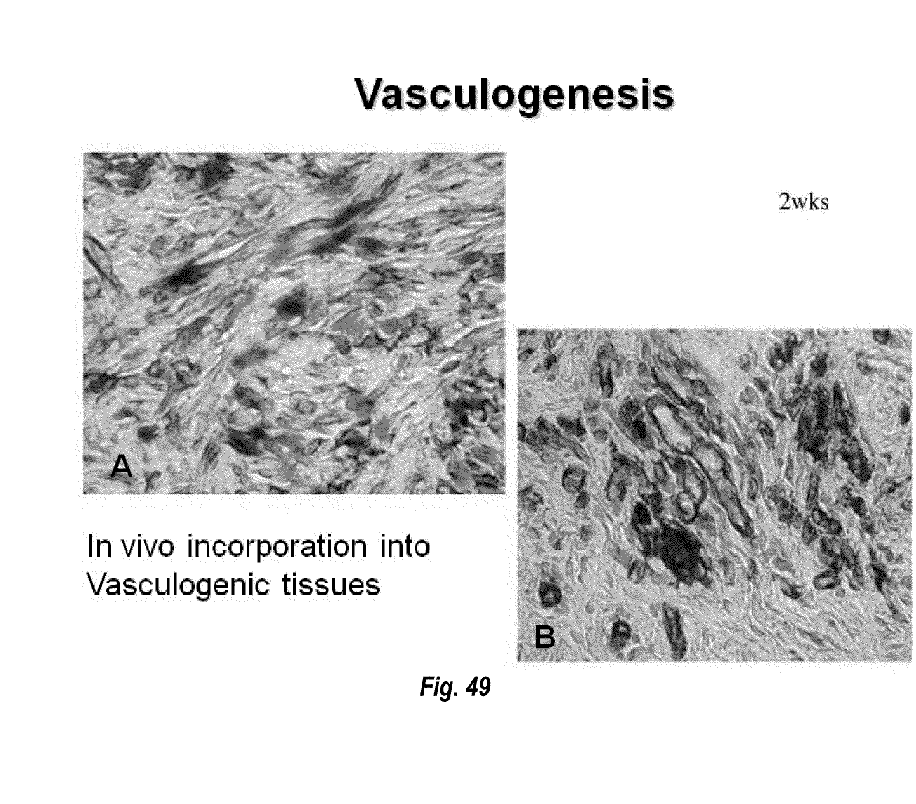

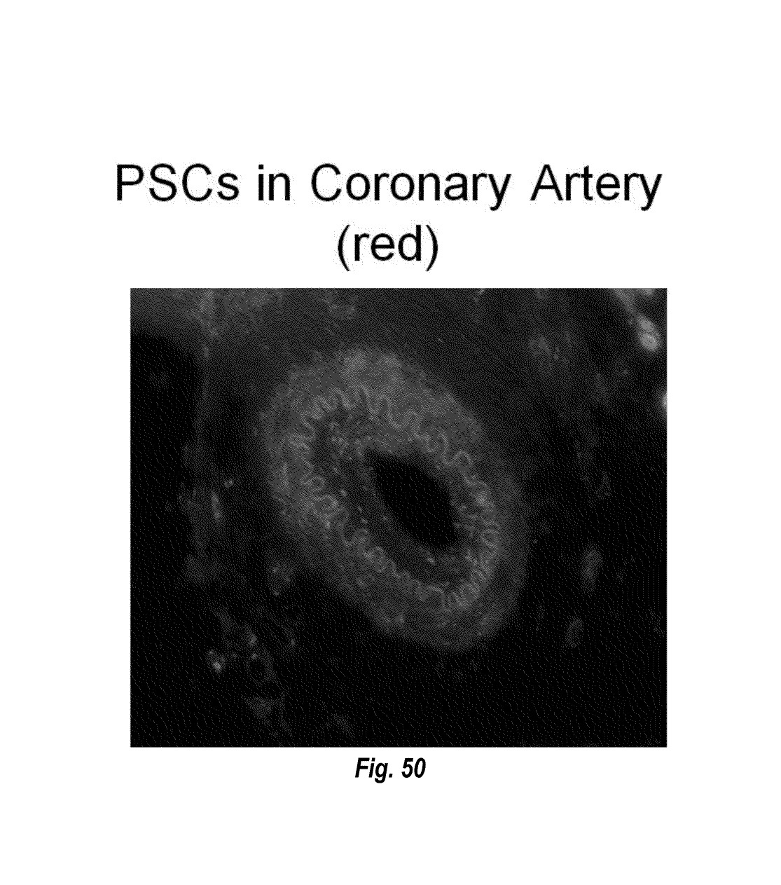

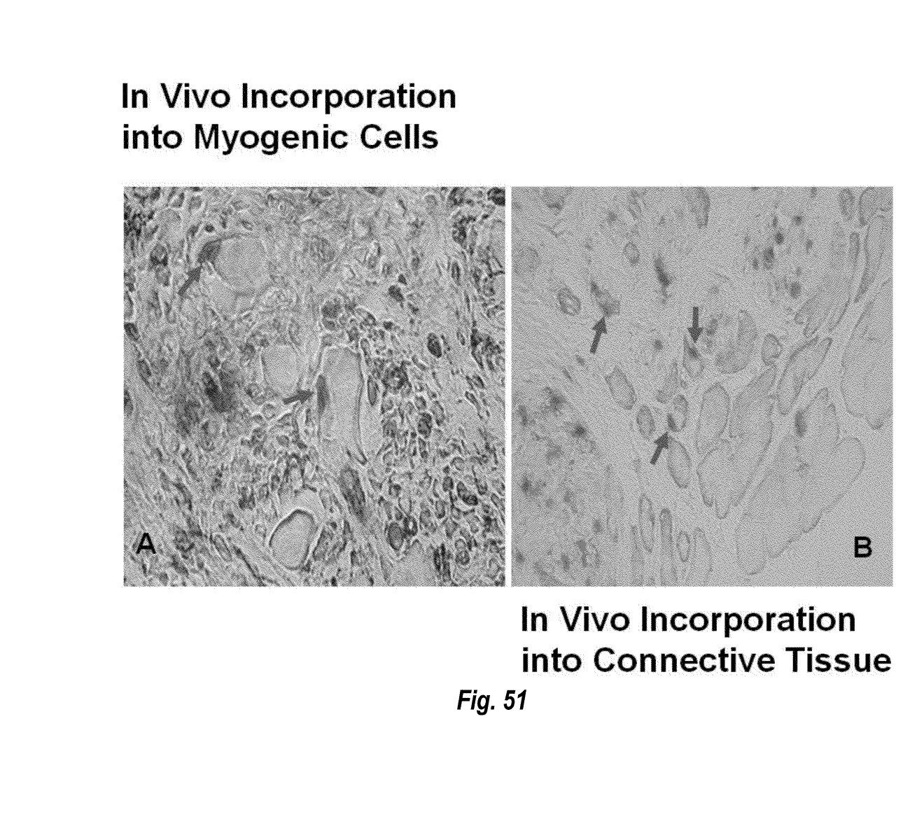

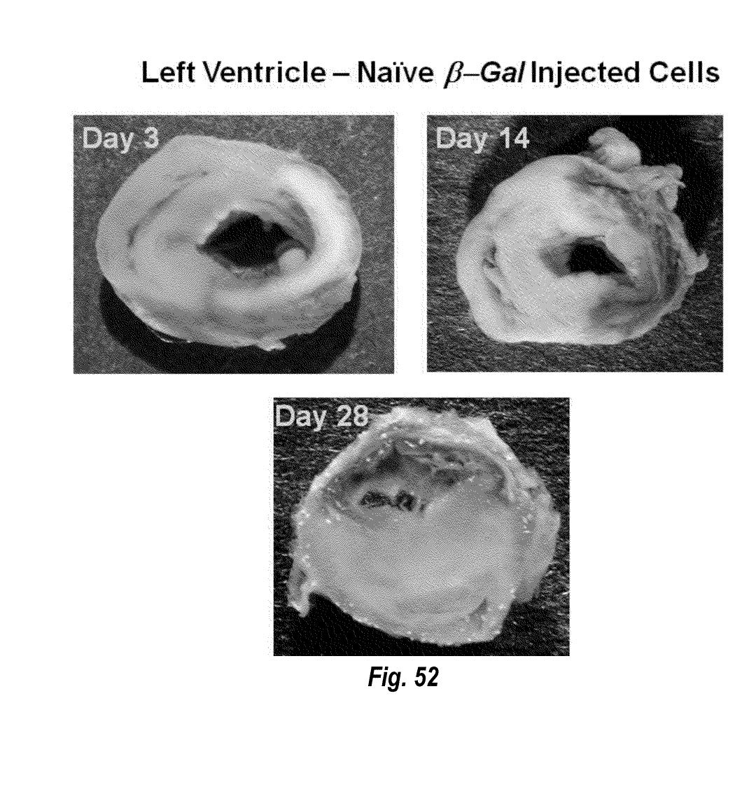

In the second model system, beta-galactosidase histochemistry (blue) of LacZ-genomically-labeled Scl-40β-ELSCs in vitro was shown. Note, there were blue-green stained nuclei, this was indicative that greater than 90% of cells retained genomic material within their respective nuclei after many cell doublings (FIG. 48). Beta-galactosidase histochemistry (blue) showed LacZ-genomically-labeled Sci-40β ELSCs into vasculogenic tissues of the heart two weeks after transient left anterior descending arterial ligation (FIG. 49). Fluorescence microscopy demonstrated incorporation of LacZ genomically-labeled pluripotent epiblast-like stem cells into the left anterior-descending coronary artery after transient ligation (FIG. 50). Note red-stained beta-galactosidase appearing cells in the tunica intima, tunica media, and tunica adventitia of the small muscular artery. Note also green wavy lines denoting internal and external elastic membranes (FIG. 50). Beta-galactosidase histochemistry (blue) of LacZ-genomically-labeled Scl-40β-ELSCs: A—into myocardium and B—into connective tissues of the heart two weeks after transient left anterior descending arterial ligation (FIG. 51). Beta-galactosidase histochemistry (blue) of LacZ-genomically-labeled Scl-40β-ELSCs remained viable in the myocardium of left ventricle of the heart at Day 3, Day 14, and Day 28 (termination of experiment), after transient left anterior descending arterial ligation (FIG. 52).

### Endodermal Lineage Repair: Artificial Pancreas

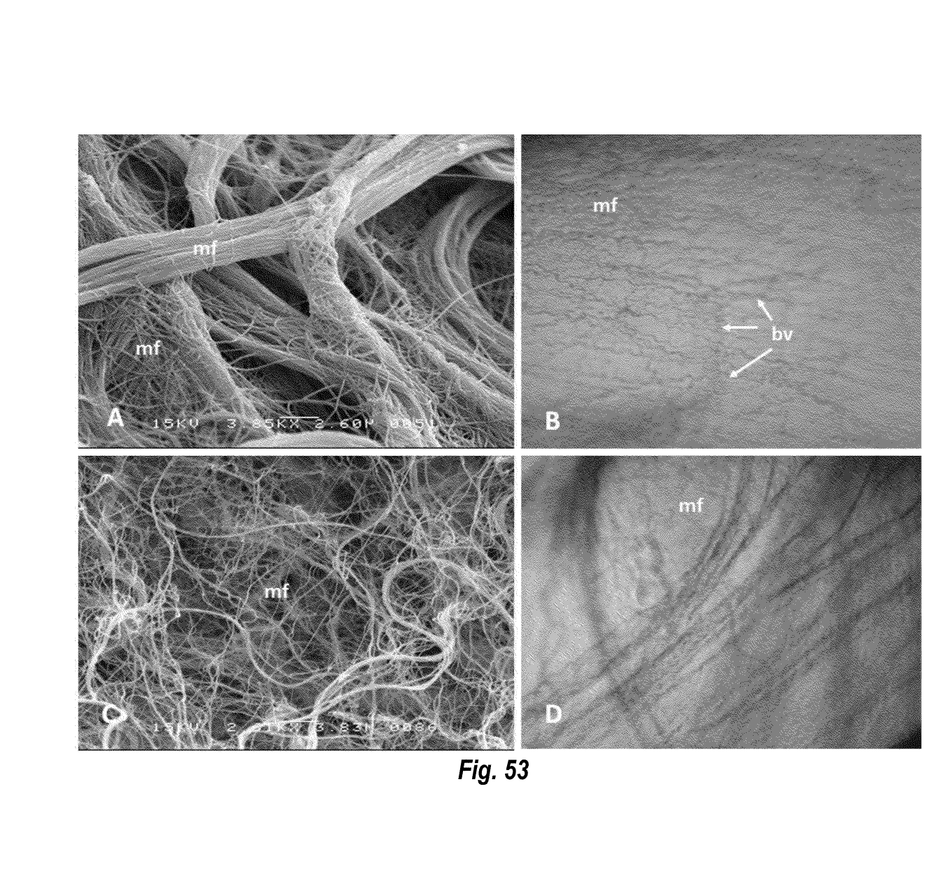

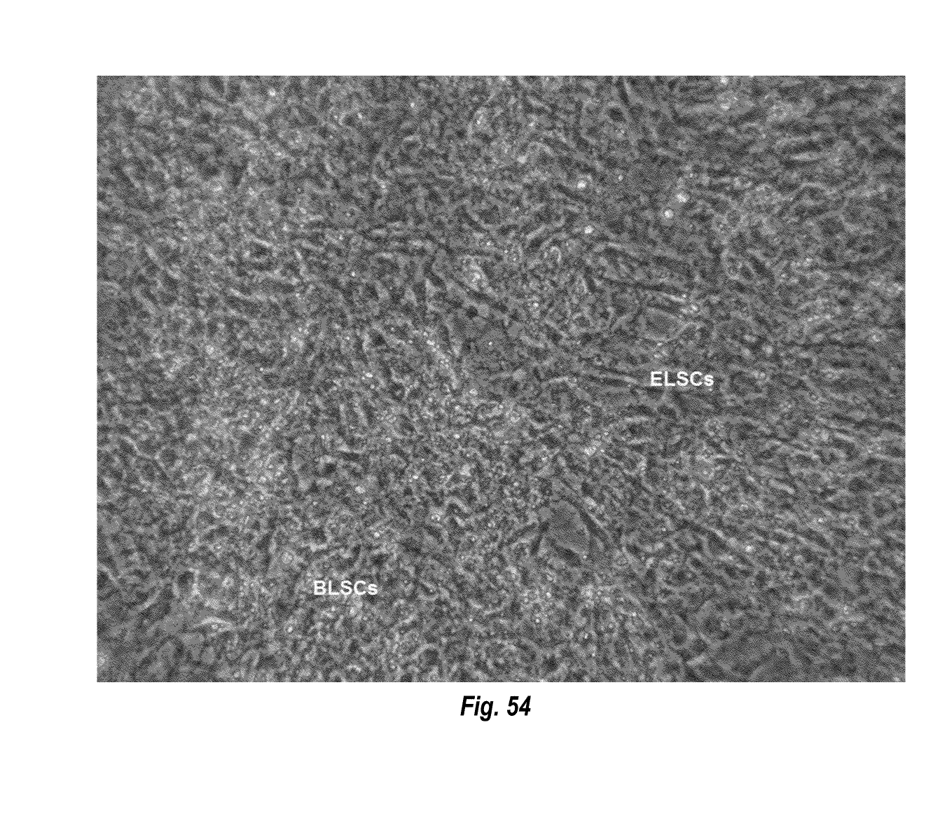

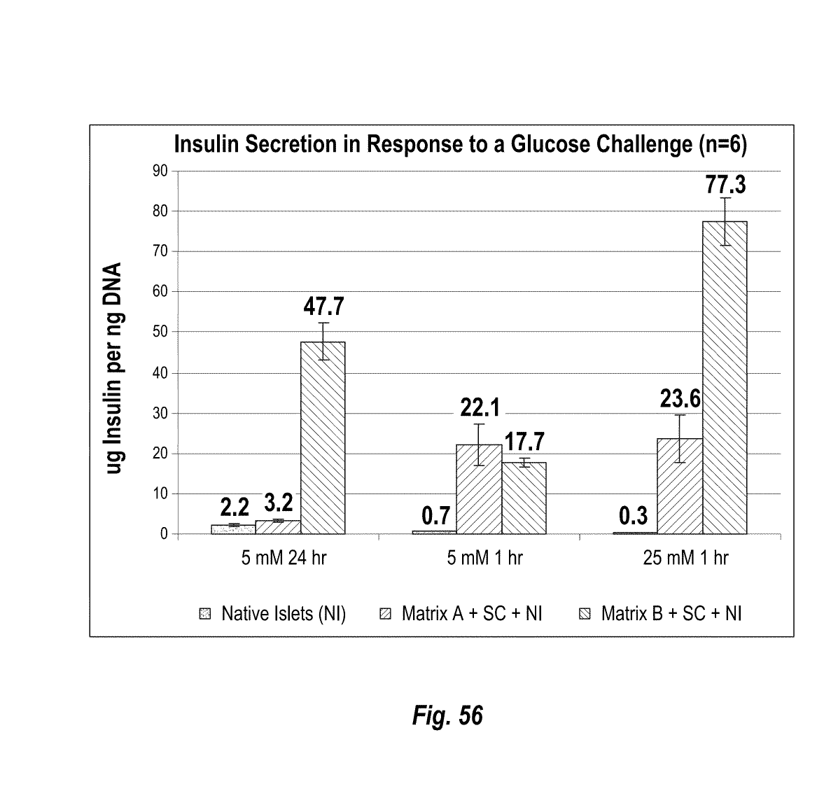

An artificial pancreatic organoid was produced using a combinatorial approach. This approach consisted of using decellularized porcine pancreatic matrices (FIG. 53), a mixture of adult rat regenerative pluripotent stem cells (FIG. 54) and native rat pancreatic islets (55). When examined using a radioimmunoassay specifically directed against rat insulin, the combinatorial islet-like organoids secreted 78.6 (matrix-A) and 257.6 (matrix-B) times the amount of insulin per nanogram DNA as native islets alone (FIG. 56). This occurred after being challenged sequentially with first 5 mM glucose and then with 25 mM glucose as the secretagogue inducing agent.

### SUMMARY OF THE INVENTION

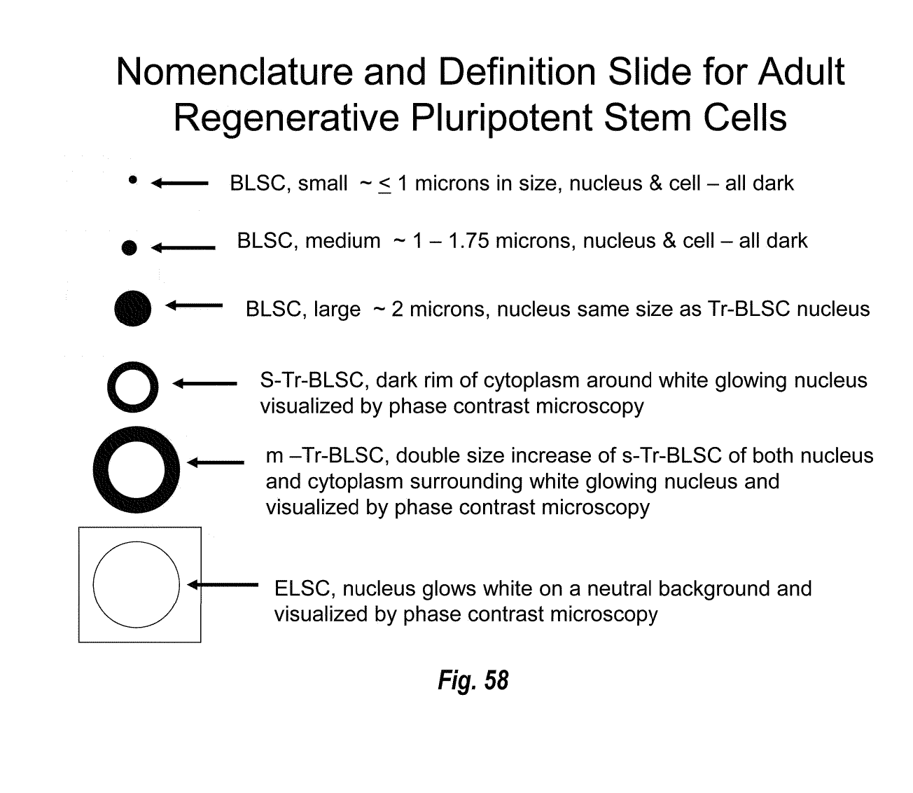

The present invention is directed to compositions and method related to adult-derived regenerative pluripotent transitional-blastomere-like stem cells that express telomerase and carry surface markers CD10⁺, SSEA⁺ and a halo of CD66e⁺, CEA-CAM-1⁺, and Trypan blue staining (FIGS. 58,59).

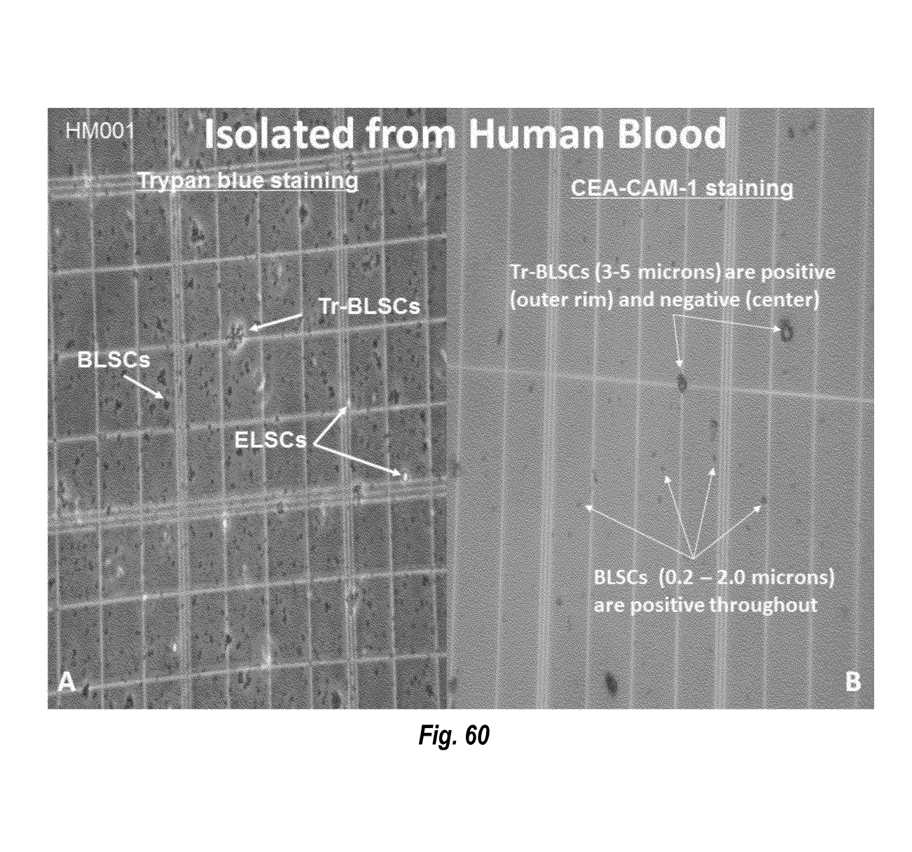

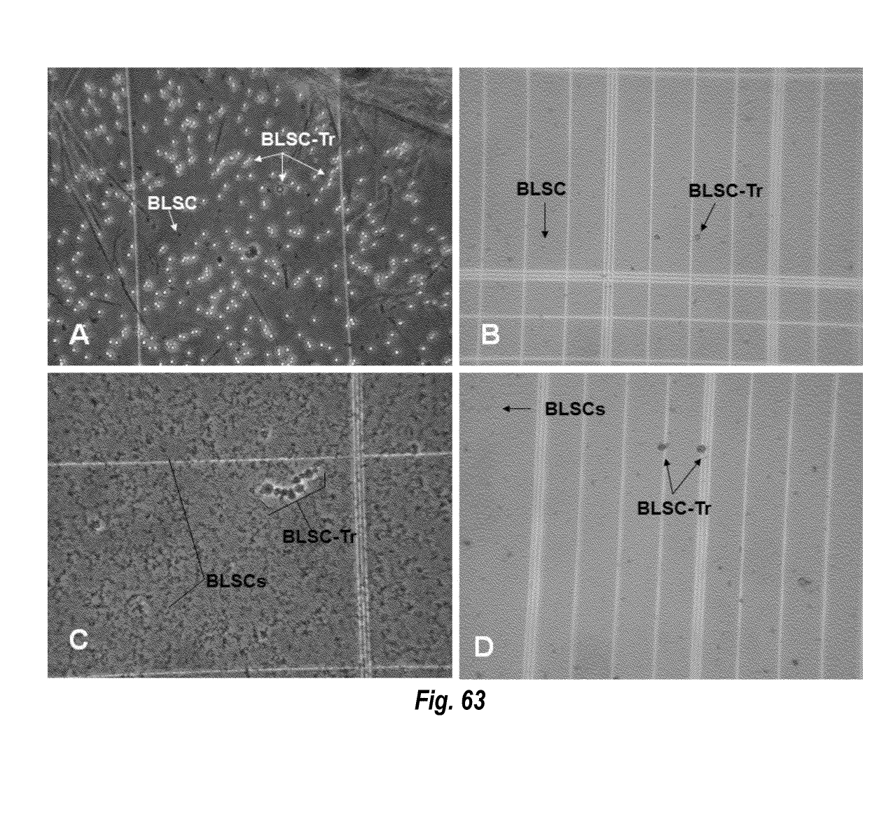

In one aspect of the inventive subject matter, an isolated stem cell is preferably a human post-natal, pluripotent stem cell having surface markers CD10⁺, SSEA⁺ and a halo of staining of CD66e⁺, CEA-CAM-1⁺, and Trypan blue (FIGS. 60-62) that are 3 to 5 microns in size, as assessed by flow cytometry of living cells (FIG. 7) (Table 1). In another aspect of the inventive subject matter, an isolated stem cell is preferably a non-human mammalian post-natal, regenerative pluripotent stem cell having surface markers SSEA⁺ and a halo of staining of CEA-CAM-1⁺ and Trypan blue (FIGS. 63-67) that are 3-5 microns in size as assessed by flow cytometry of living cells (Table 1).

Such cells advantageously differentiate into an epiblast-like stem cell upon stimulation with a differentiating medium, and are known to undergo at least 100 population doublings while maintaining undifferentiated pluripotent characteristics in serum-free medium in the absence of differentiation inhibitors. Thus, these stem cells, according to the inventive subject matter, will typically not spontaneously differentiate in defined serum-free medium in the absence of differentiation inhibitors. Rather, they will remain quiescent and will not form a cancerous tissue when implanted into an animal or human (Table 1).

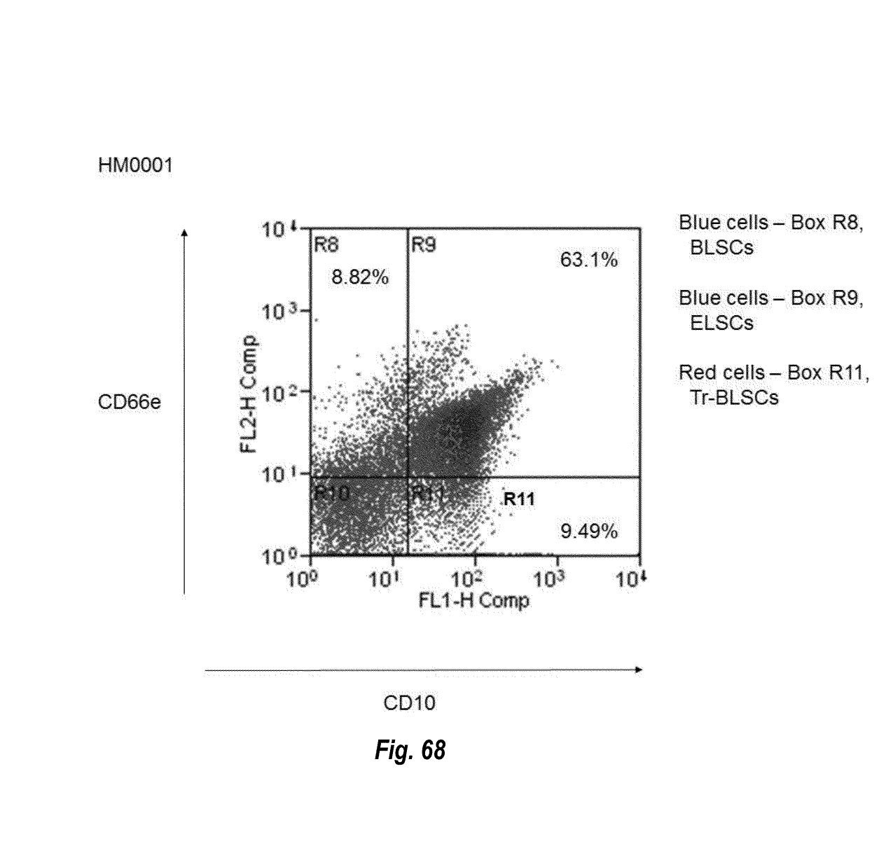

Such human cells may further be characterized by expression of telomerase, Oct-3/4, Sonic hedgehog, CD66e/CD10 joined cell surface markers (FIG. 68) and lack of expression of BMI-1, IDE1, IDE3, ABCG2, CXCR-4, BCL-2, CD1a, CD2, CD3, CD4, CD5, CD7, CDB, CD9, CD11b, CD11c, CD13, CD14, CD15, CD16, CD18, CD19, CD20, CD22, CD23, CD24, CD25, CD31, CD33, CD34, CD36, CD38, CD41, CD42b, CD45, CD49d, CD55, CD56, CD57, CD59, CD61, CD62E, CD65, CD68, CD69, CD71, CD79, CD83, CD90, CD95, CD105, CD106, CD117, CD123, CD135, CD166, Glycophorin-A, MHC-I, HLA-DRII, FMC-7, Annexin-V, and/or LIN.

### Adult Tissue Location of Regenerative Pluripotent Transitional-Blastomere-Like Stem Cells

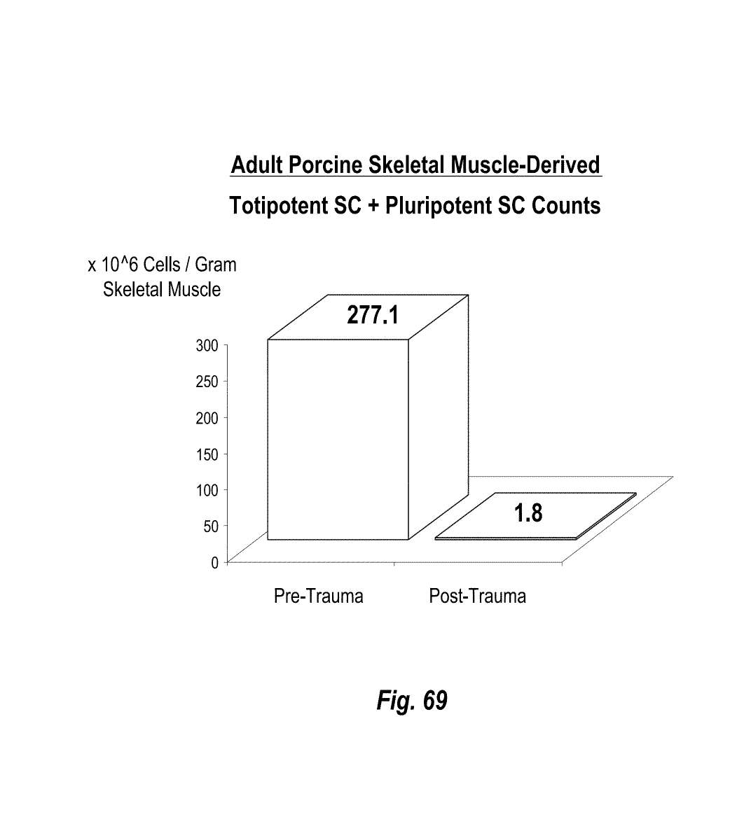

Previous studies have shown that adult regenerative pluripotent stem cells, i.e., totipotent blastomeric-like stem cells, epiblast-like stem cells, and germ layer lineage stem cells, reside within niches within the connective tissue matrices of the adult differentiated tissues (FIGS. 18-29). Where upon activation, the regenerative pluripotent regenerative stem cells divide at a rate similar to BLSCs and ELSCs (i.e., once every 12-14 hours) (Young et al., 2004a; Young and Black, 2012) and the extra daughter stem cells migrate into the vasculature to travel to the sites of injury and repair the injured tissue (FIGS. 69,70) [Stout et al., 2007].

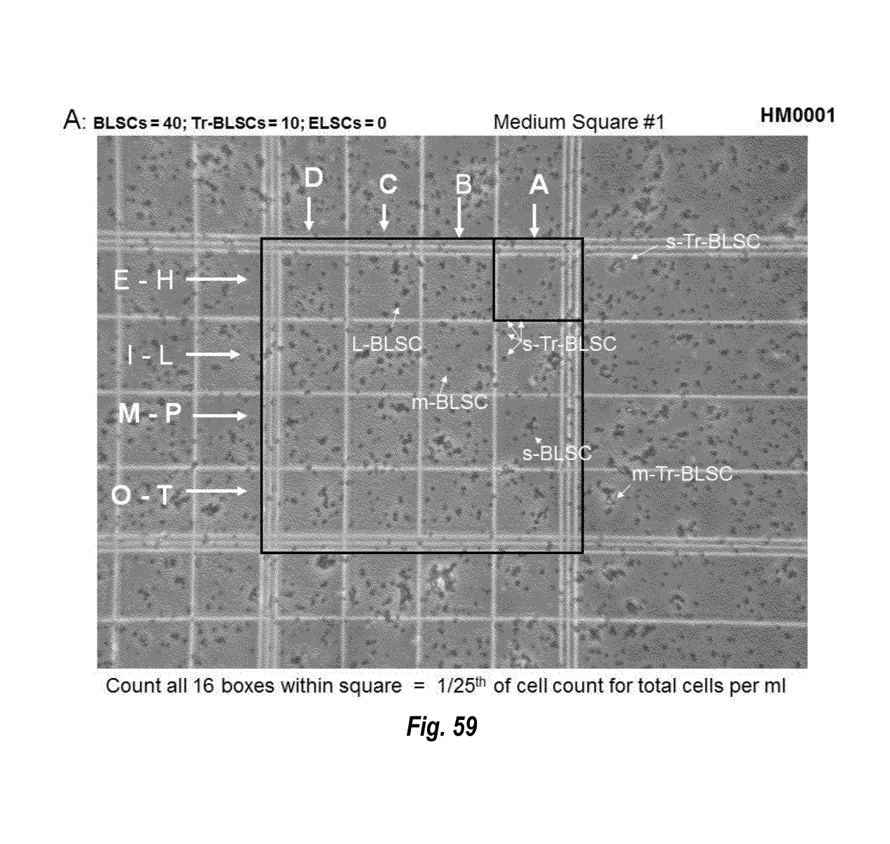

As mentioned above, transitional pluripotent blastomere-like stem cells, i.e., those stem cells that occur between the previously defined stem cell types, also are located in the tissues and progress throughout the differentiation pathway. In this case, the stem cell traverses between blastomeric-like stem cells and epiblast-like stem cells. Although the regenerative pluripotent “transitional blastomeric-like stem cells” (Tr-BLSCs) share similar cell surface markers as both blastomere-like stem cells (BLSCs) and epiblast-like stem cells (ELSCs), their size differences and staining peculiarities make them recognizable as a separate regenerative stem cell type (FIGS. 59-65), which can be isolated and used in place of blastomere-like stem cells (BLSCs) or epiblast-like stem cells (ELSCs) to repair somatic tissues (Table 1).

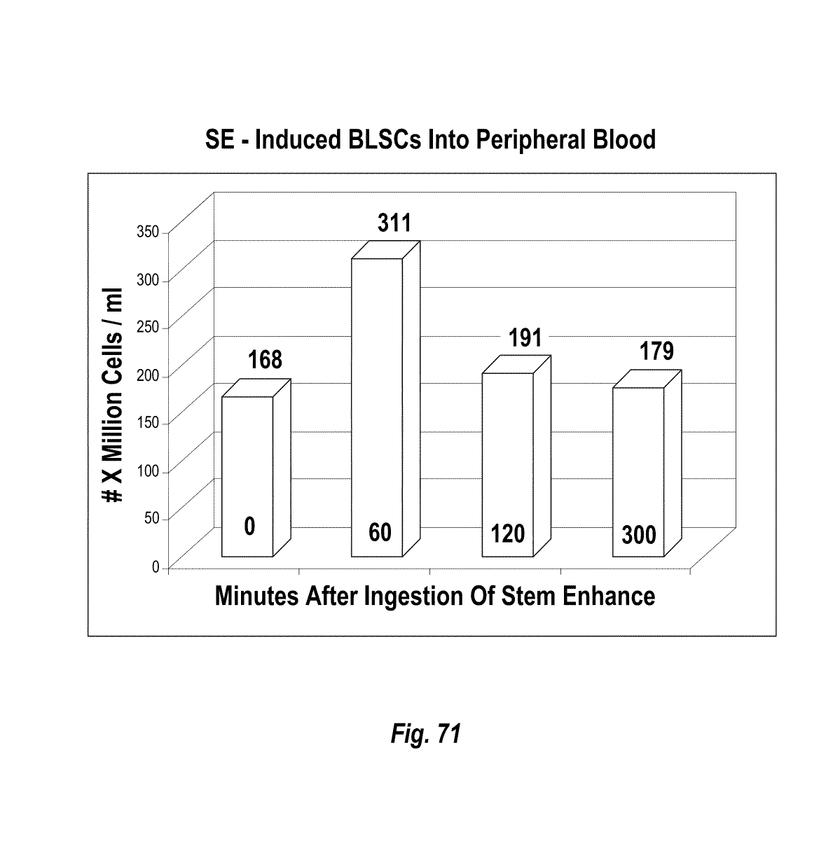

In still further contemplated aspects, the inventor contemplates a method of multiplying the adult-derived regenerative pluripotent blastomere-like stem cells in situ (in vivo). This was first attempted using Stem Enhance (FIG. 71), a nutraceutical advertised on the internet, that is suppose to increase the number of adult stem cells in the blood. When taken at the recommended dosage of 8 capsules per day the number of stem cells in the blood at 60 min approximated 1500% of normal for 20 human males (e.g., n=20, HM0011 to HM0021). When taken long term, this number (˜1500% or ˜4.5 billion cells per ml) held its value for two weeks, then plummeted to approximately 1% (˜1.68 million cells per 1) of normal value and continued at that value as long as the Stem Enhance was taken. During the first two weeks, with high amounts of stem cells circulating in the blood, healing from trauma occurred very quickly. After 14 weeks, healing from trauma was next to non-existent (anecdotal comments from HM0011 to HM0021). These results suggest that high dose of Stem Enhance for short period of time (e.g., 14 weeks) depleted the supply of mother stem cells as well as the transient daughter stem cells within the tissue connective tissue niches leaving very few mother stem cells behind to propagate and release stem cells for repair.

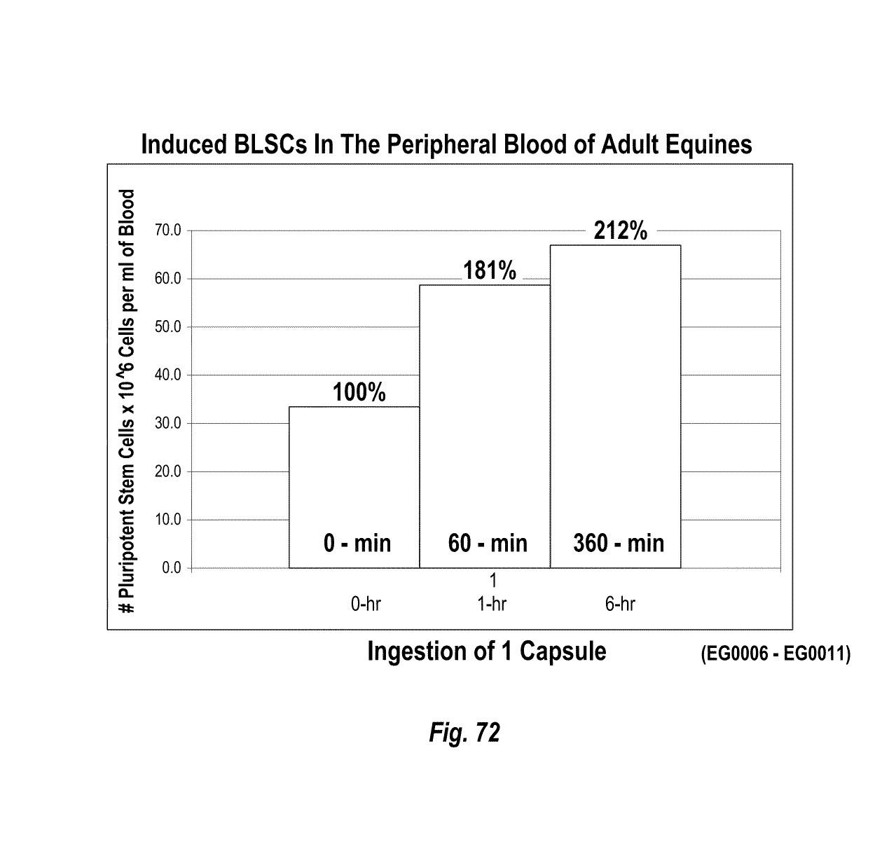

We have since discovered a “compound” that, when taken properly, slowly increases the number of daughter stem cells in the blood without depleting the body of its resident supply of mother stem cells (FIG. 72). This compound is composed of a mixture of the following plant-based materials and placed into 500 milligram capsules. The 500 milligrams of material contains and 500-2500 kCal, i.e., protein 50-100%, fat 0-50%, minerals 1-20%, lipids 1-10%, pigments 1-10%, moisture 1-10%, chlorophyll 50-100%, alpha-linolenic acid (Omega-3) 10-50 mg, gamma-linolenic acid 1-30 mg, provitamin-A beta carotene 400-5000 IU, thiamine (B1) 1-100 mcg, riboflavin (B2) 1-100 mcg, niacin (B3) 1-100 mcg, pantothenic acid (B5) 1-100 mcg, pyridoxine (B6) 1-100 mcg, inositol 1-100 mcg, vitamin D 1-100 mcg, vitamin E 1-100 IU, ascorbic acid (vitamin C) 1-100 mcg, biotin 1-1000 mcg, folic acid 1-1000 mcg, choline 1-1000 mcg, cobalamin (B12) 1-1000 mcg, vitamin K 1-1000 mcg, boron 1-1000 mcg, calcium 1-1000 mcg, chloride 1-1000 mcg, chromium 1-1000 mcg, cobalt 1-1000 mcg, copper 1-1000 mcg, fluoride 1-1000 mcg, germanium 1-1000 mcg, iodine 1-1000 mcg, iron 1-1000 mcg, magnesium 1-1000 mcg, molybdenum 1-1000 mcg, nickel 1-1000 mcg, potassium 1-1000 mcg, phosphorous 1-1000 mcg, selenium 1-1000 mcg, silicon 1-1000 mcg, sodium 1-1000 mcg, tin 1-1000 mcg, titanium 1-1000 mcg, vanadium 1-1000 mcg, and zinc 1-1000 mcg. The compound is given at increasing capsules following a defined schedule. With increasing defined intervals of time the compound increases the number of adult-derived regenerative pluripotent blastomere-like stem cells circulating in the peripheral vasculature.

### Adult Tissue Isolation of Regenerative Pluripotent Transitional-Blastomere-Like Stem Cells by Multiple Techniques

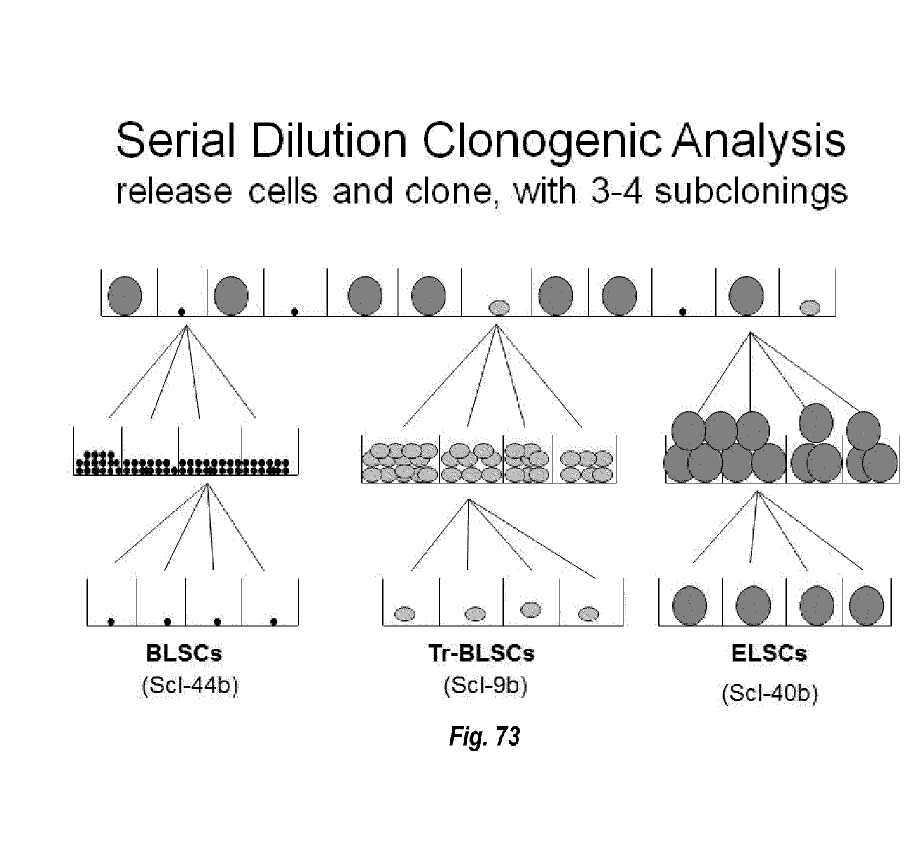

In still further contemplated aspects, the inventor contemplates a method of harvesting the adult-derived regenerative pluripotent transitional blastomere-like stem cells from their in situ (in vivo) location in the blood. Once the individual takes the appropriate number of compound capsules to increase the number of adult-derived regenerative pluripotent transitional blastomere-like stem cells within the blood, 1.0-ml to 4000.0-ml of blood is removed by sterile venipuncture. The blood is placed into sterile tubes or an IV bag containing EDTA and then placed into a 4 degree centigrade/38 degree Fahrenheit refrigerator for 20 minutes to 96 hours, dependent on the species. Multiple techniques can then be utilized to separate the stem cells, i.e., BLSCs, Tr-BLSCs, ELSCs, and GLSCs from the hematopoietic elements (FIGS. 73-77).

Due to the inherent nature of the zeta-potential on the surface of the hematopoietic red blood cells, hematopoietic white blood cells and the adult-derived regenerative pluripotent stem cells, the hematopoietic cells and stem cells self-separate from each other by quantitative polyanionic zeta potential repulsion. The hematopoietic cells precipitate to the bottom of the tube, as the red cell layer with the white cell buffy coat layer on top. The adult-derived regenerative pluripotent stem cells are maintained in the serum layer by the quantitative polyanionic zeta potential repulsion. Depending on the particular species, i.e., mouse, rat, rabbit, cat, dog, sheep, goat, pig, cow, horse, or human, the minimal time for separation of the stem cells can be as few as 20 minutes or as long as 96 hours. The serum layers, containing the adult-derived regenerative pluripotent regenerative pluripotent stem cells, i.e., blastomeric-like stem cells, transitional blastomere-like stem cells, and epiblast-like stem cells, are removed in toto, washed with an equal volume of sterile saline, and centrifuged at high speed for 5 minutes to 60 minutes. The supernatant is removed to sterile saline bags for intravenous (IV) infusion into the individual. The pelleted stem cells are resuspended, pooled, and a small 200 microliter sample is removed for counting. The counted adult-derived regenerative pluripotent stem cells are then used at appropriate quantities to augment tissue repair, where needed. The remaining layers of serum, containing the germ layer lineage stem cells (next to the buffy coat) are removed, pooled, and placed into the sterile saline bags for infusion into the individual. Adult-derived regenerative pluripotent transitional blastomere-like stem cells may be stored up to 30 days from 1-30 degrees centigrade with minimal loss of viability.

### Transplantation of Adult Regenerative Pluripotent Transitional-Blastomere-Like Stem Cells

In still further contemplated aspects, the inventor contemplates a method of using the adult-derived regenerative pluripotent blastomere-like stem cells for transplantation therapies. Such therapies might include, but are not exclusive to, neurological diseases (i.e., Parkinson's disease, Alzheimer's disease, Dementia, Stroke, Neuropathies, Neuroparesthesias, Sciatica, Multiple Sclerosis, Amyotrophic Lateral Sclerosis, Spinal Cord Injury, etc.), cardiovascular diseases (myocardial infarction, cardiac hypertrophy, cardiac ischemia, vascular ischemia, etc.), pulmonary diseases (COPD, IPF, bronchitis, emphysema, asthma, cystic fibrosis, etc.), gastrointestinal diseases (i.e., Celiac disease, Crohn's disease, etc.), urinary diseases (i.e., polycystic kidney disease, urinary bladder obstructions, valvular weakness of the urinary sphincter, etc.); autoimmune diseases (i.e., Hashimoto's thyroiditis, type-I diabetes, scleroderma, lupus, systemic lupus erthythematosus, rheumatoid arthritis, etc.); orthopedic disorders (i.e., bone fractures, bone loss, osteo-arthritis, articular joint destruction, Lyme's disease, rotator cuff tears, articular joint sprain, skeletal muscle tears, ligament and tendon evulsions or tears, cartilage (i.e., elastic, hyaline, growth plate, articular, and fibrocartilage) replacement or repair; burn injuries (preferably third degree burns, but also second and first degree burns); transection of the skin and underlying soft tissues due to trauma or elective surgeries; etc. Typically, implantation of the adult regenerative transitional blastomeric-like stem cell will be into damaged tissues, after scar tissue has been removed, in an elective plastic surgery-type setting, and/or when the tissue is undergoing repair.

### Human Models of Tissue Replacement and Repair

### Human Parkinson Study with Compound and Stem Cells

The overall objective is to mobilize autologous adult regenerative pluripotent stem cells into the peripheral blood stream in situ, at sufficient quantities to provide a source of autologous adult stem cells for cell, tissue, and organ-associated Parkinson's disease repair. We will use a Parkinson's disease (PD) population. We will assess the core aspects of PD by targeting specific outcomes related to function, cognition, affect and caregiver burden. We will then remove 500-ml of blood after 3 months on ingestion of the “compound” to isolate regenerative pluripotent stem cells. We will do this on three occasions for a year (3 capsules per day, 6 capsules per day, and 8 capsules per day). These autologous stem cells will be isolated from the blood, one-half of the isolated pluripotent stem cells will be infused into the patient's central nervous system via an intra-nasal route, and the remaining half of the isolated pluripotent stem cells will be given back to the peripheral vasculature of the patient via a 500-ml normal saline IV-drip. We will then assess the overall improvement of the cognitive aspects of PD by targeting specific outcomes related to function, cognition, affect and caregiver burden at one month, 3 months, and six months post infusion.

The compound is composed of the following constituents in 0.500 g capsules and containing 500-2500 kCal, i.e., protein 50-100%, fat 0-50%, minerals 1-20%, lipids 1-10%, pigments 1-10%, moisture 1-10%, chlorophyll 50-100%, alpha-linolenic acid (Omega-3) 10-50 mg, gamma-linolenic acid 1-30 mg, provitamin-A beta carotene 400-5000 IU, thiamine (B1) 1-100 mcg, riboflavin (B2) 1-100 mcg, niacin (B3) 1-100 mcg, pantothenic acid (B5) 1-100 mcg, pyridoxine (B6) 1-100 mcg, inositol 1-100 mcg, vitamin D 1-100 mcg, vitamin E 1-100 IU, ascorbic acid (vitamin C) 1-100 mcg, biotin 1-1000 mcg, folic acid 1-1000 mcg, choline 1-1000 mcg, cobalamin (B12) 1-1000 mcg, vitamin K 1-1000 mcg, boron 1-1000 mcg, calcium 1-1000 mcg, chloride 1-1000 mcg, chromium 1-1000 mcg, cobalt 1-1000 mcg, copper 1-1000 mcg, fluoride 1-1000 mcg, germanium 1-1000 mcg, iodine 1-1000 mcg, iron 1-1000 mcg, magnesium 1-1000 mcg, molybdenum 1-1000 mcg, nickel 1-1000 mcg, potassium 1-1000 mcg, phosphorous 1-1000 mcg, selenium 1-1000 mcg, silicon 1-1000 mcg, sodium 1-1000 mcg, tin 1-1000 mcg, titanium 1-1000 mcg, vanadium 1-1000 mcg, and zinc 1-1000 mcg. The compound is given at increasing capsules following a defined schedule. With increasing defined intervals of time the compound increases the number of adult-derived regenerative pluripotent blastomere-like stem cells circulating in the peripheral vasculature.

We utilized this compound, in a titratable fashion, to convert each participant into their own sterile bioreactor, to provide the requisite number of autologous regenerative pluripotent stem cells to affect a change in their cognitive status. We want to proliferate and then mobilize the connective tissue-resident pluripotent stem cells into the peripheral vasculature in an increasing, titratable, dose-response manner. To facilitate this process, we will apply a compound that mobilizes skeletal muscle connective tissue-resident adult pluripotent stem cells into the peripheral vasculature in an increasing dose-response manner. The compound has been shown to do this in horses (FIG. 59). The compound was started at a dosage of one capsule per day, in the morning, 30 minutes before breakfast, with water, on an empty stomach. The dosage was increased at monthly intervals, i.e., 2 capsules per day during the second month, and three capsules per day during the third month and throughout the remainder of the study.

To date, a collaborator has had over 50 people involved in a 36+ month study with the compound. Most of these people have been diagnosed with COPD (chronic obstructive pulmonary disease) or related lung illness, i.e., Interstitial Pulmonary Fibrosis, Emphysema, Bronchitis, etc. However, there are other participants in this trial that have non-COPD diagnoses, i.e., SLE (systemic lupus erythematosus), Epilepsy, Rotator Cuff Injuries, Cerebral Palsy, Musculoskeletal Diseases, Open Fractures, Osteoarthritis, Lyme's Disease, Cardiac Myopathies, Hypotonia, ALS (amyotrophic lateral sclerosis), Blindness, Spinal Cord Injury, Diabetes, and Parkinson's Disease. There have been no adverse effects of the compound reported on any of these subjects. Indeed, I received two letters from the manufacturer indicating that since 1979 and 1997, respectively, there have been no reported deaths from using their product. In the letter, referencing “1979” they state that every batch is run through extensive testing for microcysten, pesticides, heavy metals, mold, and yeast in five independent laboratories on a regular basis (Appendices). However, the compound does contain a small amount of Vitamin-K, less than 20 micrograms per capsule. Therefore, anyone using Coumadin (Warfarin) as a blood thinner should consult with their physician before using this product.

Several other conditions were reported for humans after the ingestion of the compound, i.e., a decrease in epileptic seizures with concurrent decrease in epileptic medications; healing of rotator cuff injuries; accelerated repair of open torsion bone fractures; less painful osteoarthritic joints; and an increase in cardiac output from 25% to 35% and then from 35% to 45% over two successive six month periods, and it has been six years after his myocardial infarction (anecdotal relative & cardiologist report).

Anecdotally regarding Parkinson disease, four Parkinson's participants have shown a cessation of cognitive decline and either a steady state condition or an increase in cognitive function taking the compound alone (caregiver/relative anecdotal observations). However, one of the participants stopped taking the compound after he had reached his cognitive goal. Unfortunately, cessation of the compound caused a slow reversion back to his decreased PD cognitive state (caregiver/relative anecdotal observations). He is starting to re-take the compound to hopefully regain the cognitive abilities he lost while off the compound.

Unfortunately, an alteration in Parkinson's motor function in PD has not been observed to date in the four Parkinson's patients taking the compound alone. In 2011, a Phase-0 study (everyone gets treated with no placebos, to test efficacy of the procedure) was performed on initially ten PD patients. The subjects took the compound for three months. Unfortunately, two participants dropped out of the study. The eight remaining participants went on to receive an inter-nasal infusion of harvested autologous stem cells into their central nervous system, as well as an aliquot of stem cells IV into their body. Motor, cognitive, affect, adjustment and caregiver variables were monitored over four time periods. Two sets of tests were given before the infusion of autologous stem cells and two sets of tests were given after the infusion of autologous stem cells.

Ten patients were initially recruited for the autologous regenerative pluripotent stem cell therapy. The study population involved subjects with PD diagnosed by Queen Square criteria. Subjects were chosen who had a Modified Hoehn and Yahr Staging 1.5 to 4 (Severe Disability), allowing for a “middle” range of the disease process. Each person selected was assessed at baseline (three months before the stem cell infusion procedure), directly before the procedure, one month after the procedure, and at four months post procedure, for a total of four assessment tests. The subjects took the compound for three months before the autologous stem cell infusion procedure and for four months after the autologous stem cell infusion procedure. At each assessment period, motor changes, as well as assess the overall improvement in cognition, affect, function, adjustment, and caregiver burden, were monitored both before and after autologous stem cell infusion. The areas that were targeted for study included the following Parkinson Assessment Criteria: (1) Motor→UPDRS-III; (2) Cognition→Symbol Digits Modality Test (SDMT), Letter Number Frequency (LNS), RBANS List learning, and Trail Making Part A and B; (3) Affect→Positive and Negative Affect Scale (PANAS), Beck Depression Inventory (BDI-II) and State Trait Anxiety (STAI); (4) Function→Functional Assessment Questionnaire (FAQ) and Schwab and England disability scale; and (5) Overall clinical improvement with the CIBIC-Plus (Clinician's Interview—Based Impression of Change Plus Caregiver Input).

Prior to study entrance, each patient underwent; (1) chart review for medication regimen, medical conditions, and laboratory values, (2) a brief physical examination, and (3) diagnostic dementia evaluations completed by the investigators. The study population involved subjects with Parkinson's disease diagnosed by Queen Square criteria. In addition, subjects were given the Mini Mental State Exam (MMSE) at entry to assure that the level of any dementia was not severe. We wanted to avoid severe dementia in the participants. We applied the RBANS Delayed Memory and selected subjects with lower index scores (typically between 70 and 100). All subjects were taking levodopa or dopamine agonists, MAO-B, or combinations. The subjects may have been on other medications, including cholinesterase inhibitors. We excluded any subject who has had DBS (deep brain stimulation) or had made plans for such. There were subjects with medical co-morbidities as well as other complications (e.g., psychiatric history, living situations, and life habits). We will attempt to adjust for these post hoc.

### Dosage

### Route and Form

The initial dosage was 500 mg of the compound, per capsule, oral administration with water on an empty stomach. At baseline (0 mo) the standard dosing regimen of the compound was initiated, i.e., one capsule of compound for 30 days, then two capsules of compound for 30 days, and then three capsules of compound for 30 days. The subject remained on three capsules for the remainder of the experiment, up to four months post autologous regenerative pluripotent stem cell infusion. Consumption of the compound lasted for seven months, during which time there was harvesting and infusion of autologous regenerative pluripotent stem cells into the intra-nasal cavity and into the circulation.

Although there are no known adverse effects associated with taking the compound, a dose reduction process was followed at the conclusion of the study. However, patients were made aware of the possibility of experiencing allergic reactions to the compound.

At seven months into the study subjects began tapering off their compound intake. Subjects who were taking three pills a day will taper to 2 pills a day. Patients continued the titrated amount of 2 pills a day for 2 weeks. Patients were then be titrated down to taking only 1 pill a day for another two weeks. At the end of the two weeks of only taking 1 pill a day, patients were officially halted from their compound intake. Any patients who chose to withdraw during the study (2) adhered to this dose reduction process.

To date, we have been able to harvest autologous pluripotent regenerative stem cells via venipuncture and to do this in a safe manner. To affect motor outcomes we used a non-invasive technique, i.e., intra-nasal infusion (Reger et al., 2008; Danielyan et al., 2009), to effectively move activated regenerative pluripotent stem cells from the peripheral vasculature into the central nervous system. In this procedure, autologous regenerative pluripotent stem cells were placed onto the olfactory mucosa and allowed to migrate into the central nervous system along olfactory projections through the cribiform plate in the ethmoid bone, migrate along the olfactory tracts into the area of the subarachnoid space, from which they could travel throughout the subarachnoid cisterns to reach damaged tissues within the brain and spinal cord.

### Intranasal+IV Infusion Protocol

1. Remove 400-ml of blood into sterile EDTA tubes.
2. Shake tubes vigorously
3. Place tubes in tube holder vertically
4. Place tubes in 4 degrees centigrade refrigeration within a range of 20 min to 96 hours
5. Remove tubes and place in laminar flow hood
6. Wipe tubes gently with 70% ethanol to sterilize outside of tubes
7. Remove tops of tubes
8. Contents of tubes have separated into ˜upper half serum and ˜lower half red blood cells & white blood cells
9. Remove upper ½ of serum and place in separate sterile tube.
10. Remove lower ½ of serum and place in separate sterile tube.
11. Spin tubes containing upper ½ of serum for 5 to 60 minutes on a desk top centrifuge to form pellet.
12. Remove 200-ml of sterile saline from within 500-ml sterile saline IV infusion solution bag
13. Take tubes with lower ½ of serum and place contents of each tube into SAME 500-ml bag of sterile saline IV infusion solution
14. Remove tubes containing upper ½ serum from centrifuge and pour off supernatant into tubes for IV infusion bag.
15. Add sterile saline from ⅛ to ⅞ volume of tube.
16. Stir (Vortex) vigorously to re-suspend pellet into saline solution
17. Centrifuge tubes containing resuspended cell pellet at high speed to wash cells of any adherent plasma (serum)
18. Pour off almost all liquid into tubes for IV-infusion bag.
19. Approximately ½-ml should remain in the pooled stem cell tube.
20. Place contents of tube into dropper bottle.
21. Have patient wash out nasal area with a sterile saline wash solution, at least ×4 with blowing nose in between each wash (need to rid nose of mucus. An alternate procedure is to use hyaluronidase to digest the mucus into smaller chains and rid the nasal chamber of the mucus.)
22. Have patient lie in Trendelenberg position, i.e., head down at 180 degrees from upright position.
23. Place ½ volume from dropper bottle into each nostril (it will sting for about 2 sec)
24. Have patient remain in Trendelenberg position from 1-60 minutes.
25. Place all remaining (lower half) serum from 1^{st} centrifuge of pelleted cells, and saline wash from subsequent pelleted cells into (see above, #'s 12, 13, 17) 500-ml of sterile saline and prepare for IV infusion.
26. IV-infuse 500-ml+ of sterile saline+additional regenerative pluripotent stem cells into vein of individual.
27. After infusion, remove needle, stop and fluid back flush into tube. Place sterile gauze over needle insertion site and wrap with tape.
   Subjects averaged 67 years old, were married, and had a normal MMSE (27), as well as RBANS Total Index (88). In the table below, ratings for overall adjustment (CIBIC and FAQ) were in the positive direction. The UPDRS and Hoehn Yahr PD ratings, key PD markers, remained about the same. The cognitive markers (presented here as Digit Symbol) declined slightly; the affect scales improved slightly.

TABLE 1

| | | Std. |
|---|---|---|
| Variable | Mean | Deviation |
| Age | 67.3 | 11 |
| MMSE | 26.8 | 2 |
| RBANS Index Ratings | 88.2 | 15 |
| PrePre CIBIC | 4. | 0 |
| Pre CIBIC | 4.2 | .7 |
| Post CIBIC | 3 | 1 |
| PrePre UPDRS | 38 | 24 |
| Pre UPDRS | 35 | 12 |
| Post UPDRS | 41 | 12 |
| PrePre Hoehn Yahr | 2.5 | .8 |
| Pre Hoehn Yahr | 2.1 | .6 |
| Post HoehnYahr | 2.5 | .2 |
| Pre Pre FAQ | 5.5 | 8 |
| Pre FAQ | 6.5 | 8 |
| Post FAQ Cargivers | 4.7 | 4 |
| Pre Pre Zarit | 24.1 | 15 |
| Pre Zarit | 19.8 | 14 |
| Post Zarit Cognitive | 20.7 | 20 |
| PrePre DigitSymbol | 31.6 | 14 |
| PreDigSymbCoding | 30.2 | 11 |
| PostDigSymb Affect | 21.8 | 9 |
| PrePre BDII | 9.5 | 7 |
| Pre BDI | 8.1 | 4 |
| Post BDI | 7.5 | 6 |

As a note, it is likely there are other positive outcomes. Once the regenerative pluripotent stem cells are circulating throughout the body, we cannot predict which damaged tissues within the body the regenerative pluripotent stem cells will migrate to and repair first. For example, a collaborator has a Parkinson's patient on one capsule of the compound per day. After four weeks, the patient's cognitive symptoms had not deteriorated, as they had in the past weeks prior to treatment. However, during that same four-week period on the compound, the patient's cardiac ejection fraction increased from 25% to 35%. And then the patient's cardiac ejection fraction increased from 35% to 45% after an additional 6 month period while on the compound. Therefore, we will be keeping note of any potential effects of compound-stimulated regenerative pluripotent stem cells on the functions of other organs throughout the body as well as those particular organs associated with Parkinson's disease.

### Preliminary Results for Parkinson's Study Jan. 24, 2012

We are in the preliminary phases with our Phase-0 Parkinson's trial. Our first contingent of Parkinson's patients (8) has been in a phase-0 trial, i.e., everyone gets treated. The patients were all moderate to low (a range of 1.5 to 4.0) with respect to their disease, on a scale of 1-10, with 10 being normal. We are still working on preliminary results (we have not broken the coding system yet), but 4 out of 8 participants are scoring about 9.0 on the scale and 4 out of 8 participants are in the range of 6 to 8 on the scale and are stable. We have one more set of tests to perform before the study is completed and we can then break the coding system. From one of the patient's physicians: “Exciting news. I've had to decrease 1 subject's medications (he insists on coming to see me in Atlanta) and he is doing very well”.

### Mesodermal

### Myocardial Infarction

Death and disability from cardiac dysfunction cost the United States nearly 750,000 lives per year. The annual cost of ischemic heart disease is approximately $100 billion [Schoen, 1999]. In most cases of myocardial infarction, an area of the heart muscle become ischemic and proceeds toward dysfunctional scar tissue, Technology leading to repair of damaged myocardium in situ would enable the medical community to change the current treatment modality of medical management to one of regeneration [Chiu, 2001]. Tremendous advances in stem cell technology indicate that it might be possible to effect repair of damaged myocardium via injection of autologous cells. Numerous cell sources have been tried in the effort to repair injured myocardium, including skeletal myoblasts [Mareli et al., 1992; Taylor et al., 1997], fetal and neonatal cardiomyocytes [Koh et al., 1993; Soonpaa et al., 1994], and embryonic stem cells [Klung et al., 1996]. The current peer strategy is to inject live naïve stem cells into and around the area of infarction. It is anticipated that the microenvironment of the heating myocardium direct the naive stem cells to integrate with the existing tissue and to differentiate to form the appropriate cellular phenotypes. Studies have shown that injection of cardiomyocytes leads to improvements in systolic performance, compliance, peri-infarct perfusion, and global ventricular function [Taylor et al., 1998; Sakai et al., 1999; Li et al., 2000]. Injected cardiomyocytes have been shown to form gap junctions with the existing tissue [Reincke et al., 1999]. These results indicate that this is a promising area for further investigation.

The focus of this study was to investigate the possibility of giving the compound alone, without stem cell infusion, to steadily increase the quantity of adult autologous regenerative pluripotent stem cells circulating through the cardiovascular system with increasing dosages of the compound. We would then determine if access to the adult regenerative pluripotent stem cells 24 hours per day for 7 days per week for 4-5 weeks per month for 12 months would affect a healing response, by measuring cardiac output in individuals having a previous myocardial infarction.

The results from a single individual were inadvertently chosen for this study. These results were derived from the aforementioned Parkinson's patient who had had a myocardial infarction six years prior to the initiation of the compound-only Parkinson study. For the previous six years, being tested every six months, this individual demonstrated a cardiac output of 25%. After cessation of Parkinson cognitive decline and stabilization of his Parkinson's symptoms, his heart showed an increase in cardiac output from 25% to 35% after a four month period. After a subsequent six month period on the compound, he demonstrated an increase in cardiac output from 35% to 45%, just taking the compound alone.

### Endodermal

### Chronic Obstructive Pulmonary Disease (COPD)

### Chronic Obstructive Pulmonary Disease (COPD) [Wikapedia, 2012]

Chronic obstructive pulmonary disease (COPD) is the occurrence of both emphysema (damage to the alveolar sacs) and chronic bronchitis (thickening of the cells lining the airways) in an individual [Wikapedia, January, 2012]. Chronic obstructive pulmonary disease results in the pulmonary airways becoming narrowed [NIH, 2010]. This leads to a decrease in the airflow to and from the lungs, causing shortness of breath (dyspnea). In clinical practice, chronic obstructive pulmonary disease is defined by its characteristically low air flow on lung function tests, i.e., FEV-1% [Nathel et al., 2007]. In contrast to asthma, which is reversible, chronic obstructive pulmonary disease is largely irreversible and usually gets progressively worse over time. In the United States, chronic obstructive pulmonary disease is the third leading cause of death. The economic burden in the United States in 2007 was $42.6 billion dollars in lost productivity and health care costs [NHLBI, 2007]. Acquisition of chronic obstructive pulmonary disease can be considered multi-factorial in that smoking, occupational exposures, air pollution, genetics, autoimmune diseases, repeated lung diseases, and a diet high in cured meats (exposure to sodium nitrates) are all contributing factors to development of the disease [MedicineNet.com, 2012; Young et al., 2009; Rennard and Vestbo, 2006; Devereux, 2006; Halbert et al., 2006; Kennedy et al., 2007; MedlinePlus Encyclopedia 000091; Rutgers et al., 2000; Feghali-Bostwick et al., 2008; Lee et al., 2007].

### Testing for COPD:

The diagnosis of COPD is confirmed by spirometry [Rabe et al., 2007], a test that measures the forced expiratory volume in one second (FEV₁), which is the greatest volume of air that can be breathed out in the first second of a large breath. Spirometry also measures the forced vital capacity (FVC), which is the greatest volume of air that can be breathed out in a whole large breath. Normally, at least 70% of the FVC comes out in the first second (i.e. the FEV₁/FVC ratio is >70%). A ratio less than normal defines the patient as having COPD. More specifically, the diagnosis of COPD is made when the FEV₁/FVC ratio is <70% [Nathel et al., 2007]. The GOLD criteria also require that values are after bronchodilator medication has been given to make the diagnosis, and the NICE criteria also require FEV1% [Nathel et al., 2007]. According to the ERS criteria, it is FEV1% predicted that defines when a patient has COPD, that is, when FEV1% predicted is <88% for men, or <89% for women [Nathel et al., 2007].

Spirometry can help to determine the severity of COPD [Rabe et al., 2007]. The FEV₁ (measured after bronchodilator medication) is expressed as a percentage of a predicted “normal” value based on a person's age, gender, height and weight.

| Severity of COPD | |
|---|---|
| (GOLD scale) | FEV₁ % predicted |
| Mild (GOLD 1) | ≧80 |
| Moderate (GOLD 2) | 50-79 |
| Severe (GOLD 4) | 30-49 |
| Very Severe (GOLD 5) | <30 or chronic respiratory |
| | failure symptoms |

The severity of COPD also depends on the severity of dyspnea and exercise limitation. These and other factors can be combined with spirometry results to obtain a COPD severity score that takes multiple dimensions of the disease into account [Cell et al., 2004].

### Assessment

There is NO known cure for COPD.

### Prognosis

COPD usually gradually gets worse over time and can lead to death. The rate at which it gets worse varies between individuals. The factors that predict a poorer prognosis are the following [Rabe et al., 2007].
Severe airflow obstruction (low FEVₛ)
Poor exercise capacity
Shortness of breath
Significantly underweight or overweight
Complications like respiratory failure
Continued smoking
Frequent acute exacerbations

Five patients were initially recruited for the autologous regenerative pluripotent stem cell therapy. The study population involved subjects with chronic obstructive pulmonary disease. We have chosen subjects that had a FEV1% of less than 30% (Very Severe). Each person selected was assessed at baseline (before the study started) and at termination of the study. The subjects took the compound for three to twelve months before the autologous stem cell infusion procedure, i.e., nebulization with accompanying IV infusion. At the assessment period, we evaluated the change in percentage of FEV 1.

Prior to study entrance, each patient underwent; (1) a chart review for medication regimen, medical conditions, and laboratory values, and (2) a brief physical examination. The study population involved subjects with COPD diagnosed by multiple criteria, including FEV1% of less than 30%.

### Dosage

### Route and Form

The initial dosage was 500 mg of the compound, per capsule, oral administration with water on an empty stomach. At baseline (0 mo) the standard dosing regimen of the compound was initiated, i.e., one capsule of compound for 30 days, then two capsules of compound for 30 days, then three capsules of compound for 30 days, then four capsules of compound for 30 days, then five capsules of compound for 30 days, then six capsules of compound for 30 days, then seven capsules of compound for 30 days, then eight capsules of compound for 30 days, then remain on eight capsules of compound for the remainder of the study. Consumption of the compound lasted for 12 months, during which time there was harvesting and infusion of regenerative pluripotent stem cells by nebulization into the lungs and IV infusion into the circulation. Although in the Parkinson study, a dose reduction process occurred, all participants of the COPD study elected to remain on compound until further stem cell treatments could be performed. Patients were made aware of the possibility of experiencing allergic reactions to the compound.

### Nebulization+IV Infusion Protocol

1. Remove 400-ml of blood into sterile EDTA tubes.
2. Shake tubes vigorously
3. Place tubes in tube holder vertically
4. Place tubes in 4 degrees centigrade refrigeration for a minimum of 48 hours
5. Remove tubes and place in laminar flow hood
6. Wipe tubes gently with 70% ethanol to sterilize outside of tubes
7. Remove tops of tubes
8. Tubes have separated into ˜upper half serum and ˜lower half red blood cells & white blood cells
9. Remove upper ½ of serum and place in separate sterile tube.
10. Remove lower ½ of serum and place in separate sterile tube.
11. Spin tubes containing upper ½ of serum for 15 min on desk top centrifuge to form pellet.
12. Remove 200-ml of sterile saline within 500-ml sterile saline IV infusion solution
13. Take tubes with lower ½ of serum and place contents of each tube into SAME 500-ml bag of sterile saline IV infusion solution
14. Remove tubes containing upper ½ serum from centrifuge and pour off supernatant into tubes for IV infusion bag.
15. Add sterile saline at ½ volume of tube.
16. Stir (Vortex) vigorously (10×1-second pulses on setting #6) to re-suspend pellet into saline solution
17. Centrifuge tubes containing resuspended cell pellet at high speed to wash cells of any adherent plasma (serum)
18. Pour off liquid into tubes for IV-infusion bag.
19. Approximately 2-ml should remain in the pooled tube.
20. Place contents of tube into nebulization container.
21. Turn on nebulizer.
22. Have patient breathe in deeply and hold breath for 2 to 5 seconds before exhaling.
23. Repeat step #22 until all stem cell solution is gone.
24. Turn off and disconnect nebulizer.
25. Place all remaining (lower half) serum from 1^{st} centrifuge of pelleted cells, and saline wash from subsequent pelleted cells into (see above, #'s 12, 13, 17) 500-ml of sterile saline and prepare for IV infusion.
26. IV-infuse 500-ml+ of sterile saline+additional pluripotent stem cells into vein of individual.
27. After infusion, remove needle, stop and fluid back flush into tube. Place sterile gauze over needle insertion site and wrap with tape.

### Preliminary Results

We have the results from two participants thus far, as shown below.
FEV1 is forced expiratory volume in 1 second.
FVC is forced vital capacity.

| HF298: 3/28/11 | 12/9/11 |
|---|---|
| FEV1- 0.34 15% | FEV1- 0.50 24% |
| | (Predicted 2.11 liters) |
| FVC- 0.84 30% | FVC- 1.34 47% |
| | (Predicted 2.86 liters) |

Treatment for HF298 was completed Jul. 20, 2011, after approximately nine months on the compound, while her testing for FEV1 percentage and FVC were not performed until Dec. 9, 2011. As shown, the percentage of forced expiratory volume in 1 second, or FEV1%, for HF298 increased by 60% from her initial FEV1% of 15%. HF298's forced vital capacity, FVC, increased from her initial value of 30% prior to stem cell infusion to a value of 47%. This is an increase of 56.6% from her original value. Since there is no known cure for COPD and the individuals with this disease only spiral downwards towards zero and death, this result is a significant improvement in COPD therapy.

| 6/28/2011 | 10/11/2111 | 01/17/2012 |
|---|---|---|
| FEV1. 66% | FEV1. 73% | FEV1. 72% |
| FVC. 55% | FVC. 60% | FVC. 60% |

While our second participant showed a percentage difference of 7% for FEV1 and a 5% difference for FVC, these are still significant values, since the expected outcome is a downward spiral leading to death.

### BRIEF DESCRIPTION OF THE DRAWINGS

### Figure Descriptions

FIG. 1. Lineage Map of unidirectional cell differentiation pathway for cells derived from the embryonic zygote and cells derived from adult blastomeric-like stem cells. The blastomeric-like stem cell is equivalent to blastocyst; the epiblast-like stem cell is equivalent to epiblast; and germ layer lineage ectoderm, mesoderm, and endoderm stem cells, are equivalent to ectoderm, mesoderm, and endoderm respectively. Reprinted with permission from Adult Stem Cells, by H. E. Young and A. C. Black, Jr., FIG. 1, Anatomical Record Part A 267A:75-102, 2004. Wiley-Liss, Inc.

FIG. 2. Scanning electron micrograph of embryo, somewhere between 8-cell stage and morula. Note presence of three pairs of blastomeric-like stem cells (BLSCs) and one epiblast-like stem cell (ELSC). Size approximation same as seen with flow cytometry (BLSCs, 0.2 to 2 microns in size and ELSC, 6 to 8 microns in size.

FIG. 3. Scanning electron micrograph of embryo, somewhere between 8-cell stage and morula. Note nine large cells, called blastomeres, which when disrupted, separated and plated, become embryonic stem cells.

FIG. 4. Totipotent blastomeric-like stem cells (BLSCs) are small round cells, ranging in size from 0.2 microns to 2.0 microns in size. BLSCs in size range of 1.0 to 2.0 microns attach to the dish in a bipolar-type fashion. The 0.2 to 1.0 micron BLSCs and the 1.0 to 2.0 micron BLSCs are carcinoembryonic antigen-cell adhesion molecule-1 positive, stage specific embryonic antigen (SSEA) negative, and Trypan blue negative. The figure was taken with a Nikon Cool-pix camera mounted onto a Nikon TMS inverted phase contrast and bright field microscope with phototube. Original magnification was 200×, which was electronically magnified to 800×, and then magnified to 1600× by Adobe Photoshop 7.0.

FIG. 5. Two BLSCs (small arrow) in photograph. Of the two BLSCs in the photograph one is in prophase and the other is in metaphase (see chromosomes along metaphase plate). Also note presence of single ELSC (large arrowhead), and a portion of a progenitor cell (asterisk), Magnification of original photograph, 200×, electronically modified to 1600×.

FIG. 6. Flow cytometry of human (HM0001) blastomeric-like stem cells. BLSCs are located in an area normally assumed as containing trash. Yet when this material stained with propidium iodide (data not shown), 95% of the material stains for intact nucleated cells. Size range in this area is 0.2 to 2.0 microns.

FIG. 7. Blastomeric-like stem cells (BLSCs) harvested from male Sprague-Dawley out bred rat 0001 and propagated from a single cell derived by serial dilution clonogenic analysis, then genomically labeled with LacZ to synthesize the beta-galactosidase gene product. Clone designated as Scl-44beta. Plated in culture in the presence of insulin at 2 micrograms per ml and in the absence of inhibitors, no induction occurs. A: Bright field microscopy, positive staining for cell-surface marker CEA-CAM-1. B-L is phase contrast microscopy to show unstained cells in field of view. B: FORSE-1 negative for neuroectoderm; C: 8A2 negative for neurons; D: S-100 negative for neurons; E: CNPase negative for glial cells; F: 40E-C negative for ependymal cells; G: SV2 negative for neuro vesicles; H: VM-1 negative for keratinocytes; I: IA4 negative for smooth muscle cells; J: CIIC1 negative for type-II collagen which is indicative of cartilage; K: MPIII negative for osteopontine which is indicative of bone; and WV1D1 for osteocalcin which is indicative of calcified tissues, i.e., bone. Original magnification 200×.

FIG. 8. Blastomeric-like stem cells (BLSCs) harvested from male Sprague-Dawley out bred rat 0001 and propagated from a single cell derived by serial dilution clonogenic analysis, then genomically labeled with LacZ to synthesize the beta-galactosidase gene product. Clone designated as Scl-44beta. Induction of ELSCs (B), spermatogonia (T) and representative ectodermal, mesodermal and endodermal cell types (C-S). All Pictures—Bright field microscopy; A & B show positive staining after incubation in buffer only; A, CEA-CAM-1+ positive for BLSCs; B, SSEA+ marker for ELSCs; C, FORSE-1 positive for neuroectodermal cells; D, S-100 positive for neurons with 100 nm filaments; E, RT-97 positive for neurons; F, DOPA—positive for neurons containing dopamine; G, CNPase, positive for glial cells (Oligodendrocytes and astrocytes); H, GFAP, glial fibrillary acidic protein—positive for glial cells; I, RIP, positive for glial cells; J, 40E-C, positive for ependymal cells lining the CNS; K, MF-20—representative of skeletal muscle cells; L, CIIC1, type-II collagen representative of cartilage; M, RAFP, rat alpha feto-protein, representative of endodermal progenitor cells; N, Pax-6, representative of endodermal cells; O, OC5, representative of liver cells; P, H1, representative of hepatocytes; Q, INS, representative of pancreatic beta cell containing insulin; R, glucagon-containing cells, representative of pancreatic alpha cells; S, SOMA, representative of somatostatin containing delta cells; T, DH-TuAG1, representative of cells of spermatogonia. Original magnification of 200× Putative stem cells cultured in general induction medium demonstrate CEA-CAM-1(+) staining and transitory SSEA-4(+) staining prior to phenotypic expression markers indicative of 66 cell types from all three germ layer lineages: ectoderm, mesoderm, endoderm; and spermatogonia (suggests cells are totipotent stem cells=BLSCs).

FIG. 9. HM001, adult human stem cells isolated, stained 1:1 with 0.4% Trypan blue solution, diluted 1:1000 with sterile saline and mounted onto a hemocytometer, magnification 200×. Note cells that are white “glowing” spheres=ELSCs; cells that are spherical and solid blue=large-BLSCs or small-BLSCs.

FIG. 10. HM0001 isolated adult human stem cells from blood after stimulation to multiply in vivo by ingestion of the compound. Cells stained 1:1 with 0.4% Trypan blue solution, diluted 1:1000 with sterile saline and mounted onto a hemocytometer, magnification 200×. Note cells that are white “glowing” spheres=ELSCs are Trypan blue negative; cells that are spherical and solid blue=large-BLSCs and small-BLSCs and are cells that are Trypan blue positive. Original magnification 200×. B. Cells stained with CEA-CAM-1 antibody, diluted 1:1000 with sterile saline and mounted onto a hemocytometer, magnification 200×. Note very small cells that display CEA-CAM-1 throughout=small Blastomeric-Like stem cells (BLSCs), which are 0.2 to 1.0 microns in size on flow cytometry of live cells.

FIG. 11. Karyotypic analysis of Sprague-Dawley out bred rat (RM0001) BLSCs (Scl-44b) demonstrating 42 chromosomes in the diploid state.

FIG. 12. A mixed culture of multi-confluent layers of Sprague-Dawley out bred rat (RM001) BLSCs (Scl-44beta) and ELSCs (Scl-40beta), grown on a type-I collagen substratum.

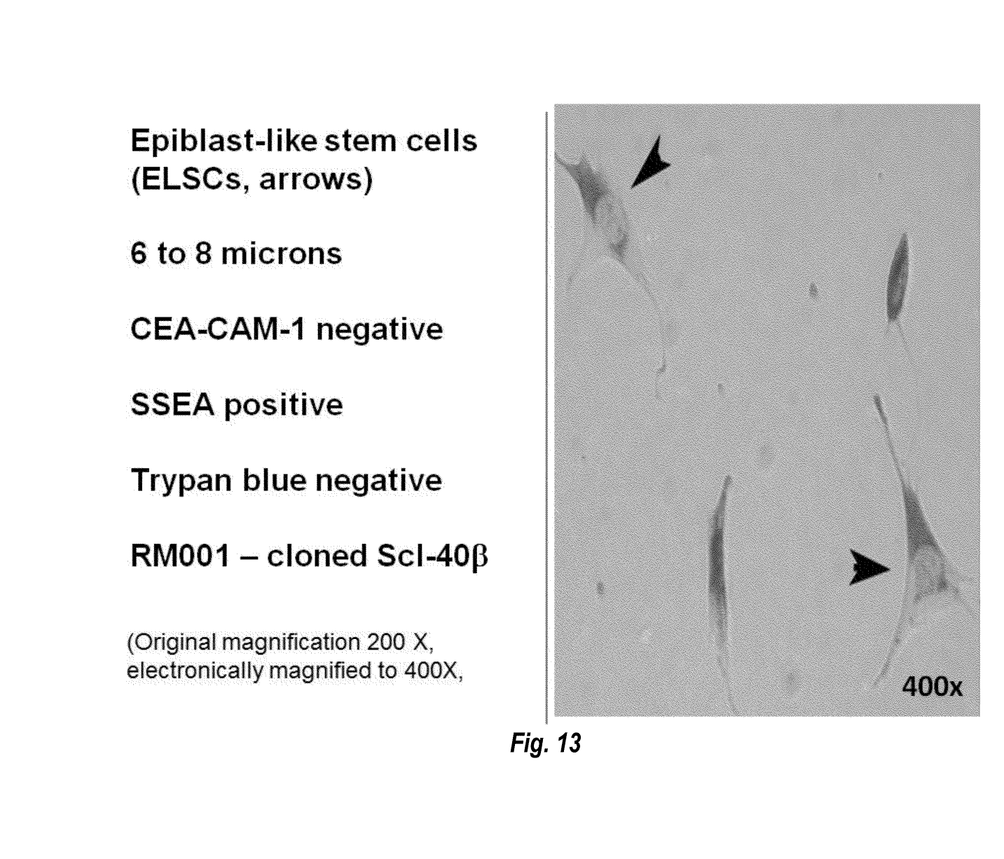

FIG. 13. Plated ELSCs demonstrating cells that are predominantly multipolar in appearance. Positive staining for SSEA, they are 6 to 8 microns in size by flow cytometry of live cells. Staining pattern is CEA-CAM-1 negative, SSEA positive, and Trypan blue negative. The cells were cloned from and adult male rat, number 0001 (RM0001).

FIG. 14. Epiblast-like stem cells (ELSCs) harvested from male Sprague-Dawley out bred rat (RM0001) and propagated from a single cell derived by serial dilution clonogenic analysis, then genomically labeled with LacZ to synthesize the beta-galactosidase gene product. Clone designated as Scl-40-beta. Induction of representative ectodermal cell types: (A-P)—Brightfield microscopy; A, negative for CEA-CAM-1 (BLSC); B, positive for SSEA (ELSC); C, positive for Rat-401 (Neuro-ectoderm); D, positive for MAB353 (neuroectoderm); E, positive for FORSE-1 (neuroectoderm); F, positive for 8A2 (neuron); G, positive for RT-97 (neuron); H, positive for S-100 (neuron); I, positive for tyrosine hydroxylase Neurotransmitter for neuron); J, positive for dopamine (neuron); K, positive for SV2 (neurovesicle); L positive for TIP (glial cell); M, positive for CNPase, positive for glial cells; N, positive for GFAP (glial cell); 0, positive for 40E-C (ependymal lining cells); and P, positive for VM-1 (keratinocytes).

FIG. 15. Epiblast-like stem cells (ELSCs) harvested from male Sprague-Dawley out bred rat (RM0001) and propagated from a single cell derived by serial dilution clonogenic analysis, then genomically labeled with LacZ to synthesize the beta-galactosidase gene product. Clone designated as Scl-40□. Induction of representative mesodermal and endodermal cell types: (A-T)—Bright field microscopy: A, F5D, positive for skeletal; B, A4,74, positive for skeletal; C, IA4, positive for smooth muscle alpha actin; D, MAB3252, positive for cardiac muscle; E, CIIC1, positive for cartilage; D1-9, positive for type-9 collagen in cartilage; G, MP111, positive for osteonectin in bone; H, RAFP, positive for rat endodermal feta-protein, positive for endodermal cells; I, HESA, positive for GI-epithelial cells; J, pancreatic alpha cells, positive for glucagon cells; K, insulin, positive for beta-pancreatic cells; L, glucagon, positive for pancreatic delta cells; M, 151-IgG, positive for gastrointestinal epithelial cells; N, positive for liver progenitor cells: oval cells, biliary cells, and liver epithelial cells; O, OC4, positive for LP OvC BE canalicular cells; P, OC10, positive for LP, OvC, BE, and CC cells; Q, H1, positive for hepatocytes; R, H4, positive for hepatocytes; S, DPPIV, positive for LP, OvC, BE, and CC cells; and T, HA4c19, positive for pancreatic ductal cells.

Putative stem cells cultured in general induction medium demonstrate CEA-CAM-1 (−) staining, but SSEA-4(+) staining prior to staining for phenotypic expression markers indicative of 63 cell types from all three germ layer lineages: ectoderm, mesoderm, endoderm, but no spermatogonium (suggests cells are pluripotent stem cells=ELSCs).

FIG. 16. Molecular analysis of telomerase activity in a Lac-Z-transfected postnatal rat epiblast-like stem cell clone, designated Scl-40□ and a Lac-Z transfected rat mesodermal clone, designated Rat-A2A2□. A: Polyacrylamide gel electrophoresis of telomerase activity in pluripotent stem cells at 254 population doublings (Scl-40b) and 151 population doublings (A2A2□) were utilized. Cells were propagated, harvested by trypsin released (Young et al., 1999) and processed for telomerase activity as described by the manufacturer (TRAPeze Assay, Intergen). Lane 1, molecular weight controls; lane 2, blank; lane A−, extraction buffer (negative control); lane A+, extract of telomerase positive cells (positive control); lane B−, heat inactivated test extract of GLMesoSCs; and lane B+, test extract of GLMesoSCs; lane C−, heat inactivated extract of ELSC; lane C+, test extract of ELSC.

FIG. 17. Karyotypic analysis of Sprague-Dawley out bred rat (RM0001) ELSCs (Scl-40□) demonstrating 42 chromosomes in the diploid state, normal number of chromosomes for a rat.

FIG. 18. Adult rat heart (RM1021), animal euthanized, tissue harvested, fixed in a glutaraldehyde-paraformaldehyde-glucose fixative, cryosectioned at 7 microns and stained with antibodies for: A, ELSCs (SSEA+); B, BLSCs (CEA-CAM-1+); C, smooth muscle (IA4+) as positive procedural control, and D, no primary antibody as negative procedural control.

FIG. 19. Adult rat cerebral cortex (RM1021) cryosectioned at 7 microns and stained with antibodies for: A, SSEA for ELSCs (positive cells noted with arrowheads); B, CEA-CAM-1 for BLSCs, dark brown spots with some noted with arrowheads; C IA4 for smooth muscle in wall of blood vessels as positive procedural control; and D, no primary antibody as the negative procedural control.

FIG. 20. Adult porcine (RM0010) pancreas, bright field microscopy. A. SSEA positive stained cells overlying both islets and acinar cells. B. CEA-CAM-1 positively stained cells overlying pancreatic islets and acinar cells. C. IA4 positive staining for smooth muscle alpha-actin in small blood vessels within pancreas, positive procedural control. D. No primary antibody, negative procedural control.

FIG. 21. Antibody staining within dermis and hypodermis of adult skin from male porcine, PM0010. A.CEA-CAM-1+ cells within loose fibrous connective tissues surrounding unilocular adipose (fat) cells in hypodermis, 40× mag. B.CEA-CAM-1+ cells in loose fibrous connective surrounding unstained blood vessel, 200× mag. C. SSEA+ cells in loose fibrous connective tissue surrounding blood vessel in border region between reticular dermis and hypodermis, 100× mag. D: IA4+ staining of smooth muscle alpha-actin in tunica media of blood vessel (positive procedural control). Note unstained RBCs in lumen of vessel, 200× mag.

FIG. 22. Normal non-regenerating lung tissue from adult rat RM1021. Red-stained CEA-CAM-1+ cells and SSEA+ cells are located within the smooth muscle layer of the blood vessels and structures within the bronchial tree. A: CEA-CAM-1+ cells in tunica media of a vein, 200× mag. B: CEA-CAM-1+ cells within smooth muscle layer of alveolar ducts (AD) and bronchiole (B), 200× mag. C: SSEA+ cells in tunica media (smooth muscle layer) of artery (A) and vein (V), 200× mag. D: SSEA+ cells in smooth muscle wall of bronchial (B) and artery (A), 200× mag.

FIG. 23. Adult mouse bone marrow obtained from flushing out bone marrow with sterile saline and then smearing bone marrow on glass slide, fixation with glutaraldehyde-paraformaldehyde-glucose fixative. Slides were stained with appropriate primary antibody to identify cell types. A.CEA-CAM-1 positive cells (BLSCs). B. SSEA positive cells (ELSCs) C. IA4 smooth muscle alpha actin in tunica media of arterioles. D. No primary antibody, negative procedural control.

FIG. 24. Adult porcine (PM0010) kidney stained with appropriate primary antibodies to identify cells. SSEA positive cells (ELSCs) B.CEA-CAM-1 positive cells (BLSCs) C. IA4 positive staining for smooth muscle alpha actin in the tunica media of blood vessel, positive procedural control. D. No primary antibody, negative procedural control. Arrows outline glomeruli in kidney.

FIG. 25. Adult rat skeletal muscle (RM1025), euthanized after tissue trauma, and tissue harvested, fixed in a glutaraldehyde-paraformaldehyde-glucose fixative, cryosectioned at 7 microns, mounted on glass slides, and stained with appropriate antibodies. A. SSEA positive (ELSCs) with nerve fiber connective tissues. B.CEA-CAM-1 positive (BLS) staining within tunica intima of blood vessels. C. IA4 positive staining for smooth muscle alpha-actin in the tunica muscularis of medium and small blood vessels within the permysium of skeletal muscle, positive procedural control. D. No primary antibody, negative procedural control.

FIG. 26. Adult rat testis (RM1021), tissue harvested, fixed in a glutaraldehyde-paraformaldehyde-glucose fixative, cryosectioned at 7 microns, mounted on glass slides, and stained with appropriate antibodies. A. SSEA positive cells (ELSCs) surrounding outside of seminiferous tubules (arrows) as well as within the innermost layer of seminiferous tubules. B.CEA-CAM-1 positive staining (BLSCs) completely of seminiferous tubules with heaviest staining along the inside border of the tubules. C. IA4 positive staining of smooth muscle alpha actin with the tunica media of blood vessels within the testis, positive procedural control. D. No primary antibody, negative procedural control.

FIG. 27. Adult rat ovary (RF1022). A.CEA-CAM-1 positive staining in area of ovum inside tertiary follicle. B.SSEA positive cells interspersed among granulosa cells of Graffian follicle. C. (BLANK) IA4 positive staining for smooth muscle alpha-actin in the tunica media of blood vessels within the ovary, positive procedural control. E. No primary antibody, no staining present within ovary, negative procedural control.

FIG. 28. Adult female rat (RF1022) fallopian tube harvested from euthanized rat, fixed in glutaraldehyde-paraformaldehyde-glucose fixative, cryosectioned at 7 microns, and stained with appropriate antibodies. A.SSEA positive ELSCs with area of ampulla of fallopian tube. B.CEA-CAM-1 positive BLSCs in the connective tissues of ampulla region of the fallopian tube. C. IA4 positive smooth muscle alpha-actin in the smooth muscle within the wall of the fallopian tube, positive procedural control. D. No primary antibody, negative procedural control.

FIG. 29. Adult equine blood (EG0001) smeared on a glass slide, fixed with a glutaraldehyde-paraformaldehyde-glucose fixative and stained with the appropriate antibodies. A. CEA-CAM positive BLSCs; B. SSEA positive ELSCs; and C. No primary antibody as negative procedural control.

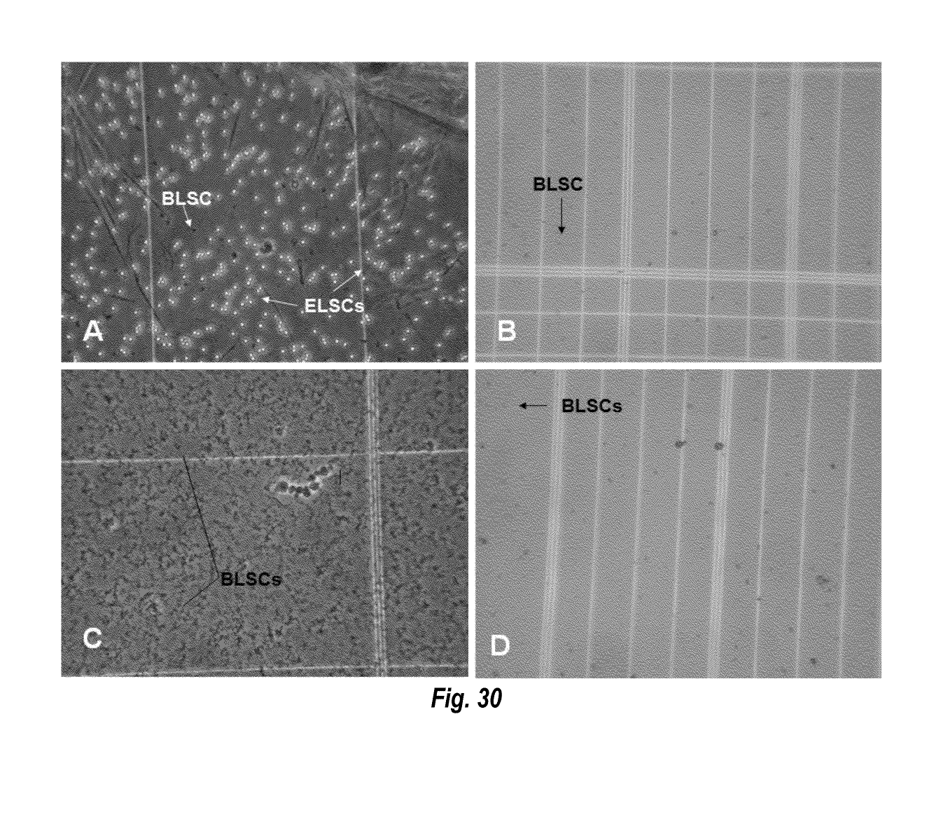

FIG. 30. Felines and Canines stained plasma fractions. A. Feline plasma fraction stained with 0.4% Trypan blue. BLSCs are small dark round circles and ELSCs have glowing white nuclei, 100× mag. B. Feline plasma fraction stained with CEA-CAM-1. BLSCs are small dark-red round circles, 100× mag. C. Canine plasma fraction stained with 0.4% Trypan blue. BLSCs are small dark round circles and Tr-BLSCs are structures with a dark periphery and a clear center, 100× mag. D. Canine plasma fraction stained with CEA-CAM-1. BLSCs are small dark-red round circles and Tr-BLSCs are structures with a dark-red periphery and a clear center, 100× mag.

FIG. 31. Ovine (Sheep) and Caprine (Goat) stained plasma fractions. Ovine plasma fraction stained with 0.4% Trypan blue. BLSCs are small dark round circles, with a dark periphery and a clear center, 100× mags. B. Ovine plasma fraction stained with CEA-CAM-1. BLSCs are small dark-red round circles, and a clear center, and ELSCs are unstained, 100× mag. C. Caprine plasma fraction stained with 0.4% Trypan blue. BLSCs are small dark round circles, 100× mag. D: Caprine plasma fraction stained with CEA-CAM-1. BLSCs are small dark-red round circles 100× Mag.

FIG. 32. Bovine and Equine stained plasma fractions. A. Bovine plasma fraction stained with 0.4% Trypan blue. BLSCs are small dark round circles and ELSCs have white glowing nuclei with phase contrast microscopy, 100× mag. B. Bovine plasma fraction stained with CEA-CAM-1. BLSCs are small dark-red round circles, 100× mags. C. Equine plasma fraction stained with 0.4% Trypan blue. BLSCs are small dark round circles, 100× mags. D. Equine plasma fraction stained with CEA-CAM-1. BLSCs are small dark-red round circles, 100× mags.

FIG. 33. Isolation protocol for differentiated cells, progenitor cells, germ layer lineage stem cells, epiblast-like stem cells, and blastomeric-like stem cells. The flow chart is based on cryopreservation, and cloning by repetitive single cell clonogenic analysis or cell sorting using unique cell surface markers to separate GLSCs, ELSCs, and BLSCs.

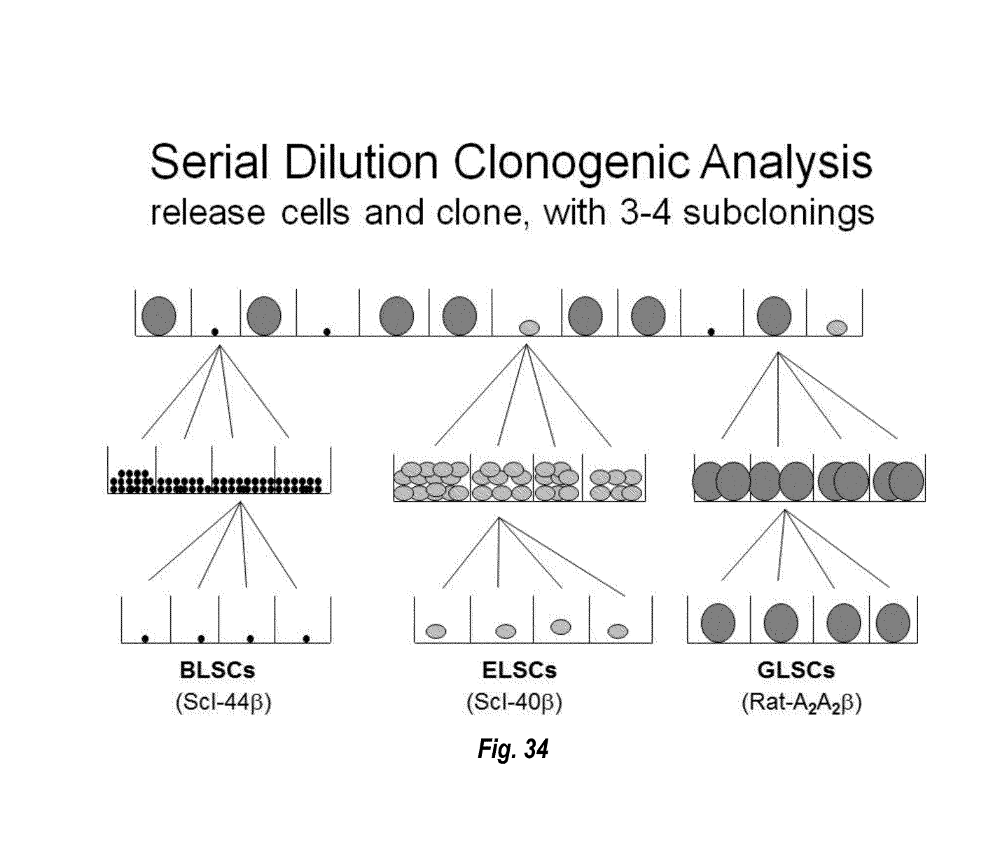

FIG. 34. Serial dilution single cell clonogenic analysis for BLSCs, ELSCs, and GLSCs. Cell suspension isolated from the skeletal muscle of outbred adult Sprague-Dawley rat. RM0001. Cell suspension was diluted to single cell concentration of one cell per 5 microliters of medium. Single cells in 5 microliters of conditioned medium were plated per well of 96-well plates, grown post confluence, replated as single cells per well, grown post confluence, 3-4 times to insure that population of cells came from a single cell [Young et al., 2001, 2004a; Young and Black, 2005a].

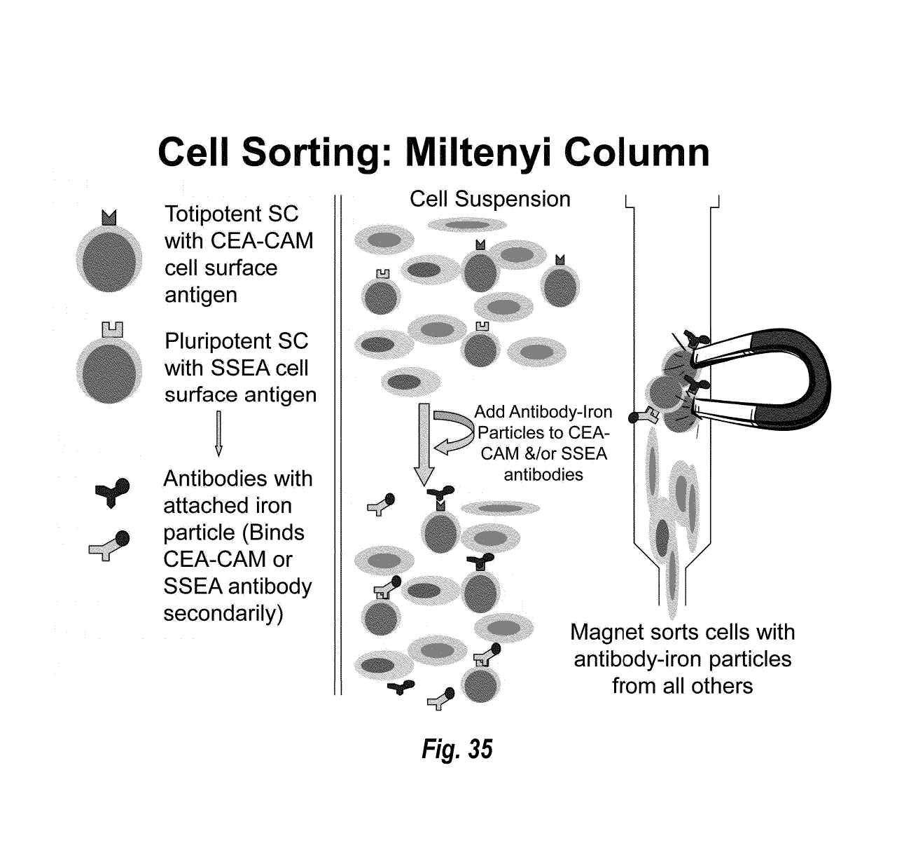

FIG. 35. Miltenyi sort of ELSC (SSEA) and BLSC (CEA-CAM-1) isolation from remaining cell types from stem cell harvest. Yields routinely in the 95-98% purity range.

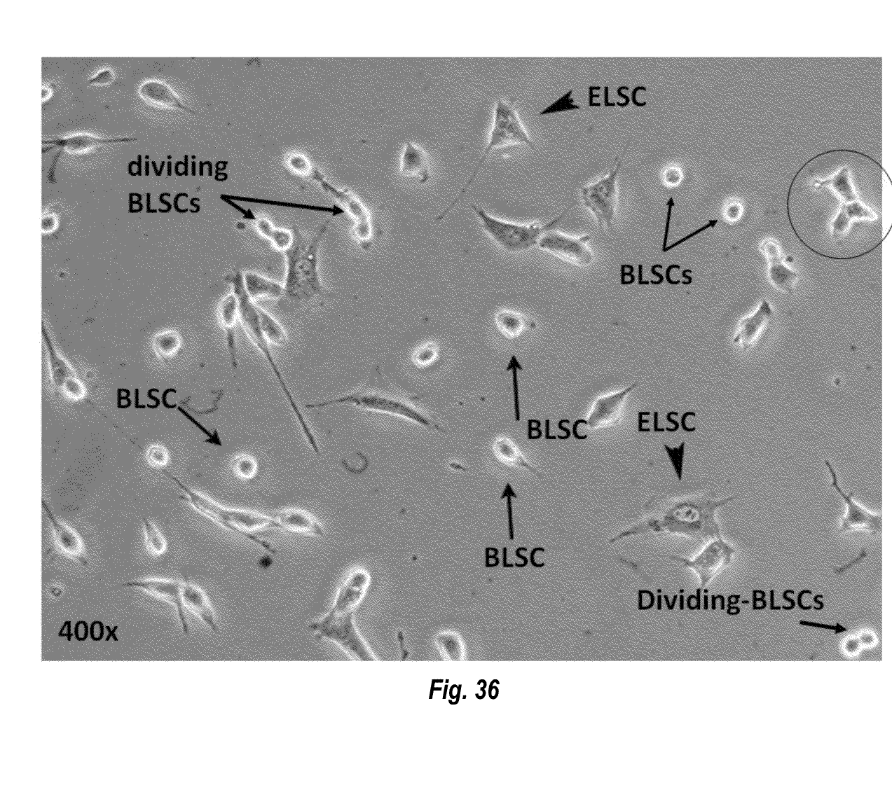

FIG. 36. Plated cells after first Miltenyi sort. Remaining cells are attached and dividing ELSCs and BLSCs, original magnification at 400×.

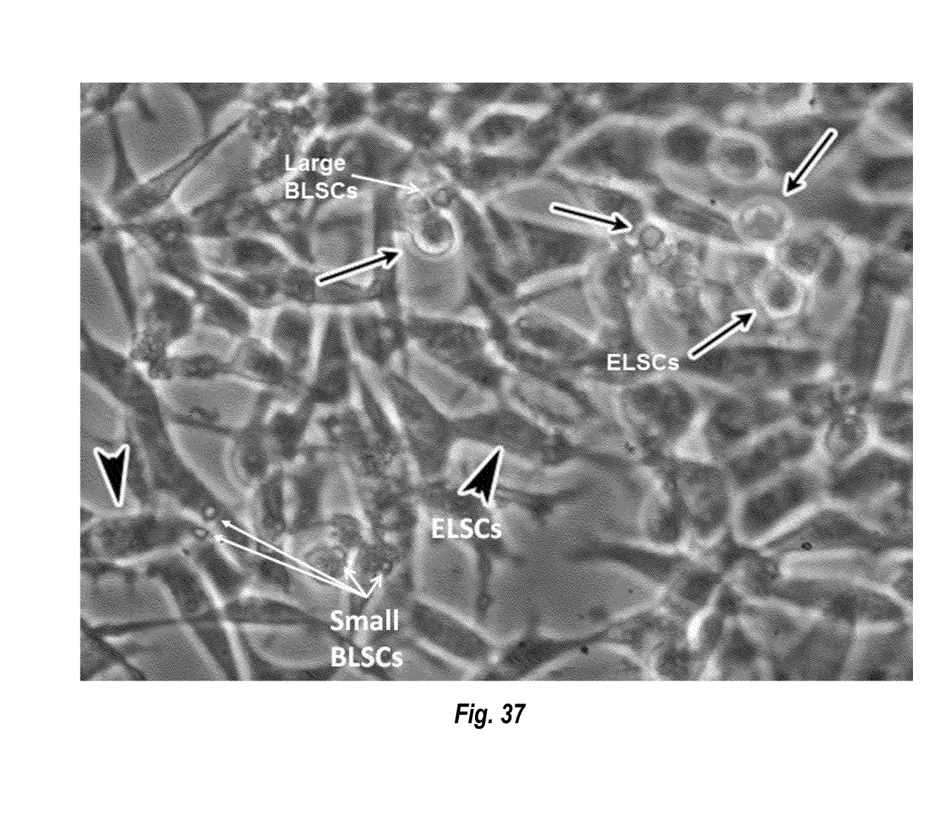

FIG. 37. Combination of freshly isolated epiblast-like stem cells (ELSC), transitional blastomere-like stem cells (Tr-BLSCs), large-blastomeric-like stem cells (large-BLSCs), and small-blastomeric-like stem cells (small-BLSCs).

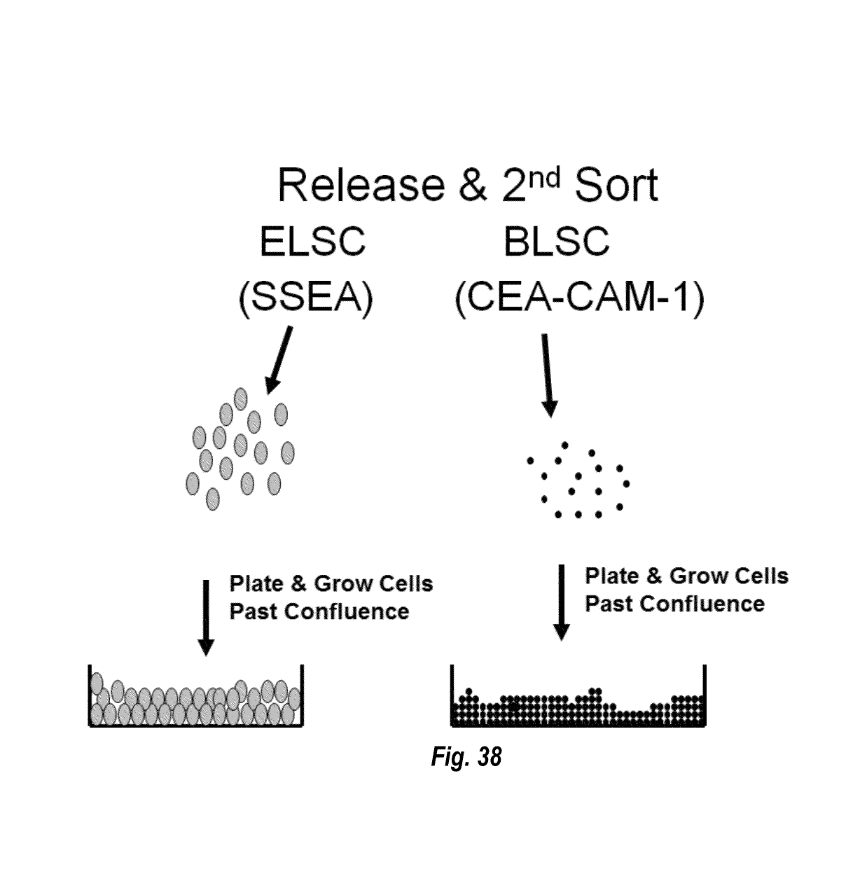

FIG. 38. Separation of BLSCs from ELSCs by Miltenyi sort using SSEA and CEA-CAM-1. First, S SEA-bound iron particles were incubated with cell mixture and passed over Miltenyi column. Only SSEA-bound ELSCs and some Tr-BLSCs adhered to column, while non-SSEA—bound cells appeared in the eluant. Next, the eluant was incubated with CEA-CAM-1-bound iron particles. Only CEA-CAM-1-bound BLSCs and residual Tr-BLSCs adhered to column, while potentially non-CEA-CAM-1-bound cells appeared in eluant. Eluant was tested for cells, none were present. Only 2-4% of cells (Tr-BLSCs) demonstrated cross-over. Purity from Miltenyi sorts averaged 96-98%.

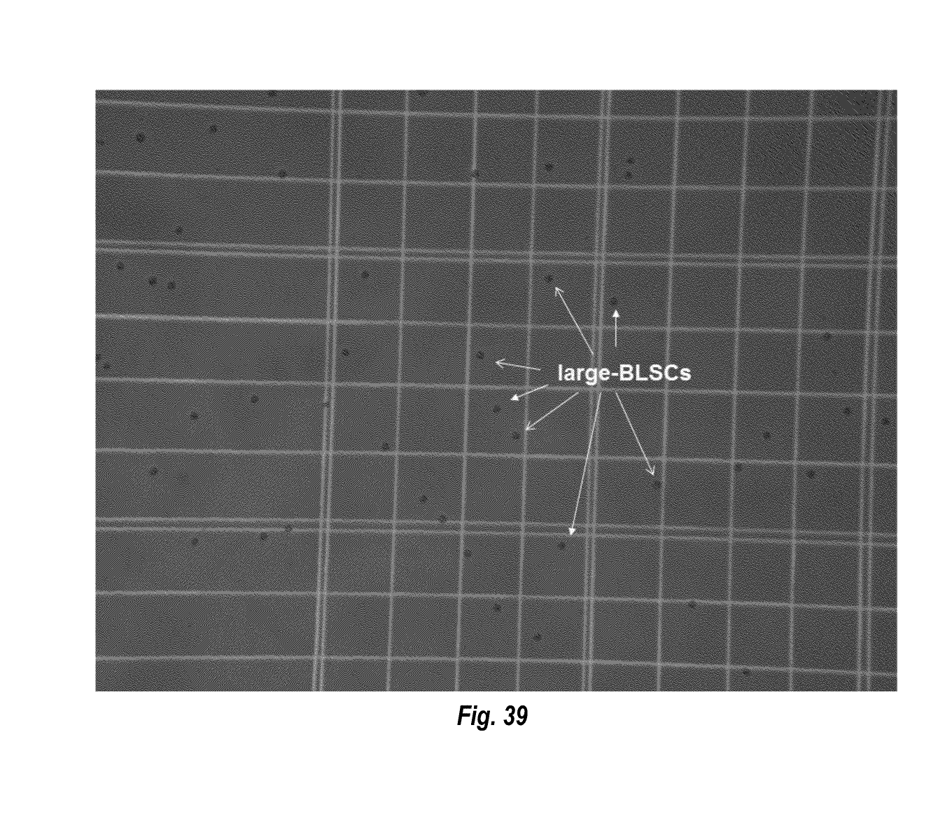

FIG. 39. Extracted large Trypan blue-positive BLSCs (1.0 to 2.0 microns in size) from adult porcine blood (PM0012) and stained with 0.4% Trypan blue. Small round dots on slide (arrows) are Trypan blue-positive large-BLSCs.

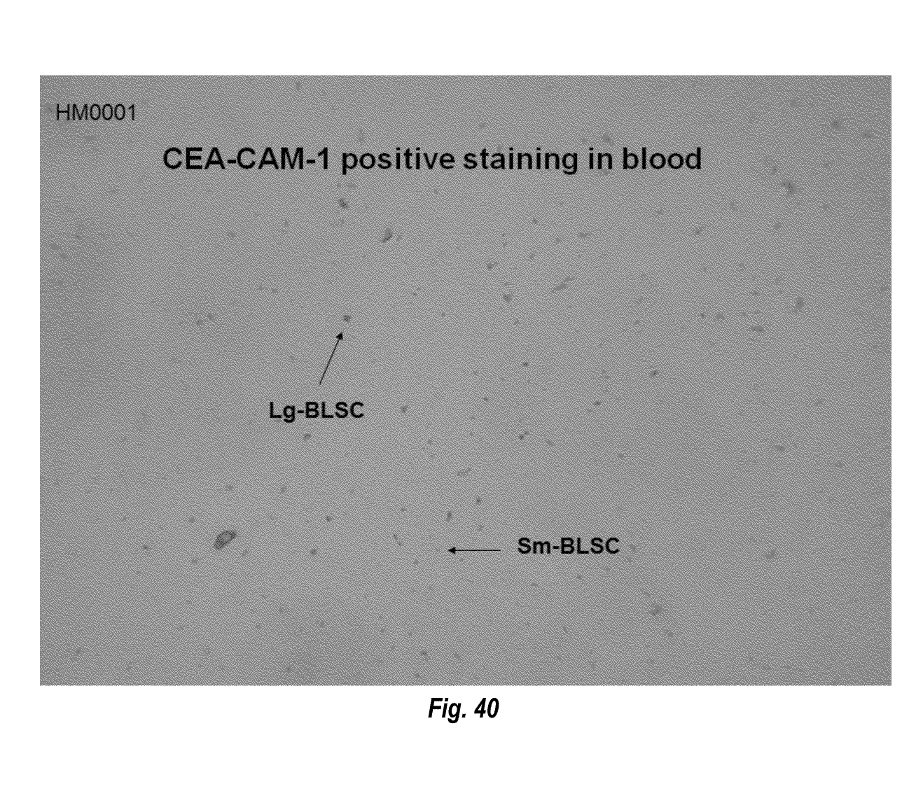

FIG. 40. HM001, adult human stem cells isolated from blood. Cells stained with CEA-CAM-1, diluted to 1:1000. and plated onto hemocytometer. Tr-BLSCs and BLSCs are CEA-CAM-1+.

FIG. 41. Creation of a Parkinson disease model by stereotactically injecting a neurotoxin, 6-hydroxydopamine, into the substantia nigra on one side of the brain.

FIG. 42. Normal corpus striatum of adult rat brain (sham control hemi-brain receiving an infusion of saline-ascorbate buffer) at two weeks after infusion. Note immunoreactivity for tyrosine hydroxylase (brown). B. Adult rat brain lesioned stereotactically with 6-hydroxydopamine two weeks after infusion. Note loss of immunoreactivity for tyrosine hydroxylase in the central lesioned area, but retention of immunoreactivity peripheral to the lesion.

FIG. 43. Adult rat brain lesioned stereotactically with 6-hydroxydopamine and then injected two weeks later with control buffer solution. Note needle tract (green, arrows) devoid of immunoreactivity for tyrosine hydroxylase and showing the beginnings of a glial scar.

FIG. 44. Adult rat brain lesioned stereotactically with 6-hydroxydopamine and then injected two weeks later with the regenerative pluripotent stem cell, Scl-40β. Note needle tract (green) containing cells that express immunoreactivity for tyrosine hydroxylase (arrows), as well as the presence of cells immunoreactive for tyrosine hydroxylase in adjacent tissue.

FIG. 45. Adult rat hemi-brains from the Parkinson's study were examined for the presence of cells immunoreactive for b-galactosidase outside the area of the substantia nigra to detect movement of the stem cells. The Scl-40b clone of regenerative pluripotent stem cells derived from adult rats was identified throughout its stem cell phase and differentiation phase using an antibody to b-galactosidase. The tissue was harvested and stained with antibody to b-galactosidase (brown cells) and counterstained with methyl green (green/yellow). Immunoreactivity to b-galactosidase was expressed within the cytoplasm of differentiated cells, i.e., glia, pyramidal neurons, and endothelial cells lining newly formed patent capillaries. Cells immunoreactive for Lac-Z were located in areas adjacent to the infusion site in the ipsilateral 6-OHDA/Scl-40b hemi-brains. A. White matter containing glial cells, interneurons, and capillaries with intracellular b-galactosidase-stained material. B. Cortical gray matter containing pyramidal (non-dopaminergic) neurons containing b-galactosidase-stained material. C. Cortical gray matter containing pyramidal (non-dopaminergic) neurons containing b-galactosidase-stained material.

FIG. 46. A, note pluripotent stem cells in vitro in undifferentiated state; B, pluripotent stem cells becoming vasculature after injection into frozen left ventricle; C, pluripotent stem cells incorporating into healing myocardium; and D, pluripotent stem cells becoming incorporated into the healing stromal cardiac skeleton of the heart. Reprinted with permission from Young et al. 2004.

FIG. 47. Systemic tail vein injection of Lac-Z genomically-labeled pluripotent stem cells into the tail vein of an adult rat several weeks after frozen left ventricle. E. Incorporation of differentiated labeled stem cells (green) into the fibrous connective tissue cardiac skeleton of the heart. F. Incorporation of differentiated labeled stem cells (green) into the myocardium of the heart (red).

FIG. 48. Beta-galactosidase histochemistry (blue) of LacZ-genomically-labeled Scl-40b-ELSCs in vitro. Note Blue-green stained nuclei, greater than 90% Of cells retained genomic material within their respective nuclei.

FIG. 49. Beta-galactosidase histochemistry (bluie) of LacZ-genomically-labeled Scl-40b-ELSCs into vasculogenic tissues of the heart two weeks after transient left anterior descending arterial ligation.

FIG. 50. Fluorescence microscopy of incorporation of LacZ genomically-labeled pluripotent epiblast-like stem cells into the left anterior-descending coronary artery after transient ligation. Note red-stained beta-galactosidase appearing cells in the tunica intima, tunica media, and tunica adventitia of the small muscular artery. Note also green wavy lines denoting internal and external elastic membranes.

FIG. 51. Beta-galactosidase histochemistry (blue) of LacZ-genomically-labeled Scl-40b-ELSCs: A—into myocardium and B—into connective tissues of the heart two weeks after transient left anterior descending arterial ligation.

FIG. 52. Beta-galactosidase histochemistry (blue) of LacZ-genomically-labeled Scl-40b-ELSCs into myocardium of left ventricle of the heart at Day 3, Day 14, and Day 28 after transient left anterior descending arterial ligation.

FIG. 53. Decellularized pancreatic matrices. A—SEM of pancreatic matrix-A. Note both thick and thin matrix filaments (mf). B—Bright filed micrograph of pancreatic matrix-A. One can see the structural framework of blood vessels (bv) and matrix filaments (mf). C—SEM of pancreatic matrix-B. Note predominantly thin matrix filaments (mf). D—Brightfield micrograph of pancreatic matrix-B. It is intact and one can see predominantly matrix filaments (mf).

FIG. 54. Multi-layered confluent ELSCs (Scl-40b) and BLSCs (Scl-44b) plated upon decellularized porcine pancreatic matrix.

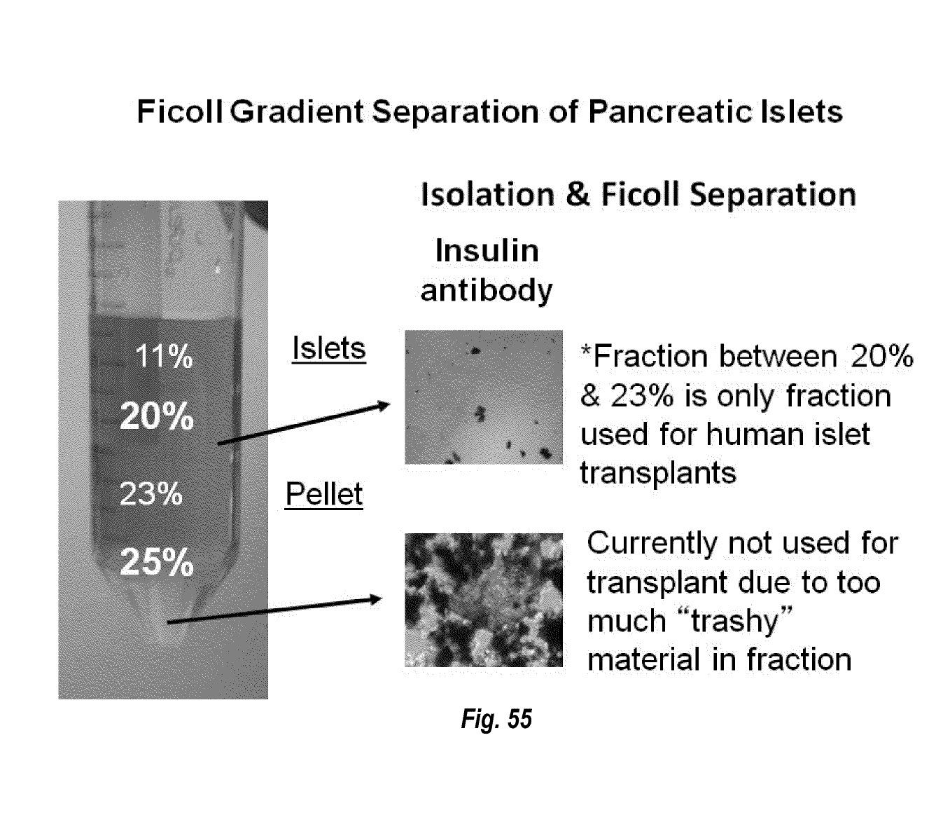

FIG. 55. Ficoll gradient separation of native pancreatic islets and “trashy”-like material. The fraction between 20% and 23% Ficoll (used for human pancreatic islet transplants) was used for the rat islets tested in this material. Note that a majority of the islets are in the pelleted material below the 25% ficoll layer.

FIG. 56. Histogram of radioimmunoassay of released glucose per nanogram DNA after exposure to glucose as the insulin secretagogue. 5 mM glucose at 24 hours and 1 hour are controls for insulin release in a sequential release of insulin from the islets. After the second 5 mM glucose is removed and the cultures washed with sterile phosphate buffered saline at pH 7.4, cultures were incubated with 25 mM glucose as the challenge material. Native control islets secreted 0.3 micrograms of insulin per nanogram of DNA; islet organoids grown on Matrix-A secreted 23.6 micrograms of insulin per nanogram of DNA, while islet organoids grown on Matrix-B secreted 77.3 micrograms of insulin per nanogram of DNA.

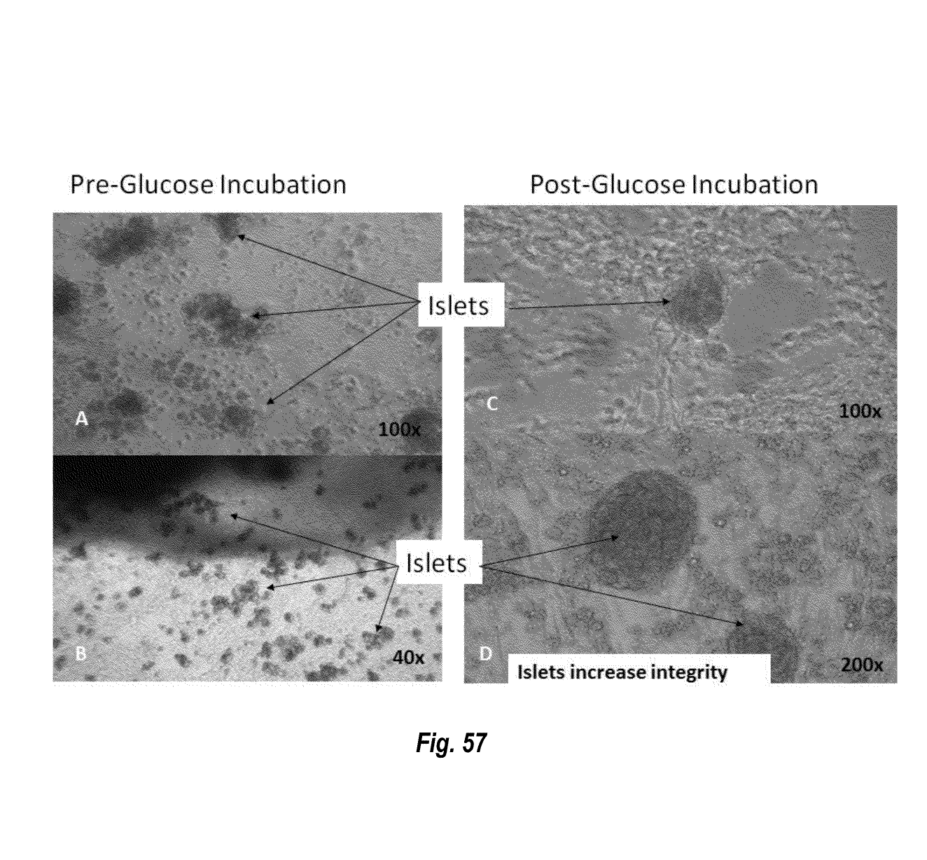

FIG. 57. Isolated rat pancreatic islets were grown on decellularized native porcine pancreatic matrices utilizing the standard BLSC-based culture medium. A & B are rat islets 48 hours after plating. C & D are rat islets 72-96 hours after plating. Note that islets in C & D are increasing their three-dimensional integrity, as if native islet cells were inducing surrounding BLSCs and ELSCs to convert to islet cells. These decellularized pancreatic matrices seeded with BLSCs, ELSCs, and native islets demonstrated an increase in the integrity of the islet structures throughout the 30 days in culture, before termination of the experiment.

FIG. 58. Nomenclature and Definition Slide for Adult Regenerative Pluripotent Stem Cells.

FIG. 59. Identifying BLSCs, Tr-BLSCs on a hemocytometer and counting the types of stem cells present.

FIG. 60. HM001, isolated adult human stem cells from blood after stimulation to multiply in vivo by ingestion of the compound. A. Cells stained 1:1 with 0.4% Trypan blue solution, diluted 1:1000 with sterile saline and mounted onto a hemocytometer, magnification 200×. Note cells that are white “glowing” spheres=ELSCs are Trypan blue negative; cells with dark blue peripheral staining, but clear centers=Tr-BLSCs are both Trypan blue positive and Trypan blue negative; cells that are spherical and solid blue=large-BLSCs and small-BLSCs and are cells that are Trypan blue positive. Original magnification 200×. B. Cells stained with CEA-CAM-1 antibody, diluted 1:1000 with sterile saline and mounted onto a hemocytometer, magnification 200×. Note cells with a peripheral crown-like halo of staining positive for CEA-CAM-1, but center area is clear=CoronaCS cells, which are 3-5 microns in size on flow cytometry of live cells. Also note very small cells that display CEA-CAM-1 throughout=small Blastomeric-LikeSCs, which are 0.2 to 1.0 microns in size on flow cytometry of live cells.

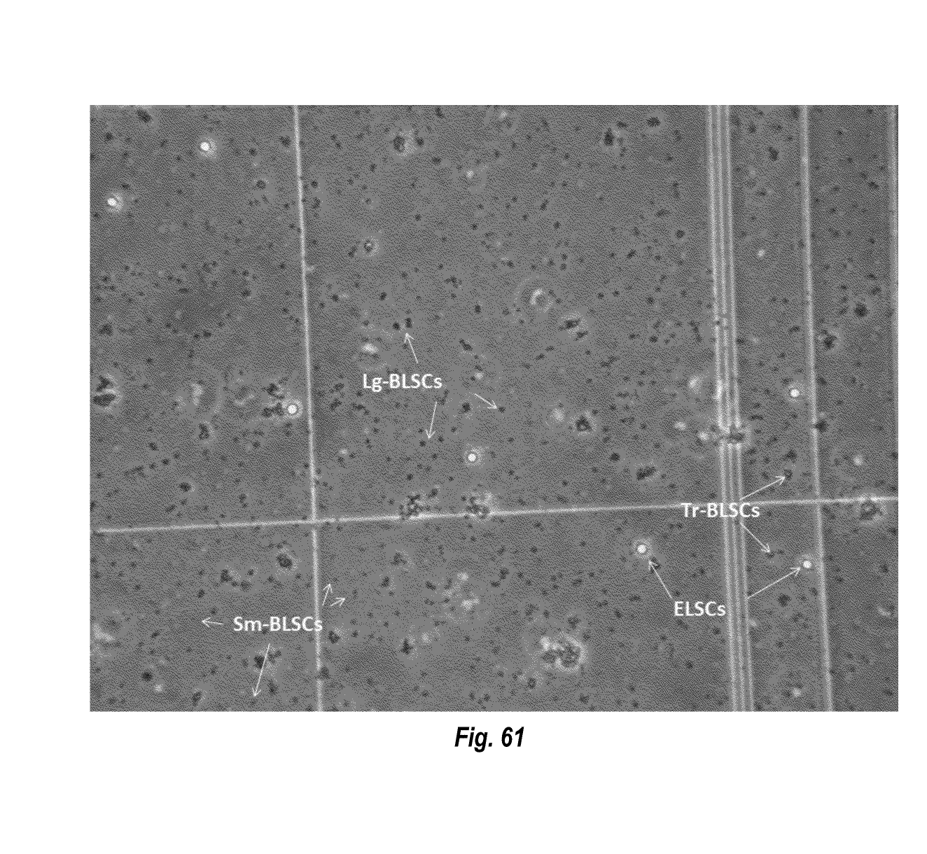

FIG. 61. HM001, adult human stem cells isolated and stained 1:1 with 0.4% Trypan blue solution, diluted 1:1000 with sterile saline and mounted onto a hemocytometer, magnification 200×. Note cells that are white “glowing” spheres=ELSCs; cells with peripheral dark blue staining with white centers=CSCs; cells that are spherical and solid blue=large and small BLSCs.

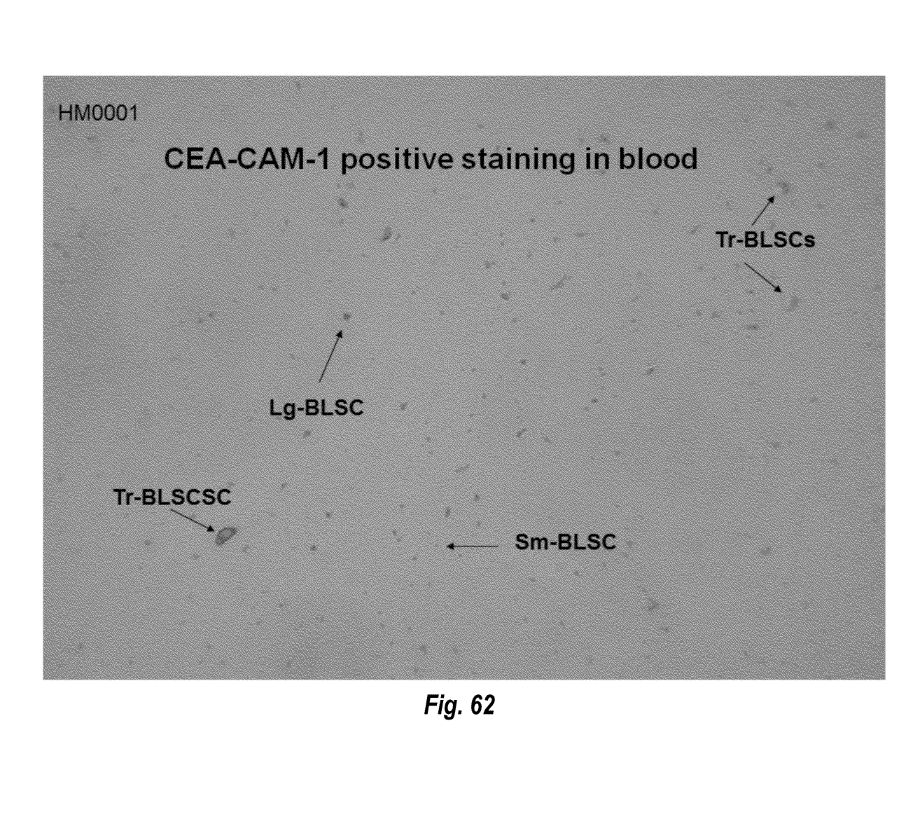

FIG. 62. HM001, adult human stem cells isolated from blood. Cells stained with CEA-CAM-1, diluted to 1:1000. and plated onto hemocytometer. Tr-BLSCs and BLSCs are CEA-CAM-1+.

FIG. 63. Feline and Canine stained plasma fractions.

A: Feline plasma fraction stained with 0.4% Trypan blue. BLSCs are small dark round circles and BLSC-Tr are small glowing structures or structures that have a dark periphery and a clear center, 100× mag. B: Feline plasma fraction stained with CEA-CAM-1. BLSCs are small dark-red round circles and BLSC-Tr are structures with a dark-red periphery and a clear center, 100× mag. C: Canine plasma fraction stained with 0.4% Trypan blue. BLSCs are small dark round circles and BLSC-Tr are structures with a dark periphery and a clear center, 100× mag. D: Canine plasma fraction stained with CEA-CAM-1. BLSCs are small dark-red round circles and BLSC-Tr are structures with a dark-red periphery and a clear center, 100× mag.

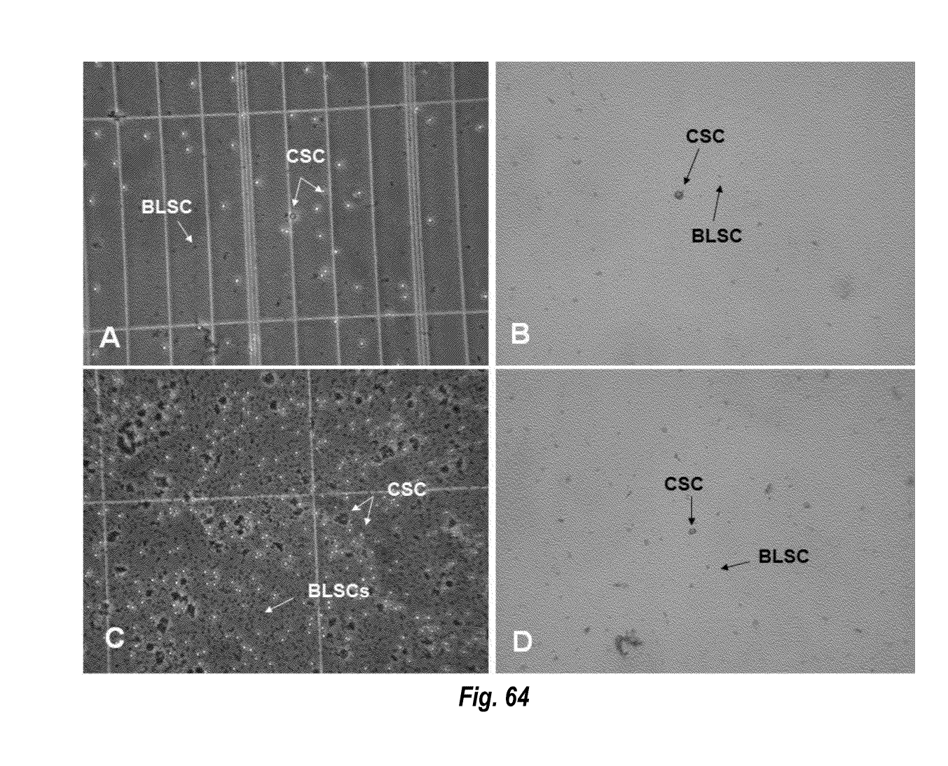

FIG. 64. Ovine (Sheep) and Caprine (Goat) stained plasma fractions.

A: Ovine plasma fraction stained with 0.4% Trypan blue. BLSCs are small dark round circles, BLSC-Tr are either small glowing structures or structures with a dark periphery and a clear center, 100× mag. B: Ovine plasma fraction stained with CEA-CAM-1. BLSCs are small dark-red round circles, BLSC-Tr are structures with a dark-red periphery and a clear center, and ELSCs are unstained, 100× mag. C: Caprine plasma fraction stained with 0.4% Trypan blue. BLSCs are small dark round circles, BLSC-Tr are either small glowing structures or structures with a dark periphery and a clear center, 100× mag. D: Caprine plasma fraction stained with CEA-CAM-1. BLSCs are small dark-red round circles and BLSC-Tr are structures with a dark-red periphery and a clear center, 100× Mag.

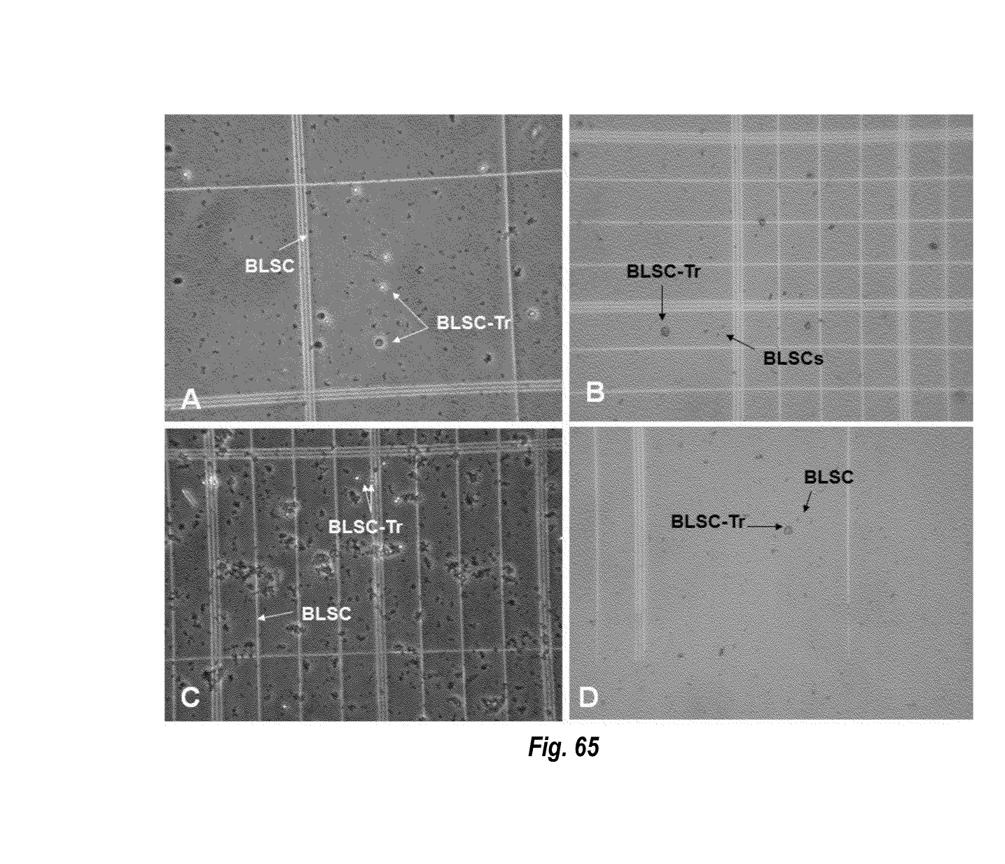

FIG. 65. Bovine and Equine stained plasma fractions. A: Bovine plasma fraction stained with 0.4% Trypan blue. BLSCs are small dark round circles and BLSC-Tr are small glowing structures or structures with a dark periphery and a clear center, 100× mag. B: Bovine plasma fraction stained with CEA-CAM-1. BLSCs are small dark-red round circles and BLSC-Tr are structures with a dark-red periphery and a clear center, 100× mag. C: Equine plasma fraction stained with 0.4% Trypan blue. BLSCs are small dark round circles and BLSC-Tr are small glowing structures or structures with a dark periphery and a clear center, 100× mag. D: Equine plasma fraction stained with CEA-CAM-1. BLSCs are small dark-red round circles and BLSC-Tr are structures with a dark-red periphery and a clear center, 100× mag.

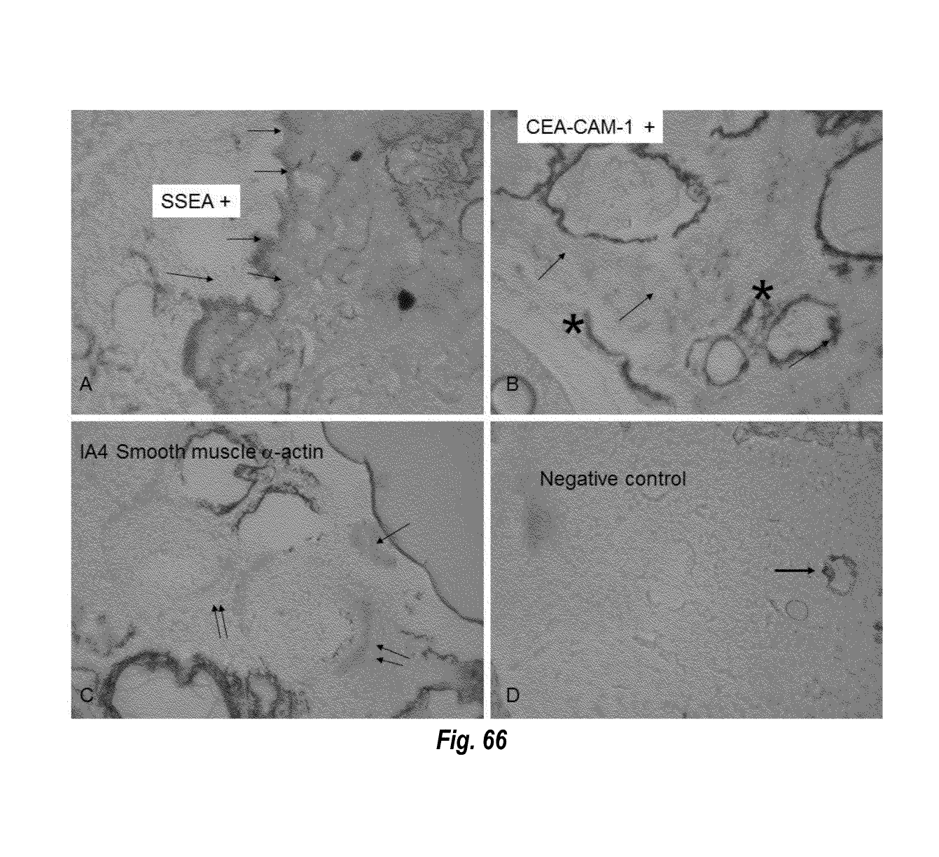

FIG. 66. An adult rat (RM1100) bronchopulmonary segment undergoing repair with resident SSEA positive and CEA-CAM-1 positive cells (BLSCs, Tr-BLSCs and ELSCs). A. SSEA positive cells at border between two bronchopulmonary segments. B. CEA-CAM-1 positive cells within the interstitial tissue between emphysematous lesions (holes) in the lung, i.e., shrinking the size of the holes and increasing the amount of interstitial tissues between lesions. C. IA4 positive smooth muscle alpha-actin in two newly forming blood vessels (double arrows) adjacent to newly forming alveolar duct (single arrow), positive procedural control. D. No primary antibody, negative procedural control. But see that there is more interstitial tissue with forming structures than emphysematous lesions (single arrow).

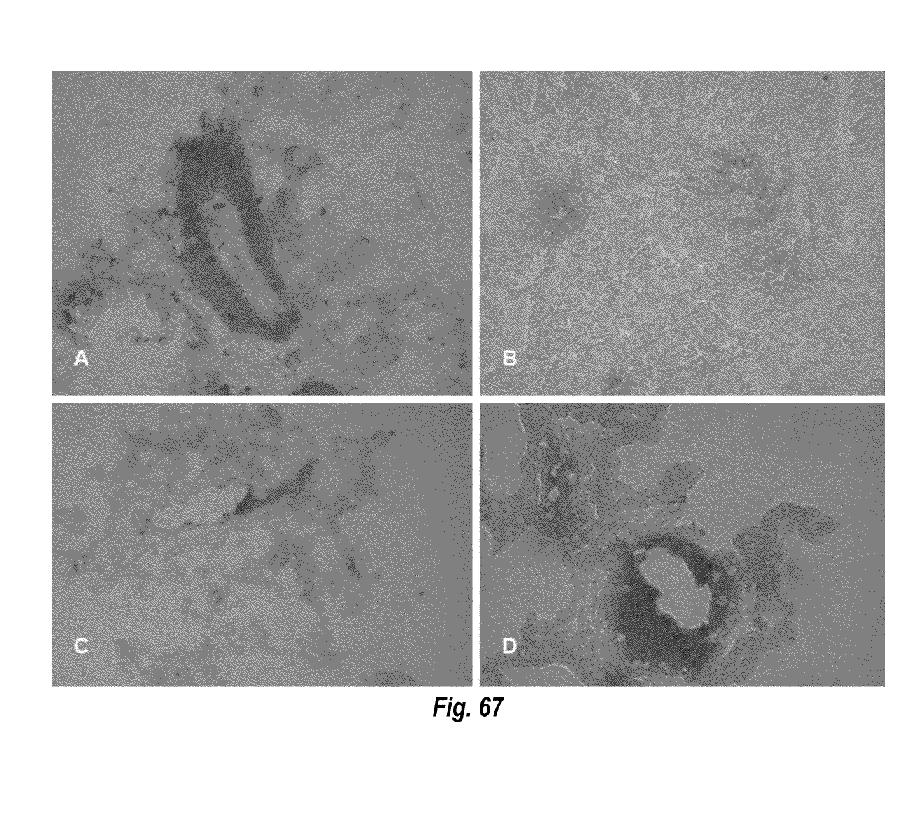

FIG. 67. In regenerating lung tissue, CEA-CAM+ cells (A,B) and SSEA-4+ cells (C,D) are located amongst the tissues. Adult regenerative pluripotent stem cells incorporate into epithelial lining of alveolar sacs, alveolar ducts, respiratory bronchioles, terminal bronchioles, bronchioles, tertiary bronchi, and secondary bronchi. A: Regenerating lung tissue—CEA-CAM+ cells, 100× mag. B: Regenerating alveolar ducts—CEA-CAM+ cells, 200× mag. C: Regenerating bronchiole—SSEA-4+ cells, 40× mag. D: Regenerating alveloar duct—SSEA-4+ cells, 200× mag.

FIG. 68. Transitional-blastomere-like stem cells (red in the figure, gate R9) represent an intermediate population of cells between the BLSCs (gate R8) and the ELSCs (gate R11).

FIG. 69. Adult porcine model of 90 min of tissue trauma, i.e., splenectomy followed by panceatectomy. Five grams of muscle tissue from the rectus abdominis muscle was removed prior to overt trauma and five grams of muscle from the left rectus abdominis muscle was removed following trauma. The tissue was digested with collagenase and dispase, the tissues washed free of the enzymes, the cells stained and counted.

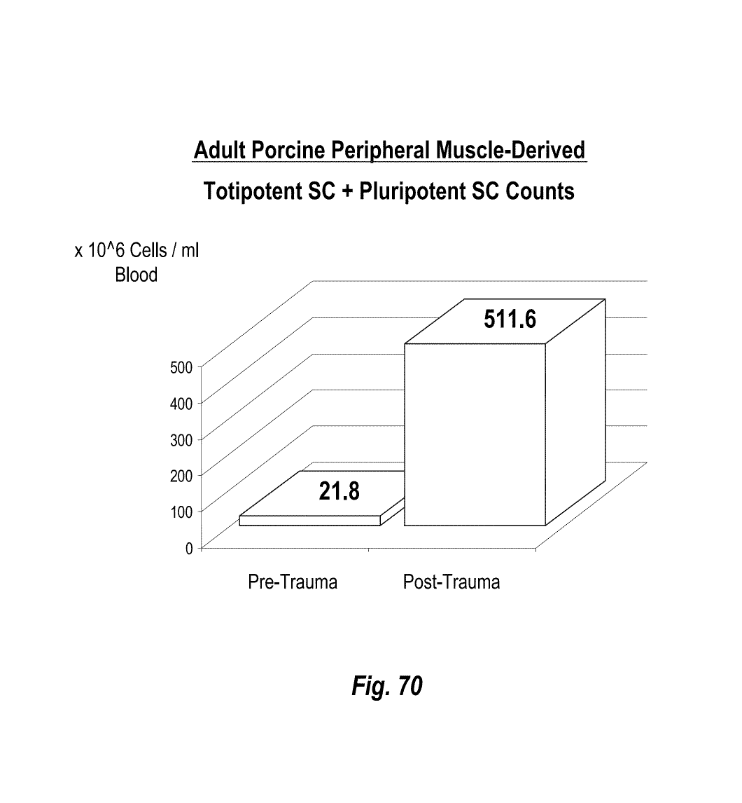

FIG. 70. Adult porcine model of 90 min of tissue trauma, i.e., splenectomy followed by panceatectomy. Ten ml of blood was removed by venipuncture from the left external jugular vein was removed prior to overt trauma and ten ml of blood was removed by venipuncture from the left external jugular vein was removed following overt trauma. The blood was placed into a 4 degree centrifuge refrigerator for 48 hours to separate the stem cells from the hematopoieitc cells, the stem cell fraction was removed, stained and the stem cells counted.

FIG. 71. Absolute counts of Trypan blue-positive BLSCs in single adult human male (HM0010) taking 1 capsule of Stem Enhance. Values are in absolute numbers of cells, rather than percentages of cells at different time periods. When taken recommended dosage of 8 capsules per day the number of stem cells in the blood at 60 min approximates 1500% of normal for 20 individuals (e.g., n=20, HM0011 to HM0021). When taken long term, this number (˜1500%) holds its value for two weeks, then plummets to approximately 1% of normal value and continues at that value as long as the Stem Enhance is taken. During the first two weeks, with high amounts of stem cells circulating in the blood, healing from trauma occurs very quickly. After 14 weeks, healing from trauma was non-existent (anecdotal comments from HM0011 to HM0021). These results suggest that high dose of Stem Enhance for short period of time (e.g., 14 weeks) depleted the supply of mother stem cells as well as the transient daughter stem cells within the tissue connective tissue niches leaving very few mother stem cells behind.

FIG. 72. Ingestion of 1 capsule each of the compound into six horses (EF0006, EF007, EF008, EF009, EG0010, EG0011) using sweet feed. Blood samples were taken at zero minutes (blue), 60 min (burgundy), and 360 min [6-hours] (yellow). Only Trypan blue-positive cells (BLSCs) were counted.

FIG. 73. Serial dilution single cell clonogenic analysis for BLSCs, ELSCs, and Tr-BLSCs. Cell suspension isolated from the skeletal muscle of outbred adult Sprague-Dawley rat. RM0001. Cell suspension was diluted to single cell concentration of one cell per 5 microliters of medium. Single cells in 5 microliters of medium were plated per well of 96-well plates, grown post confluence, replated as single cells per well, grown post confluence, 3-4 times to insure that population of cells came from a single cell.

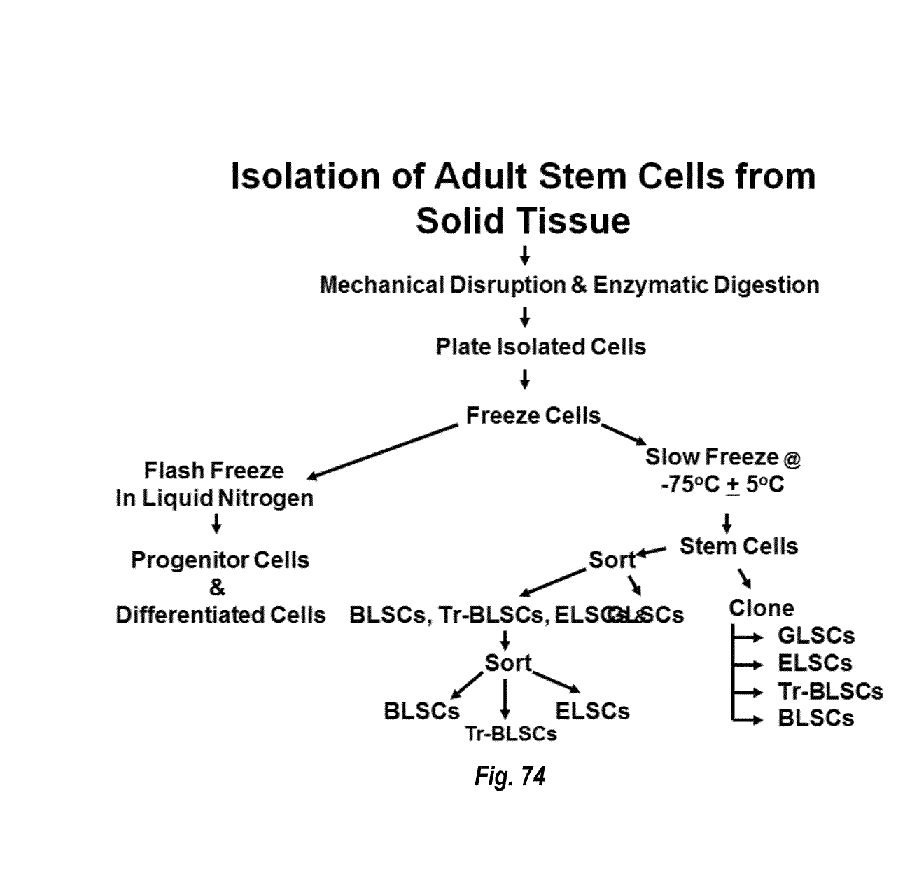

FIG. 74. Isolation protocol for differentiated cells, progenitor cells, germ layer lineage stem cells, epiblast-like stem cells, transitional blastomere-like stem cells, and blastomeric-like stem cells. The flow chart is based on cryopreservation, and cloning by repetitive single cell clonogenic analysis or cell sorting using unique cell surface markers to separate GLSCs, ELSCs, Tr-BLSCs, and BLSCs.

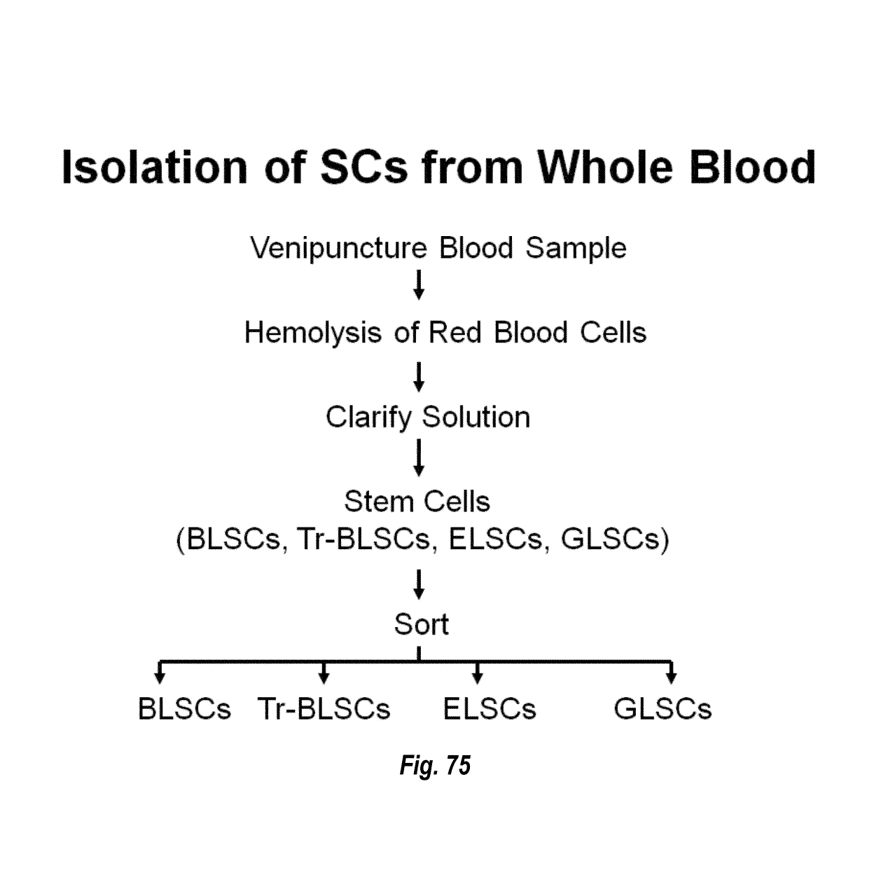

FIG. 75. Isolation of Stem cells, i.e., BLSCs, Tr-BLSCs, ELSCs, and GLSCs, by hemolysis followed by cell sorting with particular cell surface markers.

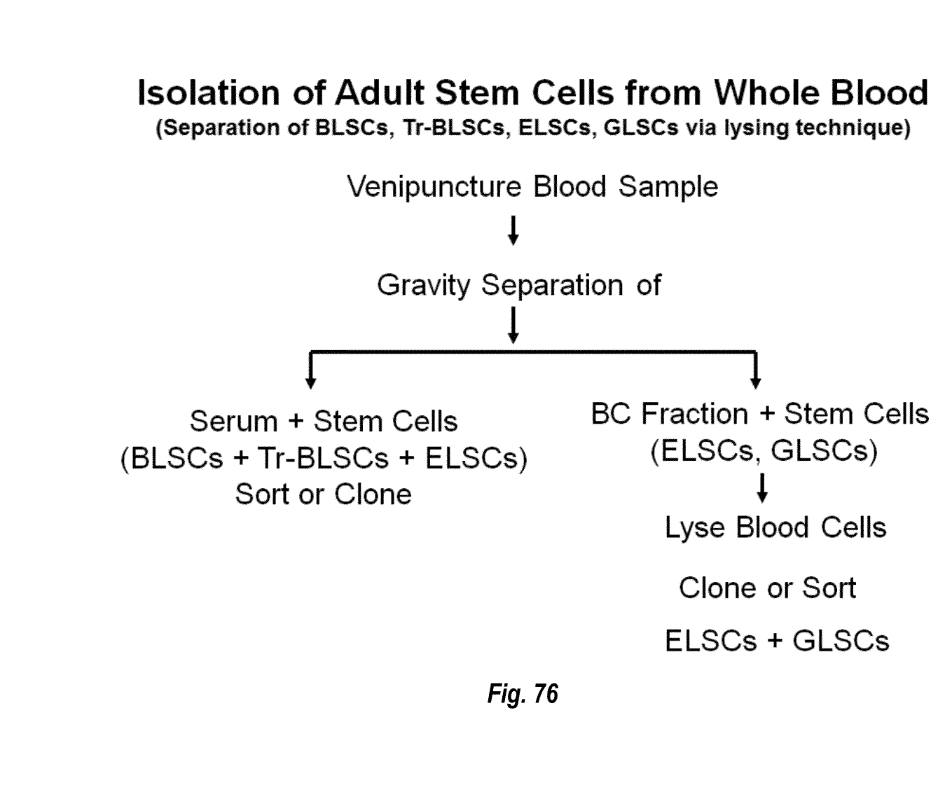

FIG. 76. Alternate method of separation of BLSCs, Tr-BLSCs, ELSCs, and GLSCs from whole blood. First separate whole blood into hematopoietic cellular component and serum by gravity. Then lyse hematopoietic cells and sort both serum and hematopoietic fractions using particular cell surface markers to isolate and separate BLSCs, Tr-BLSCs, ELSCs, and GLSCs.

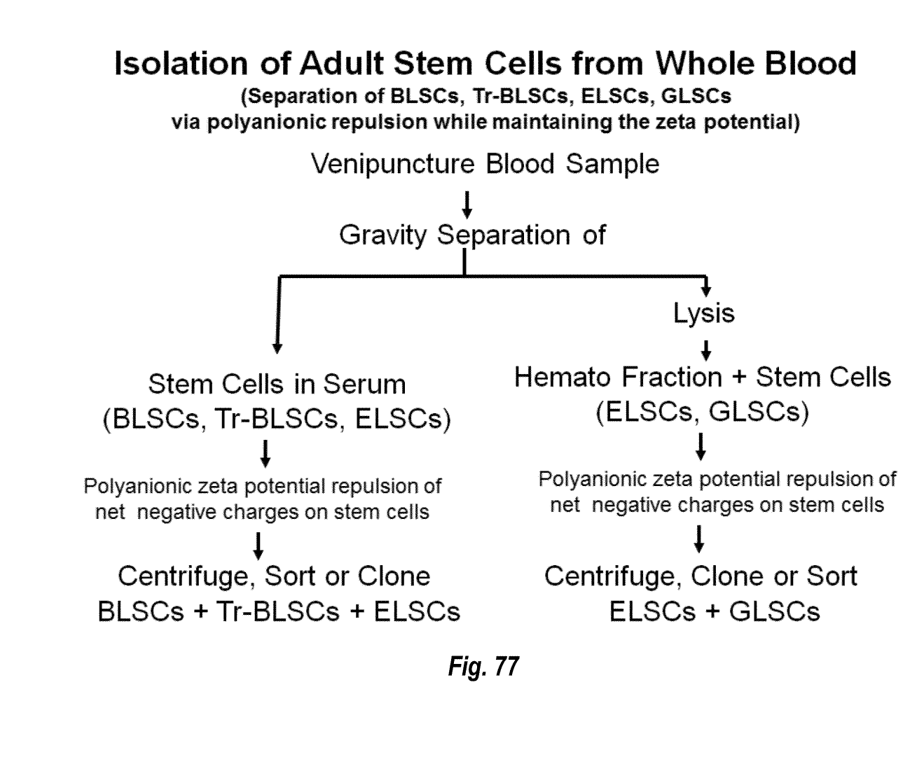

FIG. 77. A second alternate method of separation of BLSCs, Tr-BLSCs, ELSCs, and GLSCs from whole blood. Collect whole blood by venipuncture and place into sterile EDTA tubes, mix contents, and then place into refrigerator for 20 min to 96 hours, dependent on species for gravity separation. Remove serum layer from tube, place into second sterile tube containing EDTA. Place tubes into a 4 degree centigrade refrigerator for 24 hours for multiple polyanionic repulsion of net negative charges on stem cells. After 24 hours remove individual layers of cells from tube. Can also be centrifuged to achieve similar results, or sorted with specific cell surface markers, or cloned from single cells. The hematopoietic fraction can be separated by the same technique, but the hematopoietic cells need to be lysed first before the polyanionic repulsion can begin to take place.

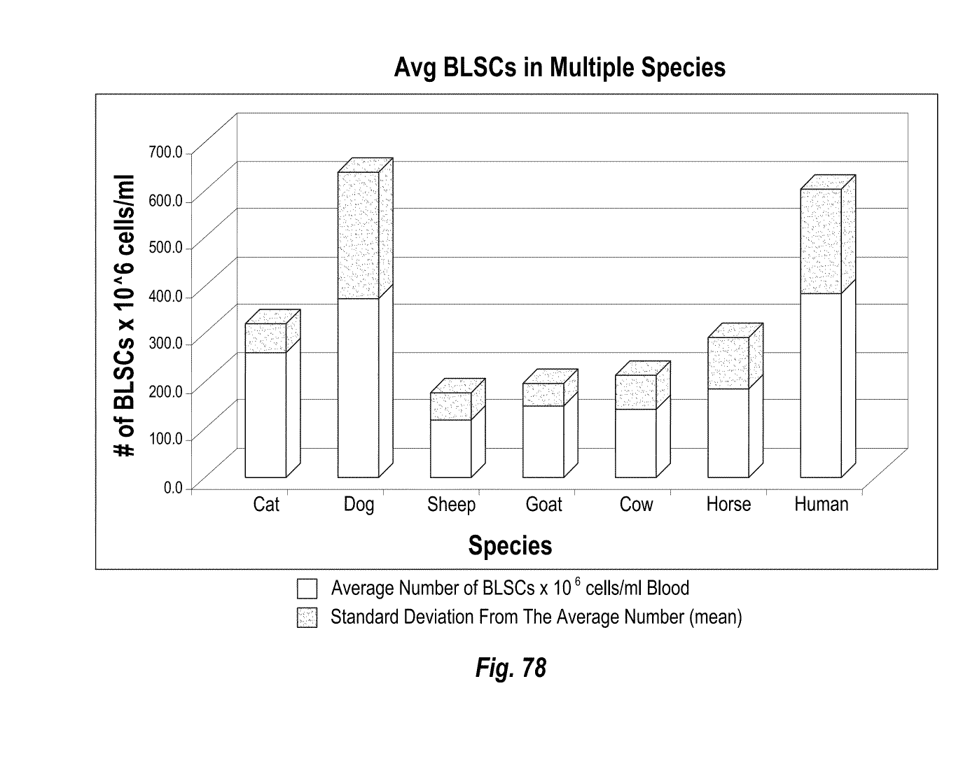

FIG. 78. Blood samples, denoted as (species, gender, sequential code number) were taken from cats/felines (FM0001, FF0002, FM0003, FM0004, FM0005), dogs/canines (CM0001, CF0002, CM0003), sheep/ovines (OF0001, OF0002, OF0003, OF0004), goats/caprines (GM0001, GM0002, GM0003, GM0004), cow/bovine (BM001, BF0002, BM0003, BF0004), horse/Equine Gelding (EG0001, EG0002, EG0003, EF0004, EF0005, EF0006, EF0007, EF0008, EF0009), and human (HM0001, HM0011, HF0012, HM0013, HF0014, HM0015, HF0016, HF0017)

### DETAILED DESCRIPTION

The inventor has unexpectedly discovered that adult-derived regenerative pluripotent transitional blastomere-like stem cells can be obtained from the blood of mammals, particularly from human, (but also mouse, rat, rabbit, cat, dog, sheep, goat, pig, cow, and horse) (FIG. 78), wherein such stem cells have a combination of both blastomeric-like stem cell and epiblast-like stem cell characteristics and wherein the stem cells are isolated from the blood of the mammal or human without killing the mammal or human. Typically, such adult-derived regenerative pluripotent transitional blastomere-like stem cells are isolated from the vasculature of a post-natal (most typically adult, but also infants, adolescents, and pre- and post-puberal teenagers) humans and mammals and are 3-5 microns in size in the unfixed state. It should be particularly appreciated that the adult-derived regenerative pluripotent transitional blastomere-like stem cells according to the inventive subject matter that even though they share cell surface markers with totipotent blastomeric-like stem cells (i.e., CEA-CAM-1, CEA, HCEA, and CD66e), they can NOT give rise to germ line progeny, including spermatogonia.

The term “post-natal” as used herein refers to a stage in development of an organism after birth (which may also include premature birth (i.e., at least 60% of normal gestation)). Most typically post-natal stem cells according to the inventive subject matter are isolated from an adult, but earlier stages (e.g., newborn, infant stages, adolescent, prepubescent, or post puberty) are also deemed suitable. Furthermore, the term “totipotent” as used herein in conjunction with a cell refers to a stem cell that has the ability to give rise to placental and/or germ line cells. In addition, the term “pluripotent” as used herein in conjunction with a cell refers to a stem cell that has the ability to give rise, inclusively, to all somatic cells of the embryo/adult, but NOT the embryonic portion of the placenta or the germ line cells, i.e., germ cells (spermatogonia or ova or any of their differentiated cell types).

Remarkably, the adult-derived regenerative pluripotent transitional blastomere-like stem cells derived from post-natal, rather than embryonic tissues are not committed to any tissue lineage and are of a presumed normal karyotype, since both blastomeric-like stem cells and epiblast-like stem cells are of a normal karyotype. Contemplated cells typically express telomerase, Oct-3/4, Sonic hedge-hog, CEA-CAM-1, and/or the CD66e cell surface markers (i.e., HCEA, CEA) and express stage-specific embryonic antigens SSEA (i.e., SSEA-1, SSEA-3, and/or SSEA-4), and the cell surface marker for neutral endopeptidase, CD10. In contrast, adult-derived regenerative pluripotent transitional blastomere-like stem cells typically fail to express BMI-1, IDE1, IDE3, ABCG2, CXCR-4, BCL-2, Nanog, Nanos, CD1a, CD2, CD3, CD4, CD5, CD7, CDB, CD9, CD11b, CD11c, CD13, CD14, CD15, CD16, CD18, CD19, CD20, CD22, CD23, CD24, CD25, CD31, CD33, CD34, CD36, CD38, CD41, CD42b, CD45, CD49d, CD55, CD56, CD57, CD59, CD61, CD62E, CD65, CD68, CD69, CD71, CD79, CD83, CD90, CD95, CD105, CD106, CD117, CD123, CD135, CD166, Glycophorin-A, MHC-I, HLA-DRII, FMC-7, Annexin-V, and/or LIN cell surface markers.

It should be especially appreciated that the adult-derived regenerative pluripotent transitional blastomere-like stem cells according to the inventive subject matter remain quiescent in serum-free defined medium in the absence of differentiation inhibitory agents (e.g., leukemia inhibitory factor, or anti-differentiation factor), and when implanted into animals do not form cancerous tissues. In concordance, implanted adult-derived regenerative pluripotent transitional blastomere-like stem cells remain quiescent after implantation or incorporate into all tissues undergoing repair.

It should be further noted that the adult-derived regenerative pluripotent transitional blastomere-like stem cells presented herein can also be stimulated in vivo as well as in vitro to proliferate (most typically in response to one or more growth factors (in vitro) or in response to the compound (in vivo)). Remarkably, when stimulated, post-natal adult-derived regenerative pluripotent transitional blastomere-like stem cells exhibit extended self-renewal as long as they remain lineage-uncommitted. Furthermore, the adult-derived regenerative pluripotent transitional blastomere-like stem cells are not contact inhibited at confluence, they require a substratum for growth in vitro and demonstrate telomerase activity.

The inventor further discovered that adult-derived regenerative pluripotent transitional blastomere-like stem cells have the ability to generate all tissues of the conceptus, and all somatic cells of the embryo/fetus from all three germ layer lineages, EXCEPT embryonic/fetal portions of the placenta, and germ cells, In this regard; they mimic pluripotent epiblast-like stem cells.

After extended exposure to a range of dexamethasone concentrations, adult-derived regenerative pluripotent transitional blastomere-like stem cells differentiated into more than 50 discrete cell types. The induced cell types exhibited characteristic morphological and phenotypic expression markers for pluripotent epiblastic-like stem cells, ectodermal germ layer lineage stem cells, epidermal progenitor cells, epidermal cells, neuronal progenitor cells, dopaminergic neurons, pyramidal neurons, other types of neurons, astrocytes, oligodendrocytes, radial glial cells, ganglion cells, endodermal germ layer lineage stem cells, gastrointestinal epithelial cells, hepatic progenitor cells, hepatocytes, bile canalicular cells, oval cells, pancreatic progenitor cells, pancreatic ductal cells, pancreatic alpha-cells, pancreatic beta-cells, pancreatic delta-cells, three-dimensional pancreatic islets, mesodermal germ layer lineage stem cells, muscle progenitor cells, skeletal muscle, smooth muscle, cardiac muscle, adipogenic progenitor cells, white fat, brown fat, chondrogenic progenitor cells, hyaline cartilage, articular cartilage, growth plate cartilage, elastic cartilage, fibrocartilage, fibrogenic progenitor cells, tendon, ligament, scar tissue, dermis, osteogenic progenitor cells, cancellous bone, trabecular bone, woven bone, lamellar bone, osteoblasts, osteocytes, osteoclasts, endotheliogenic progenitor cells, endothelial cells, hematopoietic progenitor cells, erythrocytes, macrophages, B-cell lymphocytes, and T-cell lymphocytes.

Such induced unidirectional lineage-commitment process necessitates the use of either general induction agents or inductive agents that cause the cell to differentiate into specific tissue lineages. It is contemplated that once adult-derived regenerative pluripotent transitional blastomere-like stem cells are induced to commit to pluripotent epiblast-like stem cells, they have four options. The stem cells can (a) apoptose, (b) remain quiescent, (c) proliferate, or (d) differentiate into ectodermal, endodermal, and/or mesodermal germ layer lineage stem cells. Similarly, once pluripotent epiblastic-like stem cells are induced to commit to form ectodermal, endodermal, and/or mesodermal germ layer lineage stem cells, they have four options as well. The stem cells can (a) apoptose, (b) remain quiescent, (c) proliferate, or (d) differentiate into lineage-committed progenitor cells characteristic of specific tissue lineages. Once committed to specific tissue lineages, they assume the characteristics of lineage-specific progenitor cells, which again can (a) apoptose, (b) remain quiescent, (c) proliferate, or (d) uni-directionally progress down their differentiation pathway, under the influence of specific agents. As committed progenitor cells, their ability to replicate is limited to approximately 50-70 cell doublings (human) or 8-10 cell doublings (rodent) before programmed cell senescence and cell death occurs.

Consequently, it should be recognized that human adult-derived regenerative pluripotent transitional blastomere-like stem cells can be obtained in a relatively simple manner by extraction from autologous circulating blood after stimulation by the compound, and thereafter expanded without ever having to leave the individual. Indeed, previous experiments by the inventor have shown that the cells according to the inventive subject matter can undergo at least 100 population doublings in situ and maintain their undifferentiated state. Therefore, it should be recognized that these cells do not spontaneously differentiate in situ, but remain in an undifferentiated state. Once sufficient quantities of adult-derived regenerative pluripotent transitional blastomere-like stem cells are obtained (with or without expansion), they may be implanted into a human without teratoma formation, and will remain quiescent unless in the presence of damaged, necrotic, and/or inflamed tissue undergoing repair. Alternatively, contemplated adult-derived regenerative pluripotent transitional blastomere-like stem cells may be expanded in vivo and then subjected to differentiation steps to thereby generate pluripotent stem cells (e.g., epiblast-like stem cells), germ layer lineage stem cells (e.g., those forming ectodermal cells, mesodermal cells, and endodermal cells), and/or progenitor cells (e.g., multipotent cells, tripotent cells, bipotent cells, and unipotent cells) in quantities that would otherwise be difficult, if not even impossible to obtain. Moreover, it should be recognized that such cells will be available for implantation into a donor with either an autologous or allogeneic match.

TABLE 1
Characteristics of Differentiated Cells, Progenitor Cells and Stem Cells

| | | | | | | | |
|---|---|---|---|---|---|---|---|
| Charac. | | DC¹ | PC² | GLSC³ | ELSC⁴ | Tr-BLSC⁵ | BLSC⁶ |
| Size: | | | | | | | |
| Rat (R) | | Var⁷ | Var | 8-10 μm | 6-8 μm | 3-5 μm | 0.2-2.0 μm |
| Human (H) | | Var | Var | 8-10 μm | 6-8 μm | 3-5 μm | 0.2-2.0 μm |
| Viability P- | R | 6-12 hr | 24 hr | 3 days | 7 days | 30 days | 30+ days |
| Mortem⁸ | H | 6-12 hr | 24 hr | 3 days | 7 days | 30 days | 30+ days |
| Presence | | | | | | | |
| in STs⁹ | | Yes | Yes | Yes | Yes | Yes | Yes |
| in CTs¹⁰ | | Yes | Yes | Yes | Yes | Yes | Yes |
| in BM¹¹ | | Yes | Yes | Yes | Yes | Yes | Yes |
| in Bld¹² | | Yes | Yes | Yes | Yes | Yes | Yes |
| Serum-Free | | Qui¹³ | Qui | Qui | Qui | Qui | Qui |
| Medium | | | | | | | |
| Commitment | | NA¹⁴ | DC | PC | GLSC | ELSC | Tr-BLSC |
| Tel'ase¹⁵ | | Absent | Absent | Present | Present | Present | Present |
| Trypan | R | negative | negative | negative | negative | pos/neg | positive |
| Blue | H | negative | negative | negative | negative | pos/neg | positive |
| PD¹⁶ | R | 8-10 | 8-10 | Extensive | Extensive | Extensive | Extensive |
| | H | 50-70 | 50-70 | Extensive | Extensive | Extensive | Extensive |
| PR¹⁷ | R | D-W¹⁸ | D-W | 18-24 hr | 12-14 hr | 12-14 hr | 12-14 hr |
| | H | D-W | D-W | 18-24 hr | 12-14 hr | 12-14 hr | 12-14 hr |
| CD¹⁹ | R | NYD²⁰ | NYD | >400 | >300 | >100 | >300 |
| | H | 50 | NYD | >690 | >400 | >100 | >300 |
| CD after | NA | | 8-10 | 8-10 | 8-10 | 8-10 | 8-10 |
| Commit R | H | NA | 50-70 | 50-70 | 50-70 | 50-70 | 50-70 |
| to Cell | | | | | | | |
| Lineage | | | | | | | |
| CI-C²¹ | R | Yes | Yes | Yes | No | No | No |
| | H | Yes | Yes | Yes | No | No | No |
| GT-1C²² | R | Yes | Yes | Yes | Yes | Yes | Yes |
| | H | Yes | Yes | Yes | Yes | Yes | Yes |
| G-S²³ | R | No | No | No | No | No | 0.2-1.0 μm Yes |
| | R | | | | | | 1.0-2.0 μm No |
| G-S | H | No | No | No | No | No | 0.2-1.0 μm Yes |
| | R | | | | | | 1.0-2.0 μm No |
| R-Prolif²⁴ | R | Yes | Yes | Yes | Yes | Yes | Yes |
| | H | Yes | Yes | Yes | Yes | Yes | Yes |
| R-Prog²⁵ | R | NA | Yes | No | No | No | No |
| | H | NA | Yes | No | No | No | No |
| R-Induc²⁶ | R | NA | No | Yes | Yes | Yes | Yes |
| | H | NA | No | Yes | Yes | Yes | Yes |
| R-Inhib²⁷ | R | NA | Yes | Yes | Yes | Yes | Yes |
| | H | NA | Yes | Yes | Yes | Yes | Yes |
| #Cs-Fr²⁸ | | 10\hat{ }6 | 10\hat{ }6-7 | 10\hat{ }6-7 | 10\hat{ }6-7 | 10\hat{ }6-7 | 10\hat{ }9-10 |
| Freeze | | Liquid | Liquid | Ultra-Pure | Ultra-Pure | Ultra-Pure | Ultra-Pure |
| Agent:²⁹ | | Nitrogen | Nitrogen | DMSO³⁰ | DMSO | DMSO | DMSO |
| Temp³¹ | R | −196 C. | −196 C. | −70 C. | −80 C. | −80 C. | −80 C. |
| | H | −196 C. | −196 C. | −70 C. | −80 C. | −80 C. | −80 C. |
| FrProc³² | R | Flash | Flash | Slow | Slow | Slow | Slow |
| | H | Flash | Flash | Slow | Slow | Slow | Slow |
| ThwProc³³ | R | Flash 37 C. | Flash 37 C. | Flash 37 C. | Flash 37 C. | Flash 37 C. | Flash 37 C. |
| | H | Flash 37 C. | Flash 37 C. | Flash 37 C. | Flash 37 C. | Flash 37 C. | Flash 37 C. |
| Recov³⁴ | R | >90% | >95% | >98% | >98% | >98% | >98% |
| | H | >95% | >95% | >98% | >98% | >98% | >98% |
| RC-1C³⁵ | R | NYD | NYD | A₂A₂ | Scl-40β | NYD | Scl-44β, |
| | H | NYD | NYD | NYD | NYD | NYD | NYD |
| CsFmd³⁶ | R | NA | DC | PC | GLSC | ELSC | Tr-BLSC |
| | H | NA | DC | PC | GLSC | ELSC | Tr-BLSC |
| Genes | | NYD | NYD | Telom | Telom | Telom | Telom |
| Expressed | | | | | Oct-4 | Oct-4 | Bcl-2 |
| | | | | | Sonic-hh | Sonic-hh | Nanog |
| | | | | | | | Nanos |
| | | | | | | | CXCR-4 |
| CD-Ms³⁷ | H | Cell Spec | Cell Spec | CD13+, | CD10+, | CD10+, | CD66e+, |
| | | | | CD56+, | CD1a−, | CD66e+, | CD1a−, |
| | | | | CD90+, | CD2−, | CD1a−, | CD2− |
| | | | | MCH Class-1+, | CD3−, | CD2− | CD3−, |
| | | | | CD1a−, | CD4−, | CD3−, | CD4−, |
| | | | | CD2−, | CD5−, | CD4−, | CD5−, |
| | | | | CD3−, | CD7−, | CD5−, | CD7−, |
| | | | | CD4−, | CD8−, | CD7−, | CD8−, |
| | | | | CD5−, | CD9−, | CD8−, | CD9−, |
| | | | | CD7−, | CD11b−, | CD9−, | CD10−, |
| | | | | CD8−, | CD11c−, | CD11b−, | CD11b−, |
| | | | | CD9−, | CD13−, | CD11c−, | CD11c−, |
| | | | | CD10−, | CD14−, | CD13−, | CD13−, |
| | | | | CD11b−, | CD15−, | CD14−, | CD14−, |
| | | | | CD11c−, | CD16−, | CD15−, | CD15−, |
| | | | | CD14−, | CD18−, | CD16−, | CD16−, |
| | | | | CD15−, | CD19−, | CD18−, | CD18−, |
| | | | | CD16−, | CD20−, | CD19−, | CD19−, |
| | | | | CD18−, | CD22−, | CD20−, | CD20−, |
| | | | | CD19−, | CD23−, | CD22−, | CD22−, |
| | | | | CD20−, | CD24−, | CD23−, | CD23−, |
| | | | | CD20−, | CD25−, | CD24−, | CD24−, |
| | | | | CD23−, | CD31−, | CD25−, | CD25−, |
| | | | | CD24−, | CD33−, | CD31−, | CD31−, |
| | | | | CD25−, | CD34−, | CD33−, | CD33−, |
| | | | | CD31−, | CD36−, | CD34−, | CD34−, |
| | | | | CD33−, | CD38−, | CD36−, | CD36−, |
| | | | | CD34+/−, | CD41−, | CD38−, | CD38−, |
| | | | | CD36−, | CD42b−, | CD41−, | CD41−, |
| | | | | CD38−, | CD45−, | CD42b−, | CD42b−, |
| | | | | CD41−, | CD49d−, | CD45−, | CD45−, |
| | | | | CD42b−, | CD55−, | CD49d−, | CD49d−, |
| | | | | CD45−, | CD56−, | CD55−, | CD55−, |
| | | | | CD49d−, | CD57−, | CD56− | CD56−, |
| | | | | CD55− | CD59−, | CD57−, | CD57−, |
| | | | | CD57−, | CD61−, | CD59−, | CD59−, |
| | | | | CD59−, | CD62e−, | CD61−, | CD61−, |
| | | | | CD61−, | CD65−, | CD62e−, | CD62e−, |
| | | | | CD62e−, | CD66−, | CD65−, | CD65−, |
| | | | | CD65−, | CD68−, | CD68−, | CD68−, |
| | | | | CD66−, | CD71−, | CD71−, | CD71−, |
| | | | | CD68−, | CD79−, | CD79−, | CD79−, |
| | | | | CD71−, | CD83−, | CD83−, | CD83−, |
| | | | | CD79−, | CD90− | CD90−, | CD90−, |
| | | | | CD83−, | CD95−, | CD95−, | CD95−, |
| | | | | CD95−, | CD105−, | CD105−, | CD105−, |
| | | | | CD105−, | CD117−, | CD117−, | CD117−, |
| | | | | CD117−, | CD123−, | CD123−, | CD123−, |
| | | | | CD123−, | CD135−, | CD135−, | CD135−, |
| | | | | CD135−, | CD166−, | CD166−, | CD166−, |
| | | | | CD166−, | GlycophorinA− | GlycophorinA−, | GlycophorinA− |
| | | | | GlycophorinA−, | MHC-I−, | MHC-I−, | MHC-I−, |
| | | | | HLA-DR-II−, | HLA-DR-II−, | HLA-DR-II−, | HLA-DR-II−, |
| | | | | FMC-7−, | FMC-7−, | FMC-7−, | FMC-7−, |
| | | | | Annexin-V−, | Annexin-V, | Annexin-V, | Annexin-V, |
| | | | | Lin− | Lin− | Lin− | Lin− |

Table 1 Legend
¹DC, differentiated cells
²PC, progenitor cells
³GLSC, germ layer lineage stem cell
⁴ELSC, epiblast-like stem cell
⁵Tr-BLSC, adult-derived regenerative pluripotent transitional blastomere-like stem cell
⁶BLSC, blastomeric-like stem cell
⁷Var, variable
⁸Viabil P. Mortem, viability of tissue post mortem (after removal from the body) stored at 4° C.
⁹STs, solid tissues
¹⁰CTs, connective tissues
¹¹BM, bone marrow
¹²Bld, blood
¹³Qui, quiescence
¹⁴NA, not applicable
¹⁵Tel'ase, telomerase
¹⁶PD, population doublings
¹⁷PR, proliferation rate
¹⁸D-W, days to weeks
¹⁹CD, cell doublings
²⁰NYD, not yet determined
²¹CI-C, cell inhibition at confluence
²²GT-1C, growth on type-1 collagen substrate
²³G-S, growth in cell suspension cultures
²⁴R-Prolif, response to proliferation factors
²⁵R-Prog, response to progression agents
²⁶R-Induc, response to induction factors
²⁷R-Inhib, response to inhibitory factors
²⁸#Cs-Fr, number of cells frozen per aliquot
²⁹Freeze Agent, cryopreservation agent
³⁰DMSO, dimethyl sulfoxide, 99.99% pure
³¹Temp, optimum freezing temperature in centigrade
³²FrProc, freezing process
³³ThwProc, thawing process
³⁴Recov, % recovery from freezing process
³⁵RC-1C, rat clones derived from one cell
³⁶CsFmd, cells formed
³⁷CD-Ms, cluster of differentiation (CD) markers

1.-69. (canceled)

70. An isolated Transitional Blastomere-Like Stem Cell (“tr-BLSC”), wherein the tr-BLSC has a size in a range of about 3-5 μm and at least one surface or phenotypic marker selected from the group consisting of CD10+, SSEA-1+, SSEA-3+, and SSEA-4+, a halo of staining of CD66e⁺, CEA-CAM-1⁺, and trypan blue staining.

71. The isolated tr-BLSC of claim 70, wherein the isolated tr-BLSC is capable of proliferating in serum-free medium and does not spontaneously differentiate in serum-free medium in the absence of differentiation inhibitors.

72. The isolated tr-BLSC of claim 70, wherein the isolated tr-BLSC is adult-derived.

73. The isolated tr-BLSC of claim 70, wherein the isolated tr-BLSC is a pluripotent cell and is capable of proliferating in serum-free medium for at least 100 population doublings while maintaining pluripotency.

74. The isolated tr-BLSC of claim 70, wherein the isolated tr-BLSC is derived from a mammal.

75. The isolated tr-BLSC of claim 70, wherein the isolated tr-BLSC is derived from peripheral human blood.

76. The isolated tr-BLSC of claim 70, wherein the isolated tr-BLSC is further characterized by expression of telomerase, Oct-3/4, Sonic hedgehog, CD66e/CD10 joined cell surface markers and lack of expression of BMI-1, IDE1, IDE3, ABCG2, CXCR-4, BCL-2, CD1 a, CD2, CD3, CD4, CD5, CD7, CDB, CD9, CD11b, CD11c, CD13, CD14, CD15, CD16, CD18, CD19, CD20, CD22, CD23, CD24, CD25, CD31, CD33, CD34, CD36, CD38, CD41, CD42b, CD45, CD49d, CD55, CD56, CD57, CD59, CD61, CD62E, CD65, CD68, CD69, CD71, CD79, CD83, CD90, CD95, CD105, CD106, CD117, CD123, CD135, CD166, Glycophorin-A, MHC-I, HLA-DRII, FMC-7, Annexin-V, and/or LIN.

77. A method for inducing the proliferation of Transitional Blastomere-Like Stem Cells (“tr-BLSCs”) in vivo, the method comprising:
administering to a subject a pharmaceutically effective amount of a composition that is capable of inducing proliferation of tr-BLSCs in vivo, wherein the pharmaceutically effective amount of the composition is capable of increasing a number of tr-BLSCs in the peripheral blood of the subject by at least 200% within about 6 hours of administration; and
inducing transfer of the proliferated tr-BLSCs to a peripheral blood of the subject, wherein the proliferated tr-BLSCs have a size in a range of about 3-5 μm and at least one surface or phenotypic marker selected from the group consisting of CD10+, SSEA-1+, SSEA-3+, and SSEA-4+, a halo of staining of CD66e⁺, CEA-CAM-1⁺, and trypan blue staining.

78. The method claim 77, wherein a dosage of the composition comprises a caloric content of 500-2500 kCal, a protein content of 50-100%, 0-50% fat, 1-20% minerals, 1-10% lipids, 1-10% pigments, 1-10% moisture, 50-100% chlorophyll, 10-50 mg alpha-linolenic acid (Omega-3), 1-30 mg gamma-linolenic acid, 400-5000 IU provitamin-A beta carotene, 1-100 mcg thiamine (B1), 1-100 mcg riboflavin (B2), 1-100 mcg niacin (B3), 1-100 mcg pantothenic acid (B5), 1-100 mcg pyridoxine (B6), 1-100 mcg inositol, 1-100 mcg vitamin D, 1-100 IU vitamin E, 1-100 mcg ascorbic acid (vitamin C), 1-1000 mcg biotin, 1-1000 mcg folic acid, 1-1000 mcg choline, 1-1000 mcg cobalamin (B12), 1-1000 mcg vitamin K, 1-1000 mcg boron, 1-1000 mcg calcium, 1-1000 mcg chloride, 1-1000 mcg chromium, 1-1000 mcg cobalt, 1-1000 mcg copper, 1-1000 mcg fluoride, 1-1000 mcg germanium, 1-1000 mcg iodine, 1-1000 mcg iron, 1-1000 mcg magnesium, 1-1000 mcg molybdenum, 1-1000 mcg nickel, 1-1000 mcg potassium, 1-1000 mcg phosphorous, 1-1000 mcg selenium, 1-1000 mcg silicon, 1-1000 mcg sodium, 1-1000 mcg tin, 1-1000 mcg titanium, 1-1000 mcg vanadium, and 1-1000 mcg zinc.

79. The method claim 77, further comprising:
administering one dosage of the composition per day to the subject for a first period of time;
administering two dosages of the composition per day to the subject for a second period of time; and
administering three dosages of the composition per day to the subject for a third period of time.

80. The method of claim 79, wherein the first, second, and third periods of time range from one week to one month.

81. The method of claim 79, wherein the dosage comprises a 500 mg capsule.

82. The method of claim 77, further comprising isolating tr-BLSCs from the subject, the isolating comprising:
withdrawing 1-500 ml of blood from a subject into a sterile container that includes an anticoagulant;
separating the blood into a plasma fraction and a red blood cell fraction;
collecting the plasma from the plasma fraction;
diluting the plasma by adding an equal amount of sterile isotonic saline to the collected plasma; and
centrifuging the diluted plasma at least 1000×g for a period of time sufficient to pellet at least 60% of the tr-BLSCs from the diluted plasma.

83. The method of claim 82, further comprising:
removing a supernatant fraction from the pellet of tr-BLSCs; and
resuspending the pellet of tr-BLSCs in sterile isotonic saline.

84. The method of claim 83, further comprising:
adding DMSO to the resuspended tr-BLSCs;
freezing the tr-BLSCs; and
storing the frozen tr-BLSCs at about −80° C.

85. The method of claim 83, further comprising lyophilizing the resuspended tr-BLSCs.

86. The method of claim 77, wherein the separating includes storing the blood at about 4° C. for a period of time ranging from 20 minutes to 96 hours to separate the blood into the plasma fraction and the red blood cell fraction.

87. A method of treating a disorder selected from the group consisting of chronic obstructive pulmonary disease (COPD), interstitial pulmonary fibrosis (IPF), Parkinson's disease, multiple sclerosis, Alzheimer's disease, dementia, stroke, spinal cord injuries, neuropathies, neuroparesthesias, sciatica, type-I diabetes, myocardial infarction, cardiovascular diseases, autoimmune disorders, bone fractures, cartilage repair, muscle tears, limb restoration, and burn injuries, the method comprising:
providing cells isolated according to the method of claim 20; and
implanting the cells into a subject.

88. The method of claim 87, wherein the cells are implanted into a damaged tissue or tissue undergoing repair.

89. The method of claim 87, wherein the cells are implanted by at least one of nebulization, intravenous infusion, intranasal inhalation, intra-nasal infusion, intra-spinal injection, intra-lumbar cistern injection, intra-thecal injection, intra-articular injection, intramuscular injection, intravascular injection, topical cream or solution, or eye drops.
